(19) United States
(12) Patent Application Publication
(10) Pub. No.: US 2014/0107062 A1
(43) Pub. Date: Apr. 17, 2014

(54) COMPOSITIONS AND METHODS FOR TREATING OR PREVENTING DISEASES OF BODY PASSAGEWAYS

(71) Applicant: Aravasc Inc., Sunnyvale, CA (US)

(72) Inventor: Narmada Shenoy, Sunnyvale, CA (US)

(73) Assignee: ARAVASC INC., Sunnyvale, CA (US)

(21) Appl. No.: 14/133,405

(22) Filed: Dec. 18, 2013

(30) Foreign Application Priority Data
Apr. 27, 2007 (US) PCT/US07/10574

(51) Int. Cl. A61K 9/70; A61K 31/454; A61L 27/54; A61K 31/513; A61K 31/7072; A61K 47/34; A61K 31/337

(52) U.S. Cl. CPC A61K 9/7007; A61K 47/34; A61K 31/454; A61K 31/337; A61K 31/513; A61K 31/7072; A61L 27/54
USPC 514/50; 514/772.3; 514/323; 514/449; 514/274; 623/1.42

(57) ABSTRACT

The present invention provides compositions and methods for treating or preventing diseases associated with vascular and non-vascular body passageways, the method comprising the step of delivering to a body passageway a therapeutic agent delivered locally through a polymer matrix from an implanted stent or other structure.

This application is a Continuation-in-part and claims the benefit of co-pending U.S. Non-provisional patent application Ser. No. 12/226,752, filed Sep. 30, 2009, which itself was a 35 U.S.C. §371 national stage filing of International Patent Application Serial No. PCT/US2007/010574, filed Apr. 27, 2007, entitled “Compositions and Methods for Treating or Preventing Diseases of Body Passageways”, which claimed priority to U.S. Provisional Patent Application Ser. No. 60/745,834, filed Apr. 27, 2006, entitled “Composition and Method for Treating or Preventing Diseases of Body Passageways by Loco-Regional Drug Delivery” which are all herein incorporated by reference in their entirety.

### TECHNICAL FIELD

The present invention relates generally to compositions and methods for treating or preventing diseases of vascular or non-vascular body passageways, and more specifically, to compositions comprising therapeutic agents which may be delivered locally through a polymer matrix to the body passageways via a medical device implant that is bio-degradable, partly bio-degradable or permanent comprised of a polymer or a metal.

### BACKGROUND ART

There are many passageways within the body which allow the flow of essential materials. These include, for example, arteries and veins, the esophagus, stomach, small and large intestine, biliary tract, ureter, bladder, urethra, nasal passageways, trachea and other airways, and the male and female reproductive tract. Injury, various surgical procedures, or disease can result in the compression, narrowing, weakening and/or obstruction of such body passageways, resulting in serious complications and/or even death.

For example, many types of tumors (both benign and malignant) can result in damage to the wall of a body passageway or obstruction of the lumen, thereby slowing or preventing the flow of materials through the passageway. Obstruction in body passageways that are affected by cancer are not only in and of themselves life-threatening, they also limit the quality of a patient's life.

The primary treatment for the majority of tumors which cause neoplastic obstruction is surgical removal and/or chemotherapy, radiation therapy or laser therapy. Frequently a tumor causing an obstruction in a body passageway is inoperable and generally will not respond to traditional therapies. One approach to this problem has been the insertion of endoluminal stents. Briefly, stents are devices placed into the lumen of a body passageway to physically hold open a passageway that has been blocked by a tumor or other tissues/substances. Representative examples of commonly deployed stents include the Wallstent, Stecker stent, Gianturco stent and Palmaz stent (see for example, U.S. Pat. Nos. 5,102,417, 5,195,984, 5,176,626, 5,147,370, 5,141,516, 4,776,337). The metallic stents are frequently ineffective long term as the tumor is often able to grow into the lumen through the interstices of the stent. Stents in the lumen can also induce the ingrowth of reactive or inflammatory tissue onto the surface of the stent. The result is re-blockage of the body passageway which the stent was inserted to correct.

Other diseases, which although not neoplastic nevertheless involve proliferation, can likewise obstruct body passageways. For example, narrowing of the prostatic urethra due to benign prostatic hyperplasia is a serious problem affecting 60% of all men over the age of 60 years of age and 100% of all men over the age of 80 years of age. Present pharmacological treatments, such as 5-alphareductase inhibitors (for example Finasteride), or alpha-adrenergic blockers (for example, Terazozan) are generally only effective in a limited population of patients.

Moreover, of the surgical procedures that can be performed (for example, trans-urethral resection of the prostate (TURPs); open prostatectomy, or endo-urologic procedures such as laser prostatectomy, use of microwaves, hypothermia, cryosurgery or stenting), numerous complications such as bleeding, infection, incontinence, impotence, and recurrent disease, typically result.

In addition to neoplastic or proliferative diseases, other diseases such as vascular disease can result in the narrowing, weakening and/or obstruction of body passageways. According to 2004 estimates (source—U.S. American Heart Association), about 62 million Americans have one or more forms of cardiovascular disease. These diseases claimed about 950,000 lives in the same year (40% of all deaths in the United States.

Balloon angioplasty (with or without stenting) is one of the most widely used treatments for vascular disease; other options such as laser angioplasty are also available. While this is the treatment of choice in many cases of severe narrowing of the vasculature, about one-third of patients undergoing balloon angioplasty (source Heart and Stoke Foundation homepage) have renewed narrowing of the treated arteries (restenosis) within 6 months of the initial procedure; often serious enough to necessitate further interventions.

Such vascular diseases (including for example, restenosis) are due at least in part to intimal thickening secondary to vascular smooth muscle cell (VSMC) migration, VSMC proliferation, and extra-cellular matrix deposition. Briefly, vascular endothelium acts as a nonthrombogenic surface over which blood can flow smoothly and as a barrier which separates the blood components from the tissues comprising the vessel wall. Endothelial cells also release heparin sulphate, prostacyclin, EDRF and other factors that inhibit platelet and white cell adhesion, VSMC contraction, VSMC migration and VSMC proliferation. Any loss or damage to the endothelium, such as occurs during balloon angioplasty, atherectomy, or stent insertion, can result in platelet adhesion, platelet aggregation and thrombus formation. Activated platelets can release substances that produce vasoconstriction (serotonin and thromboxane) and/or promote VSMC migration and proliferation (PDGF, epidermal growth factor, TGF-.beta., and heparinase). Tissue factors released by the arteries stimulates clot formation resulting in a fibrin matrix into which smooth muscle cells can migrate and proliferate.

This cascade of events leads to the transformation of vascular smooth muscle cells from a contractile to a secretory phenotype. Angioplasty induced cell lysis and matrix destruction results in local release of basic fibroblast growth factor (bFGF) which in turn stimulates VSMC proliferation directly and indirectly through the induction of PDGF production. In addition to PDGF and bFGF, VSMC proliferation is also stimulated by platelet released EGF and insulin-like growth factor-1.

Vascular smooth muscle cells are also induced to migrate into the media and intima of the vessel. This is enabled by release and activation of matrix metalloproteases which degrade a pathway for the VSMC through the extra-cellular matrix and internal elastic lamina of the vessel wall. After migration and proliferation the vascular smooth muscle cells then deposit an extra-cellular matrix consisting of gylcosaminoglycans, elastin and collagen which comprises the largest part of intimal thickening. A significant portion of the restenosis process may be due to remodeling of the vascular wall leading to changes in the overall size of the artery; at least some of which is secondary to proliferation within the adventitia (in addition to the media). The net result of these processes is a recurrence of the narrowing of the vascular wall which is often severe enough to require a repeat intervention.

In summary, virtually any forceful manipulation within the lumen of a blood vessel or a non-vascular lumen will damage or denude its endothelial or epithelial lining. Thus, treatment options for vascular or non-vascular diseases themselves and for restenosis following therapeutic interventions continue to be major problems with respect to longterm outcomes for such conditions.

In addition to neoplastic obstructions and vascular disease, there are also a number of acute and chronic inflammatory diseases which result in obstructions of body passages. These include, for example, vasculitis, gastrointestinal tract diseases (for example Crohn's disease, ulcerative colitis) and respiratory tract diseases (for example asthma, chronic obstructive pulmonary disease).

Each of these diseases can be treated, to varying degrees of success, with medications such as anti-inflammatories or immunosuppressants. Current regimens however are often ineffective at slowing the progression of disease, and can result in systemic toxicity and undesirable side effects. Surgical procedures can also be utilized instead of or in addition to medication regimens. Such surgical procedures however have a high rate of local recurrence to due to scar formation, and can under certain conditions (for example, using balloon catheters), result in benign reactive overgrowth.

Other diseases that can also obstruct body passageways include infectious diseases. Briefly, there are a number of acute and chronic infectious processes that can result in the obstruction of body passageways including for example, urethritis, prostatitis and other diseases of the male reproductive tract, various diseases of the female reproductive tract, cystitis and urethritis (diseases of the urinary tract), chronic bronchitis, tuberculosis and other mycobacteria infections and other respiratory problems and certain cardiovascular diseases.

Such diseases are presently treated either by a variety of different therapeutic regimens and/or by surgical procedures. As above however, such therapeutic regimens have the difficulty of associated systemic toxicity that can result in undesired side effects. In addition, as discussed above surgical procedures can result in local recurrence due to scar formation, and in certain procedures (for example, insertion of commercially available stents), may result in benign reactive overgrowth.

Currently there are no tracheo-bronchial drug-eluting stents (DES). There are device companies such as Boston Scientific, Cook International and Alveolus that develop tracheo-bronchial stents. Other interventional procedures include laser, endobronchial electrosurgery, brachytherapy, photodynamic therapy and cryotherapy. Among these laser and endobronchial electro surgery (electrocautery) provide immediate palliation and are more frequently used. However these could cause tissue necrosis of the highly vascularized lung tumors which could lead to significant hemorrhage, enhanced morbidity and mortality. Brachytherapy, cryotherapy, and photodynamic therapy (PDT) do not provide immediate palliation and restoration of airway patency.

The existing treatments for the above diseases and conditions for the most part share the same limitations. The use of therapeutic agents have not resulted in the reversal of these conditions and whenever an intervention is used to treat the conditions, there is a risk to the patient as a result of the body's response to the intervention. The present invention provides compositions and methods suitable for treating the conditions and diseases which are generally discussed above. These compositions and methods address the problems associated with the existing procedures, offer significant advantages when compared to existing procedures, and in addition, provide other, related advantages.

### DISCLOSURE OF THE INVENTION

The invention provides a drug delivery system and methods of using said system to treat and prevent an obstruction in a body passageway. The invention has utility for in the treatment of and prevention of cancers, benign tumors, and hyperplasia.

In one embodiment the invention provides a drug delivery system, the drug delivery system comprising a support, a first polymer matrix, and a drug. In a preferred embodiment the first polymer matrix comprises a first material that is substantially susceptible to degradation by a composition having biological enzyme activity. In an alternative embodiment the first polymer matrix comprises a second material that is substantially resistant to degradation by a composition having biological enzyme activity. In a second alternative embodiment the first polymer matrix comprises one first material and one second material, wherein the first material is substantially susceptible to degradation by a composition having biological enzyme activity and the second material is substantially resistant to degradation by a composition having biological enzyme activity.

In another embodiment the invention provides the drug delivery system herein disclosed wherein the support is selected from the group consisting of a stent, a balloon, and a second polymer matrix. In a preferred embodiment the stent is selected from the group consisting of a tubular structure, a metallic self-expanding stent, a balloon expandable metallic stent, a self-expanding stent, and a stent-graft.

In another preferred embodiment the second polymer matrix comprises a first material that is substantially susceptible to degradation by a composition having biological enzyme activity. In another alternative embodiment, the second polymer matrix comprises a second material that is substantially resistant to degradation by a composition having biological enzyme activity. In a yet further preferred embodiment, the second polymer matrix comprises one first material and one second material, wherein the first material is substantially susceptible to degradation by a composition having biological enzyme activity and the second material is substantially resistant to degradation by a composition having biological enzyme activity.

The invention also provides the drug delivery system as disclosed herein wherein the first polymer matrix comprises a composition selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof. In one preferred embodiment, the first polymer matrix comprises a copolymer together with monomers of a hydrophilic polymer selected from the group consisting of polyvinylpyrrolidone, polyvinylalcohol, polyhydroxyethylmethacrylate, polyacrylamide, polymethacrylamide, and polyethyleneglycol.

In the alternative, the drug delivery system comprises a second polymer matrix wherein the second polymer matrix comprises a composition selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof.

In one preferred embodiment, the second polymer matrix comprises a copolymer together with monomers of a hydrophilic polymer selected from the group consisting of polyvinylpyrrolidone, polyvinylalcohol, polyhydroxyethylmethacrylate, polyacrylamide, polymethacrylamide, and polyethyleneglycol.

The invention also provides the drug delivery system as disclosed herein further comprising a pharmaceutical formulation. In a preferred embodiment the pharmaceutical formulation comprises the drug and a suitable pharmaceutical carrier.

The invention further provides the drug delivery system as disclosed herein wherein the drug is selected from the group consisting of thalidomide, docetaxel, etoposide, irinotecan, paclitaxel, teniposide, topotecan, vinblastine, vincristine, vindesine, busulfan, improsulfan, piposulfan, aziridines, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, temozolomide, aclacinomycinsa actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin, denopterin, edatrexate, methotrexate, piritrexim, pteropterin, TOMUDEX, trimetrexate, cladribine, fludarabine, 6-mercaptopurine, thiamiprine, thioguanine, ancitabine, azacitidine, 6-azauridine, carmofur, cytarabine, doxifluridine, emitefur, enocitabune, floxuridine, fluorouracil, gemcitabine, tegafur, L-asparaginase, interferon-α, interferon-13, interferon-γ, interleukin-2, lentinan, propagermanium, PSK, roquinimex, sizofican, ubenimex, carboplatin, cisplatin, miboplatin, oxaliplatin, aceglarone, amsacrine, bisantrene, defosfamide, demecolcine, diaziquone, eflomithine, elliptinium acetate, etoglucid, fenretinide, gallium nitrate, hydroxyurea, lonidamine, miltefosine, mitoguazone, mitoxantrone, mopidamol, nitracine, pentostain, phenamet, podophyllinic acid 2-ethyl-hydrazide, procabazine, razoxane, sobuzoxane, spirogermanium, tenuzonic acid, triaziquone, 2,2′,2″trichlorotriethylamine, urethan, calusterone, dromostanolone, epitiostanol, mepitiostane, testolacone, aminoglutethimide, mitotane, trilostane, bicalutamide, flutamide, nilutamide, droloxifene, tamoxifen, toremifene, aminoglutethimide, anastrozole, fadrozole, formestane, letrozole, fosfestrol, hexestrol, polyestradiol phosphate, buserelin, goserelin, leuprolide, triptorelin, chlormadinone acetate, medroxyprogesterone, megestrol acetate, melengestrol, porfimer sodium, batimastar, folinic acid, salicylates, salsalate, mesalamine, diflunisal, choline magnesium trisalicylate, diclofenac, diflunisal, etodolac, fenoprofen, flurbiprofen, ibuprofen, indomethacin, mefenamic acid, nabumetone, naproxen, piroxicam, phenylbutazone, ketoprofen, S-ketoprofen, ketorolac tromethamine, sulindac, tolmetin, beclomethasone, betamethasone, cortisone, dexamethasone, fluocinolone, flunisolide, fluticasone proprionate, fluorinated-corticoids, triamcinolone-diacetate, hydrorcortisone, clobetasol, prednisolone, methylprednisolone, prednisone, finasteride, adenocorticosteroids, cyclosporin, rapamycin, everolimus, sutinib maleate, gefitinib, erlotinib.

In one embodiment the stent comprises a metal selected from the group consisting of nickel-titanium alloy, chromel, stainless steel, copper, gold, platinum, silver, and titanium. In an alternative embodiment the stent comprises a material selected from the group consisting of conductive epoxy, conductive polymers, barium sulfate, titanium oxide, silicone, polyurethane, polyethylene, acrylonitrile butadiene styrene, polycarbonate, polypropylene, styrene, polyamide, polyimide, PEEK, PEBAX, polyester, PVC, fluoropolymers, and co-polymers thereof.

The invention provides a method for treating obstruction of a body passageway in a subject, the method comprising the steps of: (i) providing a first polymer matrix, the first polymer matrix comprising a drug, wherein the first polymer matrix is in a phase suitable for placing in the body passageway and wherein the first polymer matrix comprises a compound that allows the drug to elute from the first polymer matrix; (ii) introducing the first polymer matrix into the body passageway proximal to the obstruction; (iii) allowing the drug to be eluted from the first polymer matrix to a vicinity adjacent to the obstruction, the drug thereby effecting biological activity upon the obstruction, the method resulting in treating the obstruction. In one preferred embodiment the body passageway is selected from the group consisting of coronary artery, carotid artery, aorta, pulmonary artery, vein, capillary, trachea, bronchus, bronchioles, oesaphagus, bile duct, fallopian tubes, urethra, colon, bladder, pancreatic passageway, nasal passageways, male reproductive tract, female reproductive tract, small intestine, large intestine, cranial sinus, and brain sinus. In another preferred embodiment the first polymer matrix comprises a polymer selected from the group consisting of bio-degradable polymers, non-bio-degradable polymers, and combinations thereof. In another preferred embodiment the phase of the first polymer matrix is selected from the group consisting of a liquid, a gel, a solid, and combinations thereof. In an yet further alternative preferred embodiment the first polymer matrix comprises a polymer selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof. In a preferred embodiment the drug is selected from the group consisting of thalidomide, docetaxel, etoposide, irinotecan, paclitaxel, teniposide, topotecan, vinblastine, vincristine, vindesine, busulfan, improsulfan, piposulfan, aziridines, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, temozolomide, aclacinomycinsa actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin, denopterin, edatrexate, methotrexate, piritrexim, pteropterin, TOMUDEX, trimetrexate, cladribine, fludarabine, 6-mercaptopurine, thiamiprine, thioguanine, ancitabine, azacitidine, 6-azauridine, carmofur, cytarabine, doxifluridine, emitefur, enocitabune, floxuridine, fluorouracil, gemcitabine, tegafur, L-asparaginase, interferon-α, interferon-13, interferon-γ, interleukin-2, lentinan, propagermanium, PSK, roquinimex, sizofican, ubenimex, carboplatin, cisplatin, miboplatin, oxaliplatin, aceglarone, amsacrine, bisantrene, defosfamide, demecolcine, diaziquone, capecitabine, eflomithine, elliptinium acetate, etoglucid, fenretinide, gallium nitrate, hydroxyurea, lonidamine, miltefosine, mitoguazone, mitoxantrone, mopidamol, nitracine, pentostain, phenamet, podophyllinic acid 2-ethyl-hydrazide, procabazine, razoxane, sobuzoxane, spirogermanium, tenuzonic acid, triaziquone, 2,2′,2″trichlorotriethylamine, urethan, calusterone, dromostanolone, epitiostanol, mepitiostane, testolacone, aminoglutethimide, mitotane, trilostane, bicalutamide, flutamide, nilutamide, droloxifene, tamoxifen, toremifene, aminoglutethimide, anastrozole, fadrozole, formestane, letrozole, fosfestrol, hexestrol, polyestradiol phosphate, buserelin, goserelin, leuprolide, triptorelin, chlormadinone acetate, medroxyprogesterone, megestrol acetate, melengestrol, porfimer sodium, batimastar, folinic acid, salicylates, salsalate, mesalamine, diflunisal, choline magnesium trisalicylate, diclofenac, diflunisal, etodolac, fenoprofen, flurbiprofen, ibuprofen, indomethacin, mefenamic acid, nabumetone, naproxen, piroxicam, phenylbutazone, ketoprofen, S-ketoprofen, ketorolac tromethamine, sulindac, tolmetin, beclomethasone, betamethasone, cortisone, dexamethasone, fluocinolone, flunisolide, fluticasone proprionate, fluorinated-corticoids, triamcinolone-diacetate, hydrorcortisone, clobetasol, prednisolone, methylprednisolone, prednisone, finasteride, adenocorticosteroids, cyclosporin, rapamycin, everolimus, sutinib maleate, gefitinib, erlotinib. In another preferred embodiment, the first polymer matrix is introduced into the body passageway in combination with a support. In a more preferred embodiment the support is selected from the group consisting of a stent, a balloon, and a second polymer matrix. In yet more preferred embodiment the stent is selected from the group consisting of a tubular structure, a metallic self-expanding stent, a balloon expandable metallic stent, a self-expanding stent, and a stent-graft. In another preferred embodiment, the stent comprises a metal selected from the group consisting of nickel-titanium alloy, chromel, stainless steel, copper, gold, platinum, silver, and titanium. In a still further preferred embodiment, the second polymer matrix comprises a polymer selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof. In a most preferred embodiment the obstruction is selected from the group consisting of a tumor, vascular smooth muscle, endothelium, extracellular matrix, platelet aggregate, a thrombus, fibrin matrix, epidermal tissue, and neurological tissue.

The invention also provides a use of a composition as disclosed herein, the composition comprising a support, a first polymer matrix, and a drug for the manufacture of a device for the treatment of an obstruction in a body passageway.

The invention also provides a drug delivery system as disclosed herein, the drug delivery system for use in the treatment of or prevention of an obstruction in a body passageway, the drug delivery system comprising a support, a first polymer matrix, a fluorescent dye, and a drug.

The invention also provides a method for treating obstruction of or preventing vascular or non-vascular diseases associated with body passageways, comprising delivering locally through a polymer matrix from an implanted device to the body passageway a therapeutic agent from a class of agents including, a tyrosine kinase inhibitor, anti-neoplastics, anti-proliferative agents, anti-inflammatory agents, cytoprotectant, antibiotics, chemotherapeutics, antivirals, targeting compounds, cortico-steroids, cytokines, immunotoxins, anti-tumor antibodies, anti-angiogenic agents, anti-edema agents, radiosensitizers, and combinations thereof.

In one embodiment the method comprises delivering to the body passageway composition comprising thalidomide or an analogue or derivative thereof, neomycin, or an analogue or derivative thereof or paclitaxel or an analogue or derivative thereof or combinations thereof.

In another embodiment the method comprises delivering to the body passageway a composition comprising thalidomide or an analogue or derivative thereof, neomycin, or an analogue or derivative thereof or paclitaxel or an analogue or derivative thereof or a combination of the same through a locally implanted medical device.

In yet another embodiment the thalidomide or an analogue or derivative thereof, neomycin, or an analogue or derivative thereof or paclitaxel or an analogue or derivative thereof or combinations thereof further comprises a polymer coated or bound to the implanted medical device.

In a still further embodiment the polymer is a hydrophobic polymer selected from the group consisting of partially or completed esterified polymers or copolymers of acrylic or methacrylic acid, polyphosphazenes, polycarbonates, polylactic acid, polyglycolic acid, copolymers of lactic acid or glycolic acid, polyhydroxybutyric acid, polyorthoesters, polyanhydrides, polysiloxanes, polycaprolactone, polysaccharides, polyprotains and copolymers prepared from the monomers of these polymers.

In another embodiment the hydrophobic polymer is provided in the form of a copolymer together with monomers of a hydrophilic polymer selected from the group consisting of polyvinylpyrrolidone, polyvinylalcohol, polyhydroxyethylmethacrylate, polyacrylamide, polymethacrylamide, and polyethyleneglycol.

In a preferred embodiment the polymer is olefin polymers, polyethylene, polypropylene, polyvinyl chloride, polytetrafluoroethylene, polyvinyl acetate, polystyrene, poly(ethylene terephthalate), polyurethane, polyurea, silicone rubbers, polyamides, polycarbonates, polyaldehydes, natural rubbers, polyether-ester copolymers, styrene-butadiene copolymers and combinations thereof.

In another preferred embodiment the polymer is a copolymer of lactic acid and glycolic acid, poly(caprolactone), poly(lactic acid), poly(ethylene-vinyl acetate), gelatin, hyaluronic acid, chitosan, polyvinylalcohol, polyvinypyrrolidone or combinations thereof.

In another embodiment the polymer is a family of polyethylene glycol based ether-anhydride copolymers, such as polyethylene glycol-sebacic acid

In an alternative embodiment the polymer is a family of surface erodible polyanhydrides such as poly(carboxyphenoxy alkane-co-alkanoic acids)co-polymers, such as 1,6-bis-(carboxyphenoxy)hexane-cosebacic acid, and poly[1,3-bis(carboxyphenoxy)propane-co-sebacic-acid] or derivatives or combinations of these.

In another alternative embodiment the polymer is poly(hydroxy acids), polyanhydrides, polyorthoesters, polyphosphazenes, polyphosphates, polycaprolactone, polyhydroxybutyrates, polyesters, polyamides, polysaccharides, and polyproteins.

In another embodiment the thalidomide or an analogue or derivative thereof, neomycin, or an analogue or derivative thereof, curcumin or an analogue or derivative thereof, paclitaxel or an analogue or derivative thereof, further comprises other therapeutic agents such as thalidomide, docetaxel, etoposide, irinotecan, paclitaxel, teniposide, topotecan, vinblastine, vincristine, and vindesine, busulfan, improsulfan, piposulfan, aziridines, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, temozolomide, aclacinomycinsa actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin, denopterin, edatrexate, methotrexate, piritrexim, pteropterin, TOMUDEX, trimetrexate, cladribine, fludarabine, 6-mercaptopurine, thiamiprine, thioguanine, ancitabine, azacitidine, 6-azauridine, carmofur, cytarabine, doxifluridine, emitefur, enocitabune, floxuridine, fluorouracil, gemcitabine, tegafur, L-asparaginase, interferon-.alpha., interferon-.beta., interferon-.gamma., interleukin-2, lentinan, propagermanium, PSK, roquinimex, sizofican, ubenimex, carboplatin, cisplatin, miboplatin, oxaliplatin, aceglarone, amsacrine, bisantrene, defosfamide, demecolcine, diaziquone, eflomithine, elliptinium acetate, etoglucid, fenretinide, gallium nitrate, hydroxyurea, lonidamine, miltefosine, mitoguazone, mitoxantrone, mopidamol, nitracine, pentostain, phenamet, podophyllinic acid 2-ethyl-hydrazide, procabazine, razoxane, sobuzoxane, spirogermanium, tenuzonic acid, triaziquone, 2,2′,2″trichlorotriethylamine, urethan, calusterone, dromostanolone, epitiostanol, mepitiostane, testolacone, aminoglutethimide, mitotane, trilostane, bicalutamide, flutamide, nilutamide, droloxifene, tamoxifen, toremifene, aminoglutethimide, anastrozole, fadrozole, formestane, letrozole, fosfestrol, hexestrol, polyestradiol phosphate, buserelin, goserelin, leuprolide, triptorelin, chlormadinone acetate, medroxyprogesterone, megestrol acetate, melengestrol, porfimer sodium, batimastar, and folinic acid, nonsteroidal agents (“NSAIDS”) such as salicylates (e.g., salsalate, mesalamine, diflunisal, choline magnesium trisalicylate), diclofenac, diflunisal, etodolac, fenoprofen, flurbiprofen, ibuprofen, indomethacin, mefenamic acid, nabumetone, naproxen, piroxicam, phenylbutazone, ketoprofen, S-ketoprofen, ketorolac tromethamine, sulindac, tolmetin); other anti-inflammatory steroidal agents such as beclomethasone, betamethasone, cortisone, dexamethasone, fluocinolone, flunisolide, fluticasone proprionate, fluorinated-corticoids, triamcinolone-diacetate, hydrorcortisone, clobetasol, prednisolone, methylprednisolone and prednisone; immunosuppressive agents such as adenocorticosteroids, cyclosporin, rapamycin, everolimus, sutinib maleate, gefitinib, erlotinib or analogues or derivatives thereof. These therapeutic agents could be used in the absence of thalidomide or neomycin or clobetasol or curcumin.

In another embodiment the thalidomide or an analogue or derivative thereof, neomycin, or an analogue or derivative thereof, paclitaxel or an analogue or derivative thereof, further comprises other carriers such as cells, proteins, biological materials.

In another alternative embodiment the thalidomide or an analogue or derivative thereof, neomycin, or an analogue or derivative thereof, paclitaxel or an analogue or derivative thereof, is locally delivered to the passageway via a medically implanted device such as a bio-erodible or bio-absorbable or bio-degradable stent or tubular structure conforming to the passage, metallic self-expanding stent, balloon expandable metallic stent, self-expanding stent with polymer sheath or a polymer tube, or stent-graft.

In one embodiment the body passageway is a coronary artery, carotid artery, aorta, pulmonary artery, vein, capillary, trachea, bronchus, bronchioles, oesaphagus, bile duct, fallopian tubes, urethra, colon, bladder, pancreatic passageway, nasal passageways, male reproductive tract, female reproductive tract, small and large intestines.

In another embodiment the vascular or non-vascular disease is stenosis, restenosis, atherosclerosis, inflammation, angiogenesis, proliferation of local tissue, cancer, bacterial infection and combination thereof.

In a preferred embodiment the drug delivery system further comprises wherein the pair of drugs in combination are thalidominde and paclitaxel, wherein the first polymer matrix comprises a polyanhydride, wherein the fluorescent dye is indocyanine green, and wherein the support comprises NITINOL. In one embodiment the support further comprises nanoporous tantalum.

In another preferred embodiment the drug delivery system further comprises wherein the pair of drugs in combination are 5-fluorouracil and paclitaxel, wherein the first polymer matrix comprises a polyanhydride, wherein the fluorescent dye is indocyanine green, and wherein the support comprises NITINOL. In one embodiment the support further comprises nanoporous tantalum.

In another preferred embodiment the drug delivery system further comprises wherein the pair of drugs in combination are capecitabine and carmofur, wherein the first polymer matrix comprises a polyanhydride, wherein the fluorescent dye is indocyanine green, and wherein the support comprises NITINOL. In one embodiment the support further comprises nanoporous tantalum.

In a preferred embodiment the method further comprises wherein the pair of drugs in combination are thalidominde and paclitaxel, wherein the first polymer matrix comprises a polyanhydride, wherein the fluorescent dye is indocyanine green, and wherein the support comprises NITINOL. In one embodiment the support further comprises nanoporous tantalum.

In another preferred embodiment the method further comprises wherein the pair of drugs in combination are 5-fluorouracil and paclitaxel, wherein the first polymer matrix comprises a polyanhydride, wherein the fluorescent dye is indocyanine green, and wherein the support comprises NITINOL. In one embodiment the support further comprises nanoporous tantalum.

In a preferred embodiment the method further comprises wherein the pair of drugs in combination are capecitabine and carmofur, wherein the first polymer matrix comprises a polyanhydride, wherein the fluorescent dye is indocyanine green, and wherein the support comprises NITINOL. In one embodiment the support further comprises nanoporous tantalum.

The invention also provides a composition comprising a biodegradable polymer, wherein said biodegradable polymer is a blend of any of the polymers selected from the group of polymers consisting of poly(hydroxy acids), polyanhydrides, polyorthoesters, polyphosphazenes, polyphosphates, polycaprolactone, polyhydroxybutyrates, polyesters, polyamides, polysaccharides, and polyproteins.

### BRIEF DESCRIPTION OF THE DRAWINGS

FIG. 1 illustrates one exemplary embodiment of the invention, a rod- or cylinder-shaped system, showing a support 1, a first polymer matrix 2, and a drug 3. In this figure and those following, the polymer matrix and/or support and the drug may alternatively be a homogenous composition.

FIG. 2 illustrates one exemplary embodiment of the invention, a membranous or layered system, showing a support 1, a first polymer matrix 2, and a drug 3.

FIG. 3 illustrates one exemplary embodiment of the invention, a rod- or cylinder-shaped system, showing a first polymer matrix 2, a drug 3, and a second polymer matrix 4.

FIG. 4 illustrates one exemplary embodiment of the invention, a membranous or layered system, showing a first polymer matrix 2, a drug 3, and a second polymer matrix 4.

FIG. 5 illustrates one exemplary embodiment of the invention, a rod- or cylinder-shaped system, showing, a first polymer matrix 2, a drug 3, and a second polymer matrix 4. Note that the first and second polymer matrices are homogeneous, represented by the alternating bands of materials.

FIG. 6 illustrates one exemplary embodiment of the invention, a membranous or layered system, showing a first polymer matrix 2, a drug 3, and a second polymer matrix 4.

FIG. 7 illustrates one exemplary embodiment of the invention, a rod- or cylinder-shaped system, showing a first polymer matrix 2, a drug 3, and a metal or alloy 5.

FIG. 8 illustrates one exemplary embodiment of the invention, a membranous or layered system, showing a first polymer matrix 2, a drug 3, and a metal or alloy 5.

FIG. 9 illustrates one exemplary embodiment of the invention, a rod- or cylinder-shaped system, showing a support 1, a first polymer matrix 2, a second polymer matrix 4, a metal or alloy 5, and a pharmaceutical formulation 6. Note that, in this figure and any others, the polymer matrix and/or support and the pharmaceutical formulation may alternatively be a homogenous composition.

FIG. 10 illustrates one exemplary embodiment of the invention, a membranous or layered system, showing a support 1, a first polymer matrix 2, a second polymer matrix 4, a metal or alloy 5, and a pharmaceutical formulation 6.

FIG. 11 illustrates a three-quarter view of an exemplary stent of the invention wherein the stent is a bio-erodable stent, a non-bio-erodable stent, or a stent-graft. The stent comprises a metal or alloy 5 with optional anchoring fins 7 and optional markers 8 thereupon, an optional lining 9 having drainage apertures 10. The stent metal or alloy is overlain by the polymer matrix or matrices and the drug and/or pharmaceutical formulation.

FIG. 12 illustrates an exemplary generic formulation process for developing and/or manufacturing a drug-eluting stent product. Process using BMS is process A and process using polymer drug is process B. The stent used is either a metal or metal alloy, biodegradable polymeric stent or hybrid stent. The bio-degradable stent may be manufactured by directly extruding drug(s)-polymer(s) formulations (process B) into a desired form shaped and adapted for a use using methods and techniques well known to those of skill in the art.

FIG. 13 illustrates repeat units of biodegradable polyanhydrides used in the invention; a) poly(SA), b) poly(CPH), and c) poly(CPTEG). In this figure, ‘m’ and ‘n’ represent the number of repeating units of each monomer.

FIG. 14 illustrates three main types of malignant tracheal obstruction and various bronchoscopic techniques. Explanation of symbols: +++=potentially superior clinical outcome; ++=potentially excellent clinical outcome; +=potentially good clinical outcome; 0=potentially poor clinical outcome; EBES=endobronchial electrosurgery; PDT=photodynamic therapy.

FIG. 15 illustrates one embodiment of a multi-functional theranostic delivery stent (MTDS) of the invention.

FIG. 16 illustrates the efficacy of a multi-functional drug-delivery stent (MDDS); Tumor growth curve in minimal residual tumor model.

FIG. 17 illustrates the efficacy of MDDS; Tumor growth curve in palpable tumor model.

FIG. 18 illustrates the structure of paclitaxel (PTX); Molecular formula C₄₇H₅₁NO₁₄.

FIG. 19 illustrates the structure of thalidomide (THX); Molecular formula of C₁₃H₁₀N₂O₄).

FIG. 20 illustrates the structure of indocyanine green (ICG).

FIG. 21 shows fast, medium and slow release profiles for HCFU, PTX, and ICG, respectively.

FIG. 22 shows whole body near infra-red fluorescence image of tumor-bearing mouse implanted with ICG coated ETDS

FIG. 23 shows tumor growth inhibition on Day 29 (n=8, p=0.01) for PTX-FLX ETDS and PTX-THX ETDS. At implantation day all groups received a single dose of PTX intra-peritoneally (5 mg/kg).

FIG. 24 shows tumor growth inhibition for PTX-FLX ETDS and PTX-HCFU ETDS At implantation day all groups received a single dose of PTX intra-peritoneally (5 mg/kg). (n=8 enrolled, mice with tumor size>2800 were terminated during study).

### MODES FOR CARRYING OUT THE INVENTION

Prior to setting forth the invention, it may be helpful to an understanding thereof to set forth definitions of certain terms that will be used hereinafter.

As used herein and in the appended claims, the singular forms “a,” “an,” and “the” include plural reference unless the context clearly dictates otherwise. Thus, for example, a reference to “a polymer” includes a plurality of such polymers, and a reference to “a drug” is a reference to one or more drugs and equivalents thereof, and so forth.

“Body passageway” as used herein refers to any of number of passageways, tubes, pipes, tracts, canals, sinuses or conduits which have an inner lumen and allow the flow of materials within the body. Representative examples of body passageways include arteries and veins, lacrimal ducts, the trachea, bronchi, bronchiole, nasal passages (including the sinuses) and other airways, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina and other passageways of the female reproductive tract, the vasdeferens and other passageways of the male reproductive tract, and the ventricular system (cerebrospinal fluid) of the brain and the spinal cord.

“Therapeutic agent” as used herein refers to those agents which can mitigate, treat, cure, or prevent a given disease or condition. Representative examples of therapeutic agents are discussed in more detail below, and include, for example, anti-angiogenic agents, anti-proliferative agents, anti-inflammatory agents, and antibiotics.

As noted above, the present invention provides methods for treating or preventing diseases associated with body passageways, comprising the step of delivering endoluminally to the body passageway a composition comprising a therapeutic agent, and within preferred embodiments, a compositions comprising a therapeutic agent and a polymeric carrier via a durable or bio-erodable tube, or stent-graft.

Briefly stated, the present invention provides methods for treating or preventing diseases associated with vascular and non-vascular body passageways, comprising the step of delivering to a body passageway a therapeutic agent via a medically implanted device such as a bio-erodible stent, stent, stent-graft, polymeric tubes. Within a related aspect, methods for treating or preventing diseases associated with body passageways are provided comprising the step of locally delivering a therapeutic agent endoluminally to the body passageway, via the medical device implant. By delivering the therapeutic compound locally to the site of disease, systemic and unwanted side effects can be avoided and total dosages can potentially be reduced.

Esophageal cancer is a deadly disease with limited treatment options. In addition to modest effects, toxic chemo-therapy drugs cripple patients with debilitating side-effects. The Esophageal Theranostic Delivery System (ETDS) disclosed herein addresses this clinical need with a multi-modal solution. It delivers a theranostic combination of chemotherapy drugs and a fluorescent dye directly to the cancer. The combination is loaded on a nano-porous surface on the ETDS. The physical scaffold props open any obstruction, the combination drugs treat the cancer and the fluorescent dye lights up the tumor, allowing monitoring of the treatment. The ETDS is projected to provide acute relief in from dysphagia (difficulty swallowing), enhance therapeutic effectiveness, reduce systemic toxicity and improve quality of life. More importantly, in combination with other therapies, it has the potential to prolong life. It is a platform technology and is being developed to treat other cancers.

The loco-regional, multi-functional theranostic-delivery system (MTDS) to treat lung and gastrointestinal cancer in patients with focal disease as an adjunct to systemic therapy. The MTDS releases a combination of cancer chemo-therapeutic drugs and a fluorescent dye. The theranostic combination is loaded on a nitinol stent platform with a nano-porous, radio-opaque, Tantalum coat. It has multiple modes of action and:

(i) Serves as a physical platform to prop open tumor-related obstruction and provide acute relief from obstruction.
(ii) Enhances the therapeutic effectiveness by locally delivering clinically proven combination cancer chemotherapeutic drugs, Paclitaxel and Thalidomide and prevent re-obstruction of the due to tumor re-growth.
(iii) For synergistic activity or to reduce drug-resistance and/or tumor mutations, several others such as Epigenetic regulators and/or BET inhibitors (Huntley 2012) may be added on to the combination. These include for eg. DNA methyltransferase (epigenetic writers) inhibitors, azacytidine and decitabine, or histone deacetylase (epigenetic erasers) inhibitors, vorinostat and romidepsin, or BET (Bromodomain) inhibitors and/or chromatin regulators (epigenetic readers) e.g JQ1, IBET151, GSK525762, CPI-0610, TEN010 etc.
(iv) Diagnoses and monitors effectiveness of treatment by locally delivering fluorescent dye, Indocyanine Green, which lights up the tumor vasculature.
(v) An adjunct to systemic therapy the system has the potential to prolong survival.

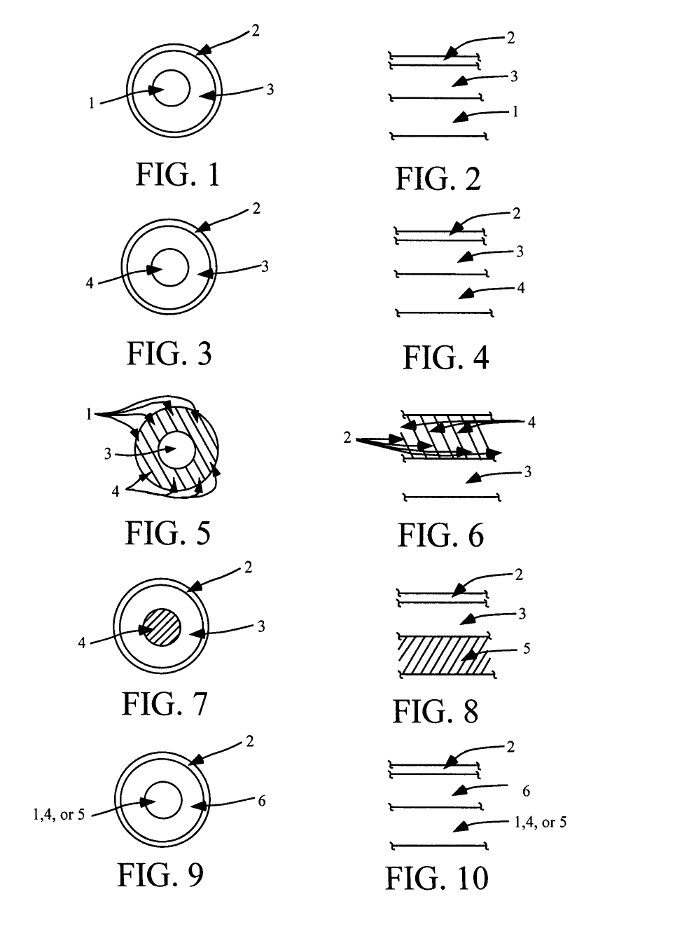

FIG. 1 illustrates an exemplary multi-functional theranostic delivery stent (MTDS):
1. Self-expanding metallic stent with a nano-porous high-surface Tantalumlayer (illustrated version);
2. Combination anti-cancer drugs and fluorescent dye embedded in the nano-porous Tantalum layer;
3. The implanted MTDS props open blocked airways and releases a cock tail of drugs-dye at an engineered rate to the tumor tissue.
   Tumor Visualization with Near Infra-Red Fluorescence Imaging (ICG) Along with GFP Fluorescent Tag:

Near infra-red fluorescence imaging, using an IVIS Imaging System 50 Series (Anticancer Inc., Xenogen, Alameda, Calif., USA) will be used. The optical imager is an integrated fluorescence system (400-900 nm) composed of a light-tight specimen chamber (dark box) and a 0.5 inch charge-coupled device (CCD) camera. To minimize electronic background and maximize sensitivity, the CCD camera is thermoelectrically cooled to −70° C. Fluorescence images of the animals will be acquired using the filter setting pre-set for Indocyanine Green with a background wavelength at 665-695 nm, an excitation wavelength at 710-760 μm, and an emission wavelength set at 810-875 nm. The GFP (green fluorescent protein) tagged tumor will also be monitored at an excitation wavelength at 395 nm, and an emission wavelength set at 509 nm.

### Related Research or R&D.

a. Rationale for Enhanced Therapeutic Effectiveness of the MDDS: Published Results and Our Own Preliminary Outlined Below Support Enhanced Therapeutic Effectiveness of MDDS.
i. Compelling Clinical Evidence of Enhanced Therapeutic Benefits from Local Therapy:

Loco-regional cancer chemotherapy provides high local concentrations, prolonged drug residence times, and has been shown to provide a pharmacokinetic advantage. It reduces dose-limiting toxicities and enhances therapeutic effectiveness. Successful loco-regional chemotherapy including site-specific endobronchial-intratumoral chemotherapy have proven providing effective neoadjuvant therapy for the management of lung cancer.²⁵⁻³¹ Clinical trials with or without systemic therapies have shown evidence of local therapy providing increased palliation, tumor control, increased QOL and some improving survival.^{30,31} For example, as an adjunct to surgery and radiation, Gliadel wafer, an FDA approved polymeric implant for gliobastomas (brain cancer), has increased median survival from 13.9 months to 11.6 months and is now the standard of care.³⁰ Clinical trials in esophageal cancer patients treated with brachytherapy stents loaded with 1-125 seeds, showed increased survival benefits (7 months vs. 4 months) and decreased dysphagia grades.³¹

### II. Preclinical Data Demonstrates POC Efficacy:

A Prototype Tracheo-Bronchial MDDS has been Developed:

Prototype tracheo-bronchial MDDS coated with biodegradable polymeric formulations of chemo-therapeutic agents paclitaxel (PTX), thalidomide (THX) and PTX-THX combinations with a drug load of 1-15 mg/MDDS with a release profile engineered from 1 to 3 months have been developed.

Tantalum Coated Nitinol Surface:

A 10-25 fold increase in drug-loading capacity of the Nitinol has been obtained with the nano-porous Tantalum coating. The increase was demonstrated on a 10×10 mm Nitinol coupon with and without the polymer.

Increased Aqueous Stability ICG:

Embedding ICG formulation on the Tantalum coated nanoporous surface has increased the aqueous stability of ICG. A ICG formulation on a 10×10 mm Nitinol coupon was demonstrated to be stable and releasing ICG over a 1 month period in an aqueous media.

Projections for Tantalum Coated MDDS:

Based on the drug loading and release profile obtained using the Tantalum coated nitinol coupon, it is estimated that the MDDS can be coated with a high drug load in the range of 25-100 mg/unit or higher with a release profile that can be engineered from 1 month to 3 months.

Demonstrated Loco-Regional Drug Distribution:

Obtained evidence of gradient loco-regional drug distribution in the tracheo-bronchial and lung tissue in multiple preclinical studies, upon local intra-tracheal (IT) delivery. Demonstrated a 7-times higher lung tissue concentration after IT delivery than when delivered systemically.

POC Efficacy at 1000 Fold Lower Dose:

A POC preclinical multi-functional drug-delivery stent (MDDS) was efficacious and inhibited tumor growth in two preclinical mouse xenograft models implanted with human lung cancer cells. Efficacy was seen at a lower (1-2 μg/Kg) than 1/1000th of the systemic dose. Coated (drugs) and uncoated MDDS were implanted inside a grown tumor or near a growing tumor in a mouse xenograft model. In both cases the MDDS was efficacious (FIGS. 16 and 17).

### Rationale for the Drug Combination, Paclitaxel (PTX) and Thalidomide (THX)

The rationale for the drug combination PTX and THX addresses three key areas—efficacy, safety, physico-chemical attributes.

Efficacy: Superiority of multi-drug therapy with complementary mechanism of action is standard therapy in cancer treatment. Two rationales address the combination of PTX and THX a) combination of a chemotherapeutic with an antiangiogenic agent that blocks VEGF can increase the effectiveness of chemotherapy⁴³⁻⁴⁵ and (b) Prolonged, low-dose, exposure to chemotherapeutic drugs (metronomic dosing) can induce anti-neoplasm effect through the anti-angiogenesis activity and this activity can be potentiated by an anti-angiogenic agent.⁴³⁻⁴⁵ PTX, a mitotic inhibitor and THX, an angiogenesis inhibitor, are multi-targeted compounds with complementary mechanisms of action that have separately demonstrated preclinical and clinical efficacy in lung or other cancers.⁴⁵⁻⁴⁸

Safety: No pulmonary toxicities have been seen with either PTX or THX when administered systemically.⁴⁵⁻⁴⁸

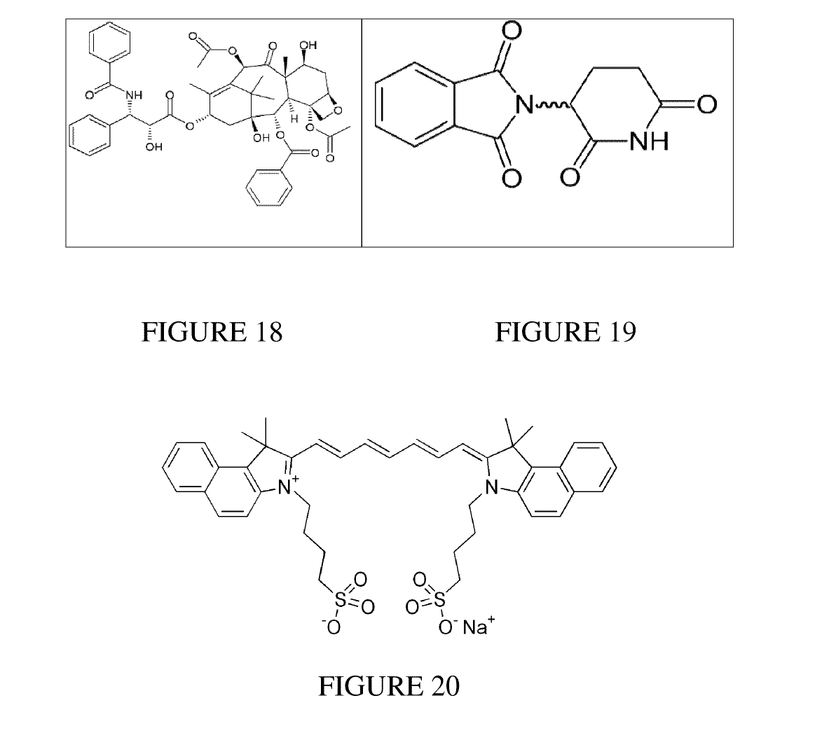

Desirable physico-chemical attributes of PTX and THX: The physico-chemical attributes of PTX and THX are desirable for local delivery, requiring prolonged residence time at delivery site. Both compounds are lipohilic, and amenable to our coating technology. The structures of PTX and THX are shown in FIGS. 18 and 19.

### Rationale for the Near Infra-Red Fluorescent Dye Indocyanine Green (ICG)

Indocyanine green (ICG)) is a safe, clinically approved optical imaging agent, ampiphilic tricarbocyanine near-infrared (NIR) dye with a molecular weight of 774.97 Da. FIG. 20 shows the chemical structure of ICG. ICG shows an absorbance peak at 805 nm and an emission peak at 830 nm in human plasma. This absorbance emission spectrum is within the near infrared range and is therefore, advantageous for optical imaging studies in vivo due to low background fluorescence. This can improve sensitivity, specificity and cost-effectiveness for early tumor detection.

The high in-vivo protein binding of ICG (about 95%), limits it largely to intravascular compartment and forms the basis of the majority of its applications [49-53] ICG has been used for diagnostic imaging of tumors, estimate tumor blood volume, cardiac output and the degree of vascularity in inflammatory processes [49-53] ICG was originally approved by the United States Food and Drug Administration (FDA) in 1956 for evaluation of the cardiovascular system and liver function, and more recently it has been approved for use in ophthalmic angiography [49-53].

ICG's shortcomings include its instability in aqueous media sensitivity to photo degradation and thermal degradation. Improving the stability and quantum yield of ICG would allow for more efficient imaging of disease processes, drug dosing and treatment effect.

We have stabilized ICC in the MTDS system. Embedding of ICG in the nanoporous Tantalum layer of the MTDS has improved the aqueous stability of ICG. As a stable source of loco-regional depot of NIR fluorescent imaging agent it has the potential of targeting the tumor vasculature over extended periods of time, effectively and efficiently imaging the therapeutic effectiveness of cancer treatment.

A wide variety of therapeutic agents may be utilized within the scope of the present invention, including for example anti-angiogenic agents, anti-proliferative agents, anti-inflammatory agents, antibiotics and combinations thereof.

Within certain embodiments of the invention, the therapeutic agents may further comprise a carrier (either polymeric or non-polymeric), such as, for example, poly(ethylene-vinyl acetate) (about 10%, 20%, 30%, 40%, 50%, 60%, 70%, 80%, 90%, or 100% crosslinked), copolymers of lactic acid and glycolic acid, poly(caprolactone), poly(lactic acid), copolymers of poly(lactic acid) and poly(caprolactone), gelatin, hyaluronic acid, collagen matrices, silicon, and albumin.

The therapeutic agents may be utilized to treat or prevent a wide variety of diseases, including for example, vascular diseases, neoplastic obstructions, inflammatory diseases and infectious diseases. Representative body passageways which may be treated include, for example, arteries, the esophagus, the stomach, the duodenum, the small intestine, the large intestine, biliary tracts, the ureter, the bladder, the urethra, lacrimal ducts, the trachea, bronchi, bronchioles, nasal airways, eustachian tubes, the external auditory canal, uterus and fallopian tubes.

Within one particularly preferred embodiment of the invention, the therapeutic agent is delivered endoluminally to the passageway via a medical device implant such as a durable or bio-erodible stent, polymer tube or stent-graft.

These and other aspects of the present invention will become evident upon reference to the following detailed description and attached drawings. In addition, various references are set forth below which describe in more detail certain procedures, devices or compositions, and are therefore incorporated by reference in their entirety.

As discussed in more detail below, a wide variety of therapeutic agents may be delivered to the body passageways, either with or without a carrier (for example, polymeric), in order to treat or prevent a disease associated with the body passageway. Each of these aspects is discussed in more detail below.

### Therapeutic Agents

As noted above, the present invention provides methods and compositions which utilize a wide variety of therapeutic agents. Within one aspect of the invention, the therapeutic agent is an antiangiogenic factor. Briefly, within the context of the present invention anti-angiogenic factors should be understood to include any protein, peptide, chemical, or other molecule which acts to inhibit vascular growth. A variety of methods may be readily utilized to determine the antiangiogenic activity of a given factor, including for example, chick chorioallantoic membrane (“CAM”) assays.

In addition to the CAM assay described above, a variety of other assays may also be utilized to determine the efficacy of anti-angiogenic factors in vivo, including for example, mouse models which have been developed for this purpose (see Roberston et al., (1991) Cancer Res. 51: 1339-1344).

A wide variety of anti-angiogenic factors may be readily utilized within the context of the present invention. Representative examples include Anti-Invasive Factor, retinoic acid and derivatives thereof, Suramin, Tissue Inhibitor of Metalloproteinase-1, Tissue Inhibitor of Metalloproteinase-2, Plasminogen Activator Inhibitor-1, Plasminogen Activator Inhibitor-2, compounds which disrupt microtubule function, and various forms of the lighter “d group” transition metals. These and other anti-angiogenic factors will be discussed in more detail below.

Briefly, Anti-Invasive Factor, or “AIF” which is prepared from extracts of cartilage, contains constituents which are responsible for inhibiting the growth of new blood vessels. These constituents comprise a family of 7 low molecular weight proteins (<50,000 Daltons; 50 kDa) (Kuettner and Pauli, “Inhibition of neovascularization by a cartilage factor” in Development of the Vascular System, Pitman Books (CIBA Foundation Symposium 100), pp. 163-173, 1983), including a variety of proteins which have inhibitory effects against a variety of proteases (Eisentein et al, Am. J. Pathol. 81:337-346, 1975; Langer et al., Science 193:70-72, 1976; and Horton et al., Science 199:1342-1345, 1978). AIF suitable for use within the present invention may be readily prepared utilizing techniques known in the art (for example, Eisentein et al, supra; Kuettner and Pauli, supra; and Langer et al., supra). Purified constituents of AIF such as Cartilage-Derived Inhibitor (“CDI”) (see Moses et al., Science 248:1408-1410, 1990) may also be readily prepared and utilized within the context of the present invention.

Retinoic acids alter the metabolism of extracellular matrix components, resulting in the inhibition of angiogenesis. Addition of proline analogs, angiostatic steroids, or heparin may be utilized in order to synergistically increase the anti-angiogenic effect of transretinoic acid. Retinoic acid, as well as derivatives thereof which may also be utilized in the context of the present invention, may be readily obtained from commercial sources, including for example, Sigma Chemical Co. (Sigma-Aldrich, St. Louis. Mo.; Cat. No. R2625).

Suramin is a polysulfonated naphthylurea compound that is typically used as a trypanocidal agent. Briefly, Suramin blocks the specific cell surface binding of various growth factors such as platelet derived growth factor (PDGF), epidermal growth factor (EGF), transforming growth factor β (TGF-β), insulin-like growth factor 1 (IGF-1), and β-fibroblast growth factor (βFGF). Suramin may be prepared in accordance with known techniques, or readily obtained from a variety of commercial sources, including for example Mobay Chemical Co., New York. (see Gagliardi et al., Cancer Res. 52:5073-5075, 1992; and Coffey, Jr., et al., J. Cell. Physiol. 132:143-148, 1987).

Tissue Inhibitor of Metalloproteinases-1 (TIMP-1) is secreted by endothelial cells which also secrete MMPases. TIMP-1 is glycosylated and has a molecular weight of 28.5 kDa. TIMP-1 regulates angiogenesis by binding to activated metalloproteinases, thereby suppressing the invasion of blood vessels into the extracellular matrix. Tissue Inhibitor of Metalloproteinases-2 (TIMP-2) may also be utilized to inhibit angiogenesis. Briefly, TIMP-2 is a 21 kDa nonglycosylated protein which binds to metalloproteinases in both the active and latent, proenzyme forms. Both TIMP-1 and TIMP-2 may be obtained from commercial sources such as Synergen, Boulder, Colo.

Plasminogen Activator Inhibitor-1 (PAI-1) is a 50 kDa glycoprotein which is present in blood platelets, and can also be synthesized by endothelial cells and muscle cells. PAI-1 inhibits tissue plasminogen activator (tPA) and urokinase plasminogen activator (uPA) at the basolateral site of the endothelium, and additionally regulates the fibrinolysis process. Plasminogen Activator Inhibitor-2 (PAI-2) is generally found only in the blood under certain circumstances such as in pregnancy, and in the presence of tumors. Briefly, PAI-2 is a 56 kDa protein which is secreted by monocytes and macrophages. It is believed to regulate fibrinolytic activity, and in particular inhibits urokinase plasminogen activator and tissue plasminogen activator, thereby preventing fibrinolysis.

Therapeutic agents of the present invention also include compounds which disrupt microtubule function. Representative examples of such compounds include estramustine (available from Sigma-Aldrich, St. Louis Mo.; Wang and Stearns Cancer Res. 48:6262-6271, 1988), epothilone, curacin-A, colchicine, methotrexate, and paclitaxel, vinblastine, vincristine, D.sub.20 and 4-tert-butyl-[3-(2-chloroethyl)ureido]benzene (tBCEU). Briefly, such compounds can act in several different manners. For example, compounds such as colchicine and vinblastine act by depolymerizing microtubules.

Within one preferred embodiment of the invention, the therapeutic agent is thalidomide, a compound which inhibits angiogenesis. The pharmaceutical composition of thalidomide could include, precursors, metabolites, derivatives and/or analogues of thalidomide.

Within one preferred embodiment of the invention, the therapeutic agent is Neomycin, a compound which inhibits angiogenesis and is anti-bacterial. The pharmaceutical composition of neomycin wherein the neomycin analogue is (a) neomycin A, neomycin B, or neomycin C; (b) a complex comprising neomycin A, neomycin B, or neomycin C; (c) an aminoglycoside having a structure substantially similar to that of neomycin A, neomycin B or neomycin C; (d) a chemical or biological breakdown product of neomycin A, neomycin B or neomycin C; (e) a derivative of neomycin A, neomycin B or neomycin C; or (f) a naturally-occurring precursor to neomycin A, neomycin B or neomycin C.

Other therapeutic agents that can be utilized within the present invention include a wide variety of antibiotics, including antibacterial, antimicrobial, antiviral, antiprotozoal and antifungal agents. Representative examples of such agents include systemic antibiotics such as aminoglycosides (for example, streptomycin, amikacin, gentamicin, netilmicin, tobramycin); 1st, 2nd, and 3rd generation cephalosporins (for example, cephalothin, cefazolin, cephapirin, cephradine, cephalexin, cefadroxil, cefaclor, cefamandole, cefuroxime, cefuroxime axetil, cefonicid, ceforanide, cefoxitin, cefotaxime, cefotetan, ceftizoxime, cefoperazone, ceftazidime, ceftriaxone, moxalactam, other semisynthetic cephalosporins such as cefixime and cefpodoxime proxetil); penicillins (for example, penicillin G (benzathine and procaine salts), cloxacillin, dicloxacillin, methicillin, nafcillin, oxacillin, penicillin V, ampicillin, amoxicillin, bacampicillin, cyclacillin, carbenicillin, ticarcillin, mezlocillin, piperacillin, azlocillin, amdinocillin, and penicillins combined with clavulanic acid); quinolones (for example, cinoxacin, ciprofloxacin, nalidixic acid, norfloxacin, pipemidic acid, perloxacin, fleroxacin, enoxacin, ofloxacin, tosufloxacin, lomefloxacin, stereoisomers of the quinolones); sulfonamides (for example, sulfacytine, sulfamethizole, sulfamethoxazole, sulfisoxazole, sulfasalazine, and trimethoprim plus sulfamethoxazole combinations); tetracyclines (for example, doxycycline, demeclocycline, methacycline, minocycline, oxytetracycline, tetracycline); macrolides (for example, erythromycins, other semisynthetic macrolides such as azithromycin and clarithromycin); monobactams (new synthetic class) (for example, aztreonam, loracarbef); and miscellaneous agents such as actinomycin D, doxorubicin, mitomycin C, novobiocin, plicamycin, rifampin, bleomycin, chloramphenicol, clindamycin, oleandomycin, kanamycin, lincomycin, neomycin, paromomycin, spectinomycin, troleandomycin, amphotericin B, colistin, nystatin, polymyxin B, griseofulvin, aztreonam, cycloserine, clindamycin, colistimethate, imipenem-cilastatin, methenamine, metronidazole, nitrofurantoin, rifabutan, spectinomycin, trimethoprim, bacitracin, vancomycin, other .beta.-lactam antibiotics.

Further therapeutic agents that can be utilized within the present invention include topical antibiotics such as bacitracin, zinc, mupirocin, clindamcin; antipathogenic polypeptides such as cecropionins, mangainins; and antitubercular agents such as sulfadimethoxine, sulfisoxazole, sulfisomidine, ethambutor hydrochloride, isoniazide, calcium paraminosalicylate.

Other therapeutic agents that can be utilized within the present invention include antibiotics such as iodine, povidone iodine, boric acid, sodium borate, oxydale, potassium permanganate, ethanol, isopropanol, formalin, cresol, dimazole, siccanin, phenyliodoundecynoate, hexachlorophene, resorcin, benzethonin chloride, sodium lauryl sulfate, mercuric chloride, mercurochrome, silver sulfadiazine and other inorganic and organic silver and zinc salts, salts of mono- and divalent cations, chlorhexidine gluconate, alkylpolyaminoethylglycine hydrochloride, benzalkonium chloride, nitrofurazone, nystatin, acesulfamin, clotrimazole, sulfamethizole, sulfacetamide, diolamine, tolnaftate, pyrrolnitrin, undecylenic acid, microazole, variotin, haloprogin, and dimazole, (meclocycline, trichomycin and pentamycin), penicillins. Antifungal agents include flucytosine, fluconazole, griseofluvin, ketoconazole and miconazole. Antiviral and AIDS agents include acyclovir, amantadine, didanosine (formerly ddI), griseofulvin, flucytosine, foscamet, ganciclovir, idoxuridine, miconazole, clotrimazole, pyrimethamine, ribavirin, rimantadine, stavudine (formerly d4T), trifluridine, trisulfapyrimidine, valacyclovir, vidarabine, zalcitabine (formerly ddC) and zidovudine (formerly AZT). Adjunct therapeutic agents for AIDS (for example, erythropoietin; fluconazole (antifungal); interferon α-2a and α-2b (Kaposi's sarcoma); atovaquone, pentamidine and trimetrexate (antiprotozoal); megestraol acetate (appetite enhancer); rifabutin (antimycobacterial). Representative examples of antiprotozoal agents include: pentamidine isethionate, quinine, chloroquine, and mefloquine.

Other therapeutic agents that can be utilized within the present invention include anti-proliferative, anti-neoplastic or chemotherapeutic agents. Representative examples of such agents include androgen inhibitors, antiestrogens and hormones such as flutamide, leuprolide, tamoxifen, estradiol, estramustine, megestrol, diethylstilbestrol, testolactone, goserelin, medroxyprogesterone; Cytotoxic agents such as altretamine, bleomycin, busulfan, carboplatin, carmustine (BiCNU), cisplantin, cladribine, dacarbazine, dactinomycin, daunorubicin, doxorubicin, estramustine, etoposide, lomustine, cyclophosphamide, cytarabine, hydroxyurea, idarubicin, interferon α-2a and -2b, ifosfamide, mitoxantrone, mitomycin, paclitaxel, streptozocin, teniposide, thiotepa, vinblastine, vincristine, vinorelbine; Antimetabolites and antimitotic agents such as floxuridine, 5-fluorouracil, fluarabine, interferon α-2a and α-2b, leucovorin, mercaptopurine, methotrexate, mitotane, plicamycin, thioguanine, colchicine, anthracyclines and other antibiotics, folate antagonists and other anti-metabolites, vinca alkaloids, nitrosoureas, DNA alkylating agents, purine antagonists and analogs, pyrimidine antagonists and analogs, alkyl sulfonates; enzymes such as asparaginase, pegaspargase; radioactive agents (for example, Cu-64, Ga-67, Ga-68, Zr-89, Ru-97, Tc-99m, Rh-105, Pd-109, In-111, I-123, I-125, I-131, Re-186, Re-188, Au-198, Au-199, Pb-203, At-211, Pb-212 and Bi-212), toxins (for example, ricin, abrin, diphtheria toxin, cholera toxin, gelonin, pokeweed antiviral protein, tritin, Shigella toxin, and Pseudomonas exotoxin A), adjunct therapeutic agents such as granisetron and ondansetron (antinauseants, antiemetics), dexrazoxane (cardiomyopathy), gallium nitrate (hypercalcemia), GCSF and GMSCF (chemotherapy and BMT), IL-1α, IL-2, IL-3, IL-4, levamisole, pilocarpine (saliva generation in radiation therapy setting), strontium 89 (bone tumors).

Further therapeutic agents that can be utilized within the present invention include cardiovascular agents; antihypertensive agents; adrenergic blockers and stimulators (for example, doxazosin, guanadrel, guanethidine, pheoxybenzamine, prazosin plus polythiazide, terazosin, methyldopa, clonidine, guanabenz, guanfacine); alpha-/beta-adrenergic blockers (for example, Labetalol); angiotensin converting enzyme (ACE) inhibitors (for example, benazepril, catopril, enalapril, enalaprilat, fosinopril, lisinopril, moexipril, quinapril, ramipril, and combinations with calcium channel blockers and diuretics; ACE-receptor antagonists (for example, losartan); beta blockers (for example, acebutolol, atenolol, betaxolol, bisoprolol, carteolol, esmolol, fimolol, pindolol, propranolol, penbatolol, metoprolol, nadolol, sotalol); calcium channel blockers (for example, amiloride, amlodipine, bepridil, diltiazem, isradipine, nifedipine, verapamil, felodipine, nicardipine, nimodipine); antiarrythmics, groups I-IV (for example, bretylium, disopyramide, encamide, flecamide, lidocaine, mexiletine, moricizine, propafenone, procainamide, quinidine, tocamide, esmolol, propranolol, acebutolol, amiodarone, sotalol, verapamil, diltiazem, pindolol, bupranolol hydrochloride, trichlormethiazide, furosemide, prazosin hydrochloride, metoprolol tartrate, carteolol hydrochloride, oxprenolol hydrochloride, and propranolol hydrochloride); and miscellaneous antiarrythmics and cardiotonics (for example, adenosine, digoxin; metildigoxin, caffeine, dopamine hydrochloride, dobutamine hydrochloride, octopamine hydrochloride, diprophylline, ubidecarenon, digitalis).

Other therapeutic agents that can be utilized within the present invention include diuretics (for example, acetazolamide, amiloride, triamterene plus hydrochlorothiazide combinations, spironolactone plus hydrochlorothiazide combinations, torsemide, furosemide, ethacrynate, bumetanide, triamterene, methylchorothizide, hydrochlorothiazide, metdazone, chlorthalidone, hydroflumethiazide, metolazone, methyclothiazide, polythiazide, quinithazone, trichlormethiazide, benroflumethiazide, benzthiazide); hypotensive diuretics (for example, mefruside, penflutizide, bumetamide, hydrothiazide, bentroflumethiazide, reserpine); Inotropic agents (for example, digoxin, digitoxin, dobutamine, aminone, milrinone); vasodilators (for example, papaverine, isosorbide mono- and dinitrates, nitroglycerin, dizoxide, hydralazine, minoxidil, nitroprusside, prazosin, terazosin, 1,2,3-propanetriolmononitrate, 1,2,3-propanetriolnitrate and their ester derivatives, pentaerythritol tetranitrate, hepronicate, molsidomine, nicomol, simfibrate, diltiazem hydrochloride, cinnarizine, dipyridamole, trapidil, trimetazidine hydrochloride, carbocromene, prenylamine lactate, dilazep dihydrochloride); vasopressors (for example, metaraminol, isoproterenol, phenylephrine, methaxamine); anticoagulant and thrombolytic agents (for example, tissue plasminogen activator (tPA), urokinase (uPA), streptokinase, pro-urokinase, urokinase, heparin, warfarin); calmodulin antagonists (for example, H7); inhibitors of the sodium/calcium antiporter (for example, Amiloride); and inhibitors of the ryanodine receptor (for example, Ryanodine); inhibitors of the inositol-3-phosphate (IP₃) receptor (for example, heparin).

Other therapeutic agents that can be utilized within the present invention include anti-inflammatory agents. Representative examples of such agents include nonsteroidal agents (NSAIDS) such as salicylates (for example, salsalate, mesalamine, diflunisal, choline magnesium trisalicylate), diclofenac, diflunisal, etodolac, fenoprofen, flurbiprofen, ibuprofen, indomethacin, mefenamic acid, nabumetone, naproxen, piroxicam, phenylbutazone, ketoprofen, S-ketoprofen, ketorolac tromethamine, sulindac, tolmetin). Other anti-inflammatory drugs include steroidal agents such as beclomethasone, betamethasone, cortisone, dexamethasone, fluocinolone, flunisolide, fluticasone proprionate, fluorinated-corticoids, triamcinolone-diacetate, hydrorcortisone, prednisolone, methylprednisolone and prednisone. Immunosuppressive agents (for example, adenocorticosteroids, cyclosporin); and antihistamines and decongestants (for example, astemizole (histamine H1-receptor antagonist), azatidine, brompheniramine, clemastine, chlorpheniramine, cromolyn, cyproheptadine, diphenylimidazole, diphenhydramine hydrochloride, hydroxyzine, glycyrrhetic acid, homochlorocyclizine hydrochloride, ketotifen, loratadine, naphazoline, phenindamine, pheniramine, promethazine, terfenadine, trimeprazine, tripelennamine, tranilast, and the decongestants phenylpropanolamine and pseudoephedrine.

Further therapeutic agents that can be utilized within the present invention include central nervous system agents. Representative examples of such agents include anti-depressants (for example, PROZAC, PAXIL, LUVOX, MANNEREX, and EFFEXOR); CNS stimulants (for example, pemoline, methamphetamine, dextroamphetamine); hypnotic agents (for example, pentobarbital, estazolam, ethchlorynol, flurazepam, propofol, secobarbital, temazepam, triazolam, quazepam, zolpidem tartrate); antimanic agents (for example, lithium); sedatives and anticonvulsant barbiturates (for example, pentobarbitol, phenobarbital, secobarbital, mephobarbital, butabarbital primidone, amobarbital); non-barbiturate sedatives (for example, diphehydramine, doxylamine, midazolam, diazepam, promethazine, lorazepam, temazepam); and other miscellaneous hypnotics and sedatives (for example, methaqualone, glutethimide, flurazepam, bromovalerylurea, flurazepam, hydrochloride, haloxazolam, triazolam, phenobarbital, chloral hydrate, nimetazepam, estazolam).

Other therapeutic agents that can be utilized within the present invention include, but are not limited to, tacrine (reversible cholinesterase inhibitor) for treating Alzhiemer's disease; for treament of Parkinson's disease, agents such as, but not limited to, amantadine, bromocriptine mesylate, biperiden, benztropine mesylate, carbidopa-levodopa, diphenhydramine, hyoscyamine, levodopa, pergolide mesylate, procyclidine, selegiline HCl, trihexyphenidyl HCl; and other miscellaneous CNS agents such as fluphenazine, flutazolam, phenobarbital, methylphenobarbital, thioridazine, diazepam, benzbromarone, clocapramine hydrochloride, clotiazepam, chlorpromazine, haloperidol, lithium carbonate.

Further therapeutic agents that can be utilized within the present invention include anti-migraine agents (for example, ergotamine, methylsergide, propranolol, dihydroergotamine, Sertroline and Immitrex); Post-cerebral embolism agents (for example, nicardipine hydrochloride, cinepazide maleate, pentoxifylline, ifenprodil tartrate); local anesthetics (for example, lidocaine, benzocaine, ethyl aminobenzoate, procaine hydrochloride, dibucaine, procaine; antiulcer/antireflux agents (for example, LOSEC (Omeprazole), aceglutamide aluminum, cetraxate hydrochloride, pirenzepine hydrochloride, cimetidine, famotidine, metoclopramide, ranitidine, L-glutamine, gefamate, and any stereoisomer of these compounds, and the pharmaceutically acceptable salts of these compounds, such compound used singly or in combination of more than one compound, properly chosen); protease inhibitors (for example, serine protease, metalloendoproteases and aspartyl proteases (such as HIV protease, renin, and cathepsin) and thiol protease inhibitors (for example, benzyloxycarbonyl-leu-norleucinal (calpeptin) and acetyl-leu-leu-norleucinal); phosphodiesterase inhibitors (for example, isobutyl methylxanthine); phenothiazines; growth factor receptor antagonists (for example, platelet-derived growth factor (PDGF), epidermal growth factor, interleukins, transforming growth factors alpha and beta, and acidic or basic fibroblast growth factors); antisense oligonucleotides (for example, sequences complementary to portions of mRNA encoding PDGF or other growth factors); and protein kinase inhibitors (for example, inhibitors of protein tyrosine kinases, protein kinase A, protein kinase C, protein kinase L, myosin light chain kinase, Ca²⁺/calmodulin kinase II, casein kinase II, RNA-activated protein kinase, mitogen-activated protein kinase, proliferation-related kinase, cyclin-dependent protein kinase, 5′-AMP-activated protein kinase);

Other therapeutic agents that can be utilized within the present invention include anti-tissue damage agents. Representative examples of such agents include superoxide dismutase; immune modulators (for example, lymphokines, monokines, interferon α, β, τ-1b, α-n3, α-2b; growth regulators (for example, IL-2, tumor necrosis factor, epithelial growth factor, somatrem, fibronectin, GM-CSF, CSF, platelet derived growth factor, somatotropin, rG-CSF, epidermal growth factor, IGF-1).

Other therapeutic agents that can be utilized within the present invention include monoclonal and polyclonal antibodies (for example, those active against: venoms, toxins, tumor necrosis factor, bacteria); hormones (for example, estrogen, progestin, testosterone, human growth hormone, epinephrine, levarterenol, thyroxine, thyroglobulin, oxytocin, vasopressin, ACTH, somatropin, thyrotropin, insulin, parathyrin, calcitonin); vitamins (for example, vitamins A, B complex, C, D, E, F, G, J, K, N, P, PP, T, U and their subspecies); amino acids such as arginine, histidine, proline, lysine, methionine, alanine, phenylalanine, aspartic acid, glutamic acid, glutamine, threonine, tryptophan, glycine, isoleucine, leucine, valine; prostaglandins (for example, E₁, E₂, F_{2α}, I₂); enzymes such as pepsin, pancreatin, rennin, papain, trypsin, pancrelipase, chymopapain, bromelain, chymotrypsin, streptokinase, urokinase, tissue plasminogen activator, fibrinolysin, desoxyribonuclease, sutilains, collagenase, asparaginase, heparinase; buffers and salts (for example, NaCl, cations including: Na⁺, K⁺, Ca⁺⁺, Mg⁺⁺, Zn⁺⁺, NH₄⁺ triethanolamine, anions including: phosphate, sulfate, chloride, citrate, ascorbate, acetate, borate, carbonate ions); preservatives (for example, benzalkonium chloride, Na or K bisulfite, Na or K thiosulfate, parabans); antigout agents (for example, allopurinol, cochicine, probenicid, sulfinpyrazone); antidepressant agents such as amitriptyline, amoxapine, desipramine, doxepin, imipramine, nortriptyline, protriptyline, trimipramine; contraceptives (for example, norethindrone combinations, such as with ethinyl estradiol or with mestranol); and antinauseants/antiemetic agents (for example, dimenhydrinate, hydroxyzine, meclizine, metoclopramide, prochlorperazine, promethazine, scopolamine, thiethylperazine, triethobenzamide).

Other therapeutic agents that can likewise be utilized within the present invention include antiasthmatic agents, antipsychotic agents, bronchodilators, gold compounds, hypoglycemic agents, hypolipedemic agents, anesthetics, vaccines, agents which affect bone metabolism, anti-diarrhetics, fertility agents, muscle relaxants, appetite suppressants, hormones such as thyroid hormone, estrogen, progesterone, cortisone and/or growth hormone, other biologically active molecules such as insulin, as well as T_{H1} (for example, interleukins (IL) IL-2, IL-12, and IL-15, interferon-γ) cytokines or T_{H2} (for example, IL-4 and IL-10) cytokines.

Although the above therapeutic agents have been provided for the purposes of illustration, it should be understood that the present invention is not so limited. For example, although agents are specifically referred to above, the present invention should be understood to include analogues, derivatives and conjugates of such agents. As an illustration, paclitaxel should be understood to refer to not only the common chemically available form of paclitaxel, but analogues (for example, taxotere, as noted above) and paclitaxel conjugates (for example, paclitaxel-PEG, paclitaxel-dextran, or paclitaxel-xylos). In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

### Fluorescent Dyes

Fluorescent dyes are well-known in the art and include indocyanin green (ICG), Evans Blue (EB), 4′6-diamidino-2-phenylindole (DAPI), lucifer yellow (LY), green fluorescent protein (GFP), red fluorescent protein (RFP) fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane, or analogs thereof).

### Polymeric Carriers

As noted above, therapeutic compositions of the present invention may additionally comprise a polymeric carrier. A wide variety of polymeric carriers may be utilized to contain and or deliver one or more of the therapeutic agents discussed above, including for example both biodegradable and non-biodegradable compositions. Representative examples of biodegradable compositions include albumin, collagen, gelatin, starch, cellulose (methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids and their copolymers (see generally Illum, L., Davids, S. S. (eds.) “Polymers in controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include EVA copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurathane, poly(ester urathanes), poly(ether urathanes), poly(ester-urea), polyethers (poly(ethylene oxide), poly(propylene oxide), pluronics, poly(tetramethylene glycol))xxx, silicone rubbers and vinyl polymers [polyvinylpyrrolidone, poly(vinyl alcohol, poly(vinyl acetate phthalate. Polymers may also be developed which are either anionic (for example, alginate, carrageenin, carboxymethyl cellulose and poly(acrylic acid), or cationic (for example, Chitosan, poly-1-lysine, polyethylenimine, and poly(allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int. J. Pharm. 118:257-263, 1995). Particularly preferred polymeric carriers include poly(ethylene-vinyl acetate) (40% cross-linked), poly(D,L-lactic acid) oligomers and polymers, poly(L-lactic acid) oligomers and polymers, poly(glycolic acid), copolymers of lactic acid and glycolic acid, poly(caprolactone), poly(valerolactone), polyanhydrides, copolymers of poly(caprolactone) or poly(lactic acid) with polyethylene glycol and blends thereof.

Polymeric carriers can be fashioned in a variety of forms, with desired release characteristics and/or with specific desired properties. For example, polymeric carriers may be fashioned to release a therapeutic agent upon exposure to a specific triggering event such as pH (see, for example, Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354, 1993; Dong et al., J. Controlled Release 19:171-178, 1992; Dong and Hoffman, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10):1544-1547, 1993; Serres et al., Pharm. Res. 13(2):196-201, 1996; Peppas, “Fundamentals of pH- and Temperature-Sensitive Delivery Systems,” in Gumy et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers 1, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid)), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water soluble polymer.

Likewise, polymeric carriers can be fashioned which are temperature sensitive (see, for example, Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels Poly N-isopropylacrylamide-co-sodium acrylate-co-n-N-alkylacrylamide Network Synthesis and Physicochemical Characterization,” Dept. of Chemical & Bioligal Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102, 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffmnan, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).

Representative examples of thermogelling polymers, and their gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N,n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water soluble polymers such as acrylmonomers (for example, acrylic acid and derivatives thereof such as methylacrylic acid, acrylate and derivatives thereof such as butyl methacrylate, acrylamide, and N-n-butyl acrylamide).

Other representative examples of thermogelling polymers include cellulose ether derivatives such as hydroxypropyl cellulose, 41° C.; methyl cellulose, 55° C.; hydroxypropylmethyl cellulose, 66°.; and ethylhydroxyethyl cellulose, and pluronics such as F-127, 10-15° C.; L-122, 19° C.; L-92, 26° C.; L-81, 20° C.; and L-61, 24° C.

The polymer can be in a number of phases, such as, but not limited to, a liquid phase, a gel phase, a solid phase, or any combination thereof. The therapeutic composition of the present invention can be in a number of phases, such as, but not limited to, a liquid phase, a gel phase, a solid phase, or any combination thereof. Preferably, the therapeutic compositions are fashioned in a manner appropriate to the intended use. Within certain aspects of the present invention, the therapeutic composition should be biocompatible, and release one or more therapeutic agents or drugs over a period of several days to months. For example, “quick release” or “burst” therapeutic compositions are provided that release greater than 10%, 20%, or 25% (w/v) of a therapeutic agent (for example, thalidomide or neomycin) over a period of 7 to 10 days. Such “quick release” compositions should, within certain embodiments, be capable of releasing chemotherapeutic levels (where applicable) of a desired agent. Within other embodiments, “low release” therapeutic compositions are provided that release less than 1% (w/v) of a therapeutic agent over a period of 7 to 10 days. Further, therapeutic compositions of the present invention should preferably be stable for several months and capable of being produced and maintained under sterile conditions.

### Formulation and Medical Device Coating Procedure

Representative examples of the incorporation of therapeutic agents into a polymeric carrier and coating or binding to the medical device such as stent or polymer tube are described in more detail below in the Examples section. The therapeutic agents can be incorporated with the polymeric carrier at a ratio (w/w) of, for example, about 1:99, 2:98, 5:95, 10:90, 15:85, 20:80, 25:75, 30:70, 33:67, 35:65, 40:60, 45:55, 50:50, and any inverse ratio thereof.

### Composition of the Stent

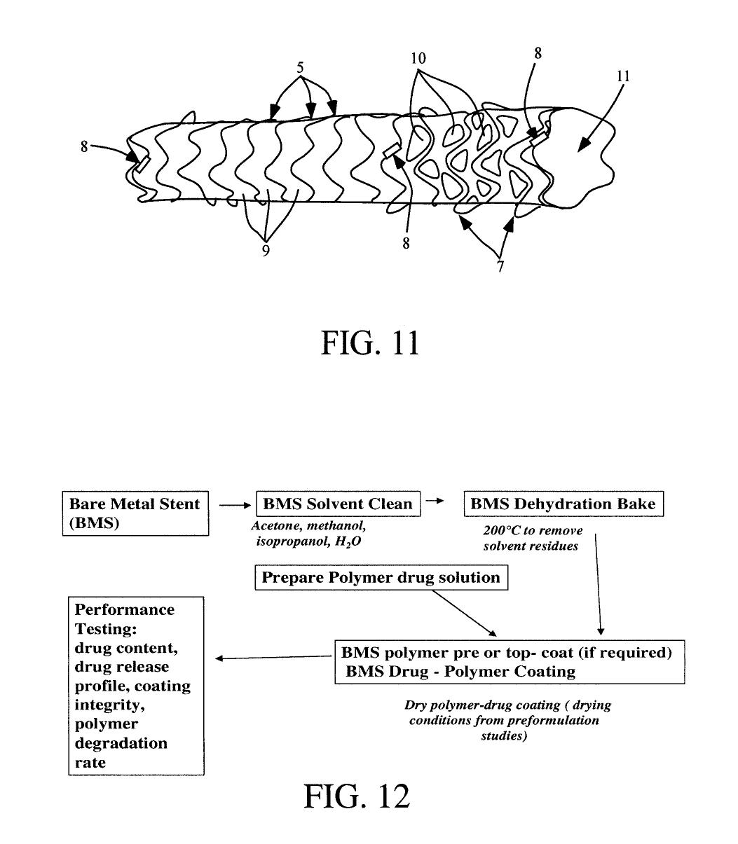

The structural and support elements of the invention, for example, stents, tubes, catheters, channels, fibers, platforms, sheaths, rings, coils, needles, or the like, disclosed herein can be constructed from a variety of materials including polymers such as silicone, polyurethane, polyethylene, acrylonitrile butadiene styrene (ABS), polycarbonate, polypropylene, styrene, polyamide (nylon), polyimide, PEEK, PEBAX, polyester, PVC, fluoropolymers (TEFLON), and co-polymers thereof. Reinforcement elements such as metallic (stainless steel, nickel-titanium alloy (for example, NITINOL), and chromel) or polymeric braids or coils can be used in construction. The support can be a stent, such as, but not limited to, a stent graft, a self expanding stent, a tracheal and bronchial stent (including bilateral stem stents), a biliary stent, a temporary removable stent and/or stent graft, such as disclosed by Petersen et al. (2000, J. Vasc. Interv. Radiol. 11: 919-929), a covered Gianturco Z Stent, such as disclosed by Miyayama et al. (1997, J. Vasc. Interv. Radiol. 8: 641-648), a polyurethane covered Wallstent, such as disclosed by Rossi et al. (1997, Cardiovasc. Intervent. Radiol. 20: 441-447), a silicone stent and a drug-eluting stent, such as disclosed on FIG. 11. The drug-eluting stent can be bio-degradable or non-bio-degradable, or a composite having both properties. The drug-eluting stent can be a stent graft. Metal and other conductive materials can be used to conduct electrical current along the length of the stent. These conductive elements could be constructed of stainless steel, copper, gold, platinum, silver, titanium, NITINOL, conductive epoxy, and conductive polymers. Elements could be included in construction to make the stent more visible to x-ray imaging. These elements can include tantalum, platinum, iridium, gold, stainless steel, silver, nickel-titanium alloys, and polymer compounding agents such as barium sulfate and titanium oxide.

Exemplary stents that may be used with the invention include, but are not limited to, a tracheal and bronchial stent, such as NOVATECH DUMON (Boston Medical Products, Westborough, Mass.), a biliary stent, such as WALLSTENT (Meditech, Natick, Mass.), MESOTHERM (C.R. Bard, Inc, Billerica, Mass.), ZILVER STENT (Cook, Bloomington, Ind.), SMARTSTENT (Cordis, Miami, Fla.) and a flexible stent, such as ULTRAFLEX NITINOL stent (Boston Scientific, Minneapolis, Minn.).

### Composition of Coating for Medical Device or Stent

The embodiments of the composition for a primer layer are prepared by conventional methods wherein all components are combined, then blended. More particularly, in accordance to one embodiment, a predetermined amount of a polymer or a prepolymer is added to a predetermined amount of a solvent or a combination of solvents. The mixture can be prepared in ambient pressure and under anhydrous atmosphere. If necessary, a free radical or UV initiator can be added to the composition for initiating the curing or cross-linking of the prepolymer. Heating and stirring and/or mixing can be employed to effect dissolution of the polymer into the solvent.

Biocompatible polymers are to be used for the primer material. Examples of biocompatible primers include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonates), poly(iminocarbonate), copoly(ether-esters) (for example PEO/PLA), polyalkylene oxalates, polyphosphazenes and biomolecules such as fibrin, fibrinogen, cellulose, starch, collagen and hyaluronic acid. Also, polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The solvent or wetting fluids should be mutually compatible with the polymer and should be capable of placing the polymer into solution at the concentration desired in the solution. Useful solvents should also be able to expand the chains of the polymer for maximum interaction with the surface of the device, such as a metallic surface of a stent. Examples of solvent and wetting fluid can include, but are not limited to, dimethylsulfoxide (DMSO), chloroform, water (buffered saline), xylene, acetone, methanol, ethanol, 1-propanol, tetrahydrofuran, 1-butanone, dimethylformamide, dimethylacetamide, cyclohexanone, ethyl acetate, methylethylketone, propylene glycol monomethylether, isopropanol, N-methylpyrrolidinone, toluene, tetrahydrofuran (THF), dimethylformamide (DMF), 1-butanol, n-butyl acetate, dimethyl acetamide (DMAC), and mixtures and combinations thereof, and mixtures thereof. The solvent or wetting fluid should be mutually compatible with the polymer and the solvent and should not precipitate the polymer.

### Methods for Applying the Compositions to the Device

An exemplary process for coating a device or a stent with the compositions of the invention is illustrated on FIG. 12. To form the primer layer, the surface of the device or prosthesis should be clean and free from contaminants that may be introduced during manufacturing. However, the surface of the prosthesis requires no particular surface treatment to retain the applied coating. Metallic surfaces of stents can be, for example, cleaned by argon plasma process as is well known to one of ordinary skill in the art. Application of the composition can be by any conventional method, such as by spraying the composition onto the prosthesis or immersing the prosthesis in the composition. Operations such as wiping, centrifugation, blowing, or other web clearing acts can also be performed to achieve a more uniform coating. Briefly, wiping refers to physical removal of excess coating from the surface of the stent; centrifugation refers to rapid rotation of the stent about an axis of rotation; and blowing refers to application of air at a selected pressure to the deposited coating. The excess coating can also be vacuumed off the surface of the device. The addition of a wetting fluid leads to a consistent application of the composition, which also causes the coating to be uniformly deposited on the surface of the prosthesis.

With the use of the thermoplastic polymers, such as ethylene vinyl alcohol copolymer, polycaprolactone, poly(lactide-co-glycolide), poly(hydroxybutyrate), etc., the deposited primer composition should be exposed to a heat treatment at a temperature range greater than about the glass transition temperature and less than about the melting temperature of the selected polymer.

Unexpected results have been discovered with treatment of the composition under this temperature range, specifically strong adhesion or bonding of the coating to the metallic surface of a stent. The device should be exposed to the heat treatment for any suitable duration of time, which would allow for the formation of the primer coating on the surface of the device and allows for the evaporation of the solvent or combination of solvent and wetting fluid. It is understood that essentially all of the solvent and the wetting fluid will be removed from the composition but traces or residues can remain blended with the polymer.

### Composition for Forming the Therapeutic Agent or Active Ingredient Layer

The embodiments of the composition for an active ingredient-containing or reservoir layer are prepared by conventional methods wherein all components are combined, then blended. More particularly, a predetermined amount of a polymeric compound is added to a predetermined amount of a mutually compatible solvent or combination of solvents. The polymeric compound can be added at ambient pressure and under anhydrous atmosphere. If necessary, gentle heating and stirring and/or mixing can be employed to effect dissolution of the polymer into the solvent, for example 12 hours in a water bath at about 60.degree. C. The polymer chosen must be a polymer that is biocompatible and minimizes irritation to the vessel wall when the device is implanted. The polymer may be either a biostable or a bioabsorbable polymer. Bioabsorbable polymers that could be used include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (for example PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The choice of polymer for the reservoir layer can be the same as or different from the selected polymer for the primer layer. The use of the same polymer significantly reduces or eliminates any interfacial incompatibilities, such as lack of an adhesive tie or bond, which may exist with the employment of two different polymeric layers. In effect, it can be said that the use of the same polymeric material for the primer layer and the reservoir layer results in the formation of a single-layered coating.

The solvent should be capable of placing the polymer into solution at the concentration desired in the solution. Examples of solvent can include, but are not limited to, DMSO, chloroform, acetone, water (buffered saline), xylene, methanol, ethanol, 1-propanol, tetrahydrofuran, 1-butanone, dimethylformamide, dimethylacetamide, cyclohexanone, and N-methylpyrrolidinone. With the use of low ethylene content, for example, 29 mol %, ethylene vinyl alcohol copolymer, a suitable choice of solvent is iso-propylalcohol admixed with water.

Sufficient amounts of an active ingredient are dispersed in the blended composition of the polymer and the solvent. The active ingredient or therapeutic agent should be in true solution in the blended composition. The preferred active ingredient is neomycin, thalidomide, derivatives or analogues thereof. The concentration of the active ingredient required to produce a favorable therapeutic effect should be less than the level at which the active ingredient produces toxic effects and greater than the level at which non-therapeutic results are obtained.

By way of example, the polymer can comprise from about 0.1% to about 35% by weight of the total weight of the composition, the solvent can comprise from about 59.9% to about 99.8 by weight of the total weight of the composition, and the active ingredient can comprise from about 0.1% to about 99.9%, by weight of the total weight of the composition.

### Composition for Forming the Release Rate Reducing Membrane

The embodiments of the composition for a rate-reducing membrane or diffusion barrier layer are prepared by conventional methods wherein all components are combined. In the embodiment with the use of particles, dispersion techniques should also be employed to circumvent agglomeration or formation of particle flocs.

More particularly, the embodiments for the composition for the reservoir layer can be applied on a selected region of the reservoir layer to form a rate reducing member or a barrier layer. The barrier layer can reduce the rate of release or delay the time at which the active ingredient is released from the reservoir layer. In one embodiment, for maximum blood compatibility, polyethylene glycol or polyethylene oxide can also be added to the blend. Ethylene vinyl alcohol is functionally a very suitable choice of polymer. The copolymer allows for good control capabilities over the release rate of the active ingredient. Usefully, the choice of polymer for the barrier layer can be the same as the selected polymer for the reservoir. The use of the same polymer, as described for some of the embodiments, significantly reduces or eliminates any interfacial incompatibilities, such as lack of adhesion, which may exist in the employment of two different polymeric layers. In effect, it can be said that the use, if desired, of the same polymeric material for the barrier layer and the reservoir layer results in the formation of a single-layered coating. In other words, the use of the same polymeric material results in a seamless multi-layered coating in which the layers vary in terms of their content. Defined interfacial boundaries are, accordingly, significantly reduced or eliminated.

Exemplary active ingredients are those medicinal agents wherein gastric release is preferred over intestinal release or wherein control of the rate of release of the active agent is desired for systemic action. For example, drugs in which delivery to the stomach is preferred include natural or synthetic prostaglandins and prostaglandin analogues and prostacyclins, (e.g., misoprostol, enisoprost, enprostil, iloprost, and arbaprostil) any drugs for the treatment of peptic ulcers, gastric antisecretory drugs, antimicrobial drugs, prokinetic drugs, cytoprotective drugs and the like. Exemplary antimicrobial drugs include tetracycline, metronidazole and erythromycin which can be used for eradication of gastric microbes such as Heliobacter pylori.

The formulations may be administered alone or in combination with at least one other agent, such as a stabilizing compound, which may be administered in any sterile, biocompatible pharmaceutical carrier including, but not limited to, saline, buffered saline, dextrose, and water. The formulations may be administered to a patient alone, or in combination with other agents, drugs, or hormones.

In addition to the active ingredients, these pharmaceutical formulations may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Mack Publishing Co., Easton, Pa.).

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

The pharmaceutical formulations of the present invention may be manufactured in a manner that is known in the art, for example, by means of conventional mixing, dissolving, granulating, dragee-making, levigating, emulsifying, encapsulating, entrapping, or lyophilizing processes. The pharmaceutical formulation may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms. In other cases, the preferred preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

Pharmaceutical formulations suitable for use in the invention include formulations wherein the active ingredients are contained in an effective amount to achieve the intended purpose. The determination of an effective dose is well within the capability of those skilled in the art.

For any compound, the therapeutically effective dose can be estimated initially either in cell culture assays, for example, of neoplastic cells or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, and it can be expressed as the LD₅₀/ED₅₀ ratio. Pharmaceutical formulations which exhibit large therapeutic indices are preferred. The data obtained from cell culture assays and animal studies are used to formulate a range of dosage for human use. The dosage contained in such formulations is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical formulations may be administered every 3 to 4 days, every week, or biweekly depending on the half-life and clearance rate of the particular formulation.

Normal dosage amounts may vary from about 0.1 μg to 100,000 μg, up to a total dose of about 1 g, depending upon the route of administration. Guidance as to particular dosages and methods of delivery is provided in the literature and generally available to practitioners in the art.

In some embodiments, the formulation contains at least 1% by weight of the drug. For example, the formulation can contain at least 1%, at least 2%, at least 5%, at least 7%, at least 10%, at least 15%, at least 17%, at least 20%, at least 30%, at least 40%, at least 45% at least 50%, at least 60%, or at least 70%, e.g. 1-20%, 5-30%, 10-30%, 10-50%, 20-30% or 20-50% by weight of the drug. In other embodiments, the formulation can contain less than 1% of the drug.

### The Support

The support, device, or prosthesis used in conjunction with the above-described compositions may be any suitable device used for the release of an active ingredient, examples of which include bio-erodible stents, polymer tubes, self-expandable stents, balloon-expandable stents, and stent-grafts, and grafts. In the alternative, the above-described compositions can be used in conjunction with another polymer having similar properties, the polymer previously shaped and formed for use in a body passageway. In another alternative, the above-described compositions can be used in conjunction with another polymer having different properties, the polymer previously shaped and formed for use in a body passageway.

The stent can also comprise an optional marker, the marker having use as an aid for locating and/or monitoring the position of the stent within the body passageway during and/or after a surgical procedure. The marker can comprise a radiopaque composition, such as a radiopaque dye, a radio-opaque material, a magnet, echogenic material, an ion source, such as a radio-isotope, or the like.

### Methods for Applying the Compositions to the Device

To form the primer layer the surface of the device or prosthesis should be clean and free from contaminants that may be introduced during manufacturing. However, the surface of the prosthesis requires no particular surface treatment to retain the applied coating. Metallic surfaces of stents can be, for example, cleaned by argon plasma process as is well known to one of ordinary skill in the art. Application of the composition can be by any conventional method, such as by spraying the composition onto the prosthesis or immersing the prosthesis in the composition. Operations such as wiping, centrifugation, blowing, or other web clearing acts can also be performed to achieve a more uniform coating. Briefly, wiping refers to physical removal of excess coating from the surface of the stent; centrifugation refers to rapid rotation of the stent about an axis of rotation; and blowing refers to application of air at a selected pressure to the deposited coating. The excess coating can also be vacuumed off the surface of the device. The addition of a wetting fluid leads to a consistent application of the composition, which also causes the coating to be uniformly deposited on the surface of the prosthesis.

With the use of the thermoplastic polymers, such as ethylene vinyl alcohol copolymer, polycaprolactone, poly(lactide-co-glycolide), poly(hydroxybutyrate), etc., the deposited primer composition should be exposed to a heat treatment at a temperature range greater than about the glass transition temperature (T_{g}) and less than about the melting temperature (Tₘ) of the selected polymer. Unexpected results have been discovered with treatment of the composition under this temperature range, specifically strong adhesion or bonding of the coating to the metallic surface of a stent. The device should be exposed to the heat treatment for any suitable duration of time, which would allow for the formation of the primer coating on the surface of the device and allows for the evaporation of the solvent or combination of solvent and wetting fluid. It is understood that essentially all of the solvent and the wetting fluid will be removed from the composition but traces or residues can remain blended with the polymer.

As discussed in more detail below, therapeutic agents of the present invention, which are optionally incorporated within one of the carriers described herein to form a therapeutic composition, may be prepared and utilized to treat or prevent a wide variety of diseases.

### Treatment or Prevention of Disease

As noted above, the present invention provides methods for treating or preventing a wide variety of diseases associated with the obstruction of body passageways, including for example, vascular diseases, neoplastic obstructions, inflammatory diseases, and infectious diseases.

In one aspect, the invention is used to treat or prevent an obstruction of a body passageway, wherein the body passageway is a coronary artery, a carotid artery, an aorta, a pulmonary artery, an artery, a vein, a capillary, a trachea, a bronchus, bronchioles, an oesaphagus, a bile duct, fallopian tubes, a urethra, a colon, a bladder, a pancreatic passageway, a nasal passageways, a male reproductive tract, a female reproductive tract, a small intestine, a large intestine, a cranial sinus, and a brain sinus.

The invention may be used to treat, for example, malignant tracheal obstruction. FIG. 14 illustrates an example of different types of tracheal obstruction that may be treated using the invention. The invention mat be used as a multi-modal adjunct intervention device to treat airway obstruction in lung cancer due to endobronchial, extrinsic, and/or mixed tracheobronchial tumors. Using multiple processes, such as, but not limited to, physical, chemical, biological, and molecular mechanisms, in conjunction with, for example, laser or electrosurgery, the invention may result in superior results and prognoses compared with a single endobronchial clinical intervention using an uncoated stent that can result in tissue hyperplasia and/or tumor in-growth.

For example, within one aspect of the present invention a wide variety of therapeutic compositions as described herein may be utilized to treat vascular diseases that cause obstruction of the vascular system. Representative examples of such diseases include artherosclerosis of all vessels (around any artery, vein or graft) including, but not restricted to: the coronary arteries, aorta, iliac arteries, carotid arteries, common femoral arteries, superficial femoral arteries, popliteal arteries, and at the site of graft anastomosis; vasospasms (for example, coronary vasospasms and Raynaud's Disease); restenosis (obstruction of a vessel at the site of a previous intervention such as balloon angioplasty, bypass surgery, stent insertion and graft insertion); inflammatory and autoimmune conditions (for example, Temporal Arteritis, vasculitis).

Briefly, in vascular diseases such as atherosclerosis, white cells, specifically monocytes and T lymphocytes adhere to endothelial cells, especially at locations of arterial branching. After adhering to the endothelium, leukocytes migrate across the endothelial cell lining in response to chemostatic stimuli, and accumulate in the intima of the arterial wall, along with smooth muscle cells. This initial lesion of athersosclerosis development is known as the “fatty streak”. Monocytes within the fatty streak differentiate into macrophages; and the macrophages and smooth muscle cells progressively take up lipids and lipoprotein to become foam cells.

As macrophages accumulate, the overlying endothelium becomes mechanically disrupted and chemically altered by oxidized lipid, oxygen-derived free radicals and proteases which are released by macrophages. Foam cells erode through the endothelial surface causing micro-ulcerations of the vascular wall. Exposure of potentially thrombogenic subendothelial tissues (such as collagen and other proteins) to components of the bloodstream results in adherence of platelets to regions of disrupted endothelium. Platelet adherence and other events triggers the elaboration and release of growth factors into this mileau, including platelet-derived growth factor (PDGF), platelet activating factor (PAF), and interleukins 1 and 6 (IL-1, IL-6). These paracrine factors are thought to stimulate vascular smooth muscle cell (VSMC) migration and proliferation.

In the normal (non-diseased) blood vessel wall, vascular smooth muscle cells have a contractile phenotype and low index of mitotic activity. However, under the influence of cytokines and growth factors released by platelets, macrophages and endothelial cells, VSMC undergo phenotypic alteration from mature contractile cells to immature secretory cells. The transformed VSMC proliferate in the media of the blood vessel wall, migrate into the intima, continue to proliferate in the intima and generate large quantities of extracellular matrix. This transforms the evolving vascular lesion into a fibrous plaque. The extracellular matrix elaborated by secretory VSMC includes collagen, elastin, glycoprotein and glycosaminoglycans, with collagen comprising the major extracellular matrix component of the atherosclerotic plaque. Elastin and glycosaminoglycans bind lipoproteins and also contribute to lesion growth. The fibrous plaque consists of a fibrous cap of dense connective tissue of varying thickness containing smooth muscle cells and overlying macrophages, T cells and extracellular material.

In addition to PDGF, IL-1 and IL-6, other mitogenic factors are produced by cells which infiltrate the vessel wall including: transforming growth factor β(TGF-β), fibroblast growth factor (FGF), thrombospondin, serotonin, thromboxane A₂, norepenephrine, and angiotension II. This results in the recruitment of more cells, elaboration of further extracellular matrix and the accumulation of additional lipid. This progressively enlarges the atherosclerotic lesion until it significantly encroaches upon the vascular lumen. Initially, obstructed blood flow through the vascular tube causes ischemia of the tissues distal to the atherosclerotic plaque only when increased flow is required—later as the lesion further blocks the artery, ischemia occurs at rest.

Macrophages in the enlarging atherosclerotic plaque release oxidized lipid, free radicals, elastases, and collageneses that cause cell injury and necrosis of neighbouring tissues. The lesion develops a necrotic core and is transformed into a complex plaque. Complex plaques are unstable lesions that can: break off causing embolization; local hemorrhage (secondary to rupture of the vasa vasora supplying the plaque which results in lumen obstruction due to rapid expansion of the lesion); or ulceration and fissure formation (this exposes the thrombogenic necrotic core to the blood stream producing local thrombosis or distal embolization). Even should none of the above sequela occur, the adherent thrombus may become organized and incorporated into the plaque, thereby accelerating its growth. Furthermore, as the local concentrations of fibrinogen and thrombin increase, proliferation of vascular smooth muscle cells within the media and intima is stimulated; a process which also ultimately leads to additional narrowing of the vessel.

The intima and media of normal arteries are oxygenated and supplied with nutrition from the lumen of the artery or from the vasa vasorum in the adventitia. With the development of atherosclerotic plaque, microvessels arising from the adventitial vasa vasorum extend into the thickened intima and media. This vascular network becomes more extensive as the plaque worsens and diminishes with plaque regression.

Hemorrhage from these microvessels may precipitate sudden expansion and rupture of plaque in association with arterial dissection, ulceration, or thrombosis. It has also been postulated that the leakage of plasma proteins from these microvessels may attract inflammatory infiltrates into the region and these inflammatory cells may contribute to the rapid growth of atherosclerotic plaque and to associated complications (through local edema and inflammation).

In order to treat vascular diseases, such as those discussed above, a wide variety of therapeutic agents (either with or without a carrier) may be delivered to the passageway via a medical device implant. Particularly preferred therapeutic agents in this regard include anti-angiogenic factors, inhibitors of platelet adhesion/aggregation (for example, aspirin, dipyridamole, thromboxane synthesis inhibitors, fish oils that result in production of thromboxane AE rather than the more potent thromboxane A2, antibodies against the platelet IIb/IIIa receptors that binds fibrinogen and prostacyclin), vasodilators (for example, calcium entry blockers, such as verapamil, and the nitric oxide donors nitroglycerine, nitroprusside, and molsidomine) and anthithrombotics and thrombin antagonists (for example, heparin (low-molecular-weight heparins, warfarin andudin). Other therapeutics which may be utilized include anti-inflammatory agents (for example, glucorticoids, dexamethasone and methylprednisolone), growth factor inhibitors (for example, PDGF antagonist such as trapidil; receptor inhibitors (for example, inhibitors of the receptors for FGF, VEGF, PDGF and TNF), including inhibitors of tyrosine kinase and promoters of tyrosine phosphatase; somatostatin analogs, including angiopeptin; angiotensin converting enzyme inhibitors; and 5HT₂ serotenergic receptor antagonists such as ketanserin). Yet other therapeutic agents include anti-proliferative agents (for example, colchicine, heparin, beta (for example, P-32) or gamma emitters (for example, Ir-192), calcium-entry blockers such as verapamil, diltiazem and nifedipine, cholesterol-lowering HMB Co-A reductase inhibitors such as lovastatin, compounds which disrupt microtubule function such as paclitaxel and nitric oxide donors as discussed above), and promoters of re-endothelialization (for example, bFGF and vascular endothelial cell growth factor).

Within other aspects of the invention, the therapeutic agents or compositions described herein may be utilized to treat neoplastic obstructions. Briefly, as utilized herein, a “neoplastic obstruction” should be understood to include any neoplastic (benign or malignant) obstruction of a bodily tube regardless of tube location or histological type of malignancy present. Representative examples include gastrointestinal diseases (for example, oral-pharyngeal carcinoma (adenocarcinoma), esophageal carcinoma (squamous cell, adenocarcinoma, lymphoma, melanoma), gastric carcinoma (adenocarcinoma, linitis plastica, lymphoma, leiomyosarcoma), small bowel tumors (adenomas, leiomyomas, lipomas, adenocarcinomas, lymphomas, carcinoid tumors), colon cancer (adenocarcinoma) and anorectal cancer); biliary tract diseases (for example, neoplasms resulting in biliary obstruction such as pancreatic carcinoma (ductal adenocarcinoma, islet cell tumors, cystadenocarcinoma), cholangiocarcinoma and hepatocellular carcinoma); pulmonary diseases (for example, carcinoma of the lung and/or tracheal/bronchial passageways (small cell lung cancer, non-small cell lung cancer); female reproductive diseases (for example, malignancies of the fallopian tubes, uterine cancer, cervical cancer, vaginal cancer); male reproductive diseases (for example, testicular cancer, cancer of the epididymus, tumors of the vas deferens, prostatic cancer, benign prostatic hypertrophy); and urinary tract diseases (for example, renal cell carcinoma, tumors of the renal pelvis, tumors of the urinary collection system such as transitional cell carcinoma, bladder carcinoma, and urethral obstructions due to benign strictures, or malignancy).

As an example, benign prostatic hyperplasia (BPH) is the enlargement of the prostate, particularly the central portion of the gland which surrounds the urethra, which occurs in response to prolonged androgenic stimulation. It affects more than 80% of the men over 50 years of age. This enlargement can result in compression of the portion of the urethra which runs through the prostate, resulting in bladder outflow tract obstruction, i.e., an abnormally high bladder pressure is required to generate urinary flow. In 1980, 367,000 transurethral resections of the prostate were performed in the United States as treatment for BPH. Other treatments include medication, transurethral sphincterotomy, transurethral laser or microwave, transurethral hyperthermia, transurethral ultrasound, transrectal microwave, transrectal hyperthermia, transrectal ultrasound and surgical removal. All have disadvantages including interruption of the sphincter mechanism resulting in incontinence and stricture formation.

In order to treat neoplastic diseases, such as those discussed above, a wide variety of therapeutic agents (either with or without a polymeric carrier) may be delivered to the body passageway. Particularly preferred therapeutic agents in this regard include anti-angiogenic, anti-proliferative or anti-neoplastic agents discussed above, including for example, compounds such as paclitaxel and derivatives or analogues thereof, or neomycine and derivatives or analogues thereof.

Within other aspects of the invention, methods are provided for preventing or treating inflammatory diseases which affect or cause the obstruction of a body passageway Inflammatory diseases include both acute and chronic inflammation which result in obstruction of a variety of body tubes. Representative examples include vasculitis (for example, Giant cell arteritis (temporal arteritis, Takayasu's arteritis), polyarteritis nodosa, allergic angiitis and granulomatosis (Churg-Strauss disease), polyangiitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system), Wegener's granulomatosis, Kawasaki's disease, vasculitis of the central nervous system, Buerger's disease and systemic sclerosis); gastrointestinal tract diseases (for example, pancreatitis, Crohn's Disease, Ulcerative Colitis, Ulcerative Proctitis, Primary Sclerosing Cholangitis, benign strictures of any cause including ideopathic (for example, strictures of bile ducts, esophagus, duodenum, small bowel or colon)); respiratory tract diseases (e.g, asthma, hypersensitivity pneumonitis, asbestosis, silicosis, and other forms of pneumoconiosis, chronic bronchitis and chronic obstructive airway disease); nasolacrimal duct diseases (for example, strictures of all causes including ideopathic); and eustachean tube diseases (for example, strictures of all causes including ideopathic).

In order to treat inflammatory diseases, such as those discussed above, a wide variety of therapeutic agents may be delivered to the body passageway, or to smooth muscle cells via a medical device implant. Particularly preferred therapeutic agents in this regard include both nonsteroidal agents (“NSAIDS”) and steroidal agents, as well as the anti-angiogenic factors discussed above. Other agents which may also be utilized include a wide variety of anti-angiogenic facts such as thalidomide and neomycin.

Within yet other aspects of the present invention, methods are provided for treating or preventing infectious diseases that are associated with, or causative of, the obstruction of a body passageway. Briefly, infectious diseases include several acute and chronic infectious processes can result in obstruction of body passageways including for example, obstructions of the male reproductive tract (for example, strictures due to urethritis, epididymitis, prostatitis); obstructions of the female reproductive tract (for example, vaginitis, cervicitis, pelvic inflammatory disease (for example, tuberculosis, gonococcus, chlamydia, enterococcus and syphilis); urinary tract obstructions (for example, cystitis, urethritis); respiratory tract obstructions (for example, chronic bronchitis, tuberculosis, other mycobacterial infections (MAI, etc.), anaerobic infections, fungal infections and parasitic infections) and cardiovascular obstructions (for example, mycotic aneurysms and infective endocarditis).

In order to treat infectious diseases, such as those discussed above, a wide variety of therapeutic agents (either with or without a carrier) may be delivered to the body passageway, or to smooth muscle cells via a medical device implant. Particularly preferred therapeutic agents in this regard include neomycin and a wide variety of antibiotics as discussed above.

### Selection of Drug

Paclitaxel (PTX) is an active ingredient in a drug-eluting tracheo-bronchial stent as it meets many of the criteria required for local drug delivery. PTX exhibits broad spectrum anti-tumor activity, has demonstrated therapeutic potential in lung cancer patients and has minimal pulmonary toxicity. There is pre-clinical evidence to support therapeutic activity of PTX when applied loco-regionally to a lung tumor. (See, for example, Creel et.al. (2000) Circulation Res. 86: 879-884; Kuh et al. (1999) J. Pharmacol. Exp. Ther. 290: 871-880; see Aphios web site: world wide web at <aphios.com>, folder “pipeline”, and document “dermos.html”; Jackson et al. (2000) Cancer Res. 60: 4146-4151; Sousa et al. (2003) Circulation 107: 2274; Herdeg et al. (2000) J. Am. Coll. Cardiol. 35: 1969-1976; Gautam et.al. (2003) Curr. Cancer Drug Targets 3: 287-296).

Thalidomide (THM) has multiple modes of action. It is a potent immuno-modulatory, anti-angiogenic and anti-inflammatory drug (see Meierhofer et al. (2001) Biodrugs 15: 681-703; Puckmann et al. (2000) Drugs, 60: 273-292; Moreira et al. (1993) J. Exp. Med., 177: 1675-1680; Calabrese et al. (2000) Am. J. Med., 108: 487-495; Mujagic et.al. (2002) Croat. Med. J., 43: 274-285). It is non-cytotoxic and has a mechanism of action different from Paclitaxel.

Immunomodulation: Thalidomide is an immunomodulatory agent with broad spectrum of effects on immune function, cytokine secretion, angiogenesis, and cell adhesion and cell proliferation (Meierhofer et al. (2001) supra; Puckmann et al. (2000) supra; Moreira et al. (1993) supra; Calabrese et al. (2000) supra; and Mujagic et.al. (2002) supra) Immune modulation and anti-angiogenic properties are believed to be important for the anti-tumor activity of thalidomide and these in turn may be mediated through the drug's multiple actions on cellular cytokine secretion. The most pronounced effect of thalidomide is that on TNF-alpha generation and release, a cytokine that is involved in the up-regulation of endothelial cell integrin expression a process crucial for new blood vessel formation.

Anti-angiogenic activity: Thalidomide has a strong anti-angiogenic activity in vascular endothelial growth factor (VEGF)— and basic fibroblast growth (bFGF)-induced angiogenesis. These effects are especially important in the treatment of diseases involving neoformation of blood vessels including most malignancies.

Promising clinical trials: Thalidomide has shown promise in clinical trials in liver and lung cancer (see, for example, Dmato et al. (1994) Proc. Natl. Acad. Sci. 91: 4082-4085; Kong et al. (2001) Proc. Am. Soc. Clin. Oncol. 20: 133b; Patt et al. (2000) Proc. Am. Soc. Clin. Oncol. 14: 266a; Schwartz et al. (2002) Proc. Am. Soc. Clin. Oncol. 21:10b; Hsu et al. (2003) Oncology 65: 242-249; Wang et al. (2004) World J. Gastroenterol. 10: 649-653; Mise et al. (1996) Hepatology 23: 455-464; Hsu et al. (1997) Anticancer Res. 17: 2803-2809; and Poon et al. (2002). J. Clin. Oncol. 20: 1775-1785). Preliminary reports suggested response rates of 4%-10% and disease control rates of 8%-57% but have been associated with systemic toxicities related to thalidomide.

Pharmacokinetic advantage: Loco-regional delivery of thalidomide provides a pharmacokinetic advantage and enhance the therapeutic effectiveness by providing longer drug residence times and higher concentrations while minimizing systemic side affects of thalidomide (see, for example, Collins (1984) J. Clin. Oncol. 2: 498-504).

Physico-chemical Profile of Thalidomide: Thalidomide (C₁₃H₁₀N₂O₄) is phthalimidoglutarimide. One of a number of systematic names is 2-(2,6-dioxo-3-piperidinyl)-1H-isoindole-1,3(2H)-dione). Commonly available Thalidomide is racemic. The enantiomers are converted to each other in vivo.

Combination PTX and THM: Superiority of multi-drug therapy with complimentary mechanism of actions is standard therapy in cancer treatment and is known to increase the therapeutic window. The in vitro and in vivo performance of loco-regionally applied dual-drug formulations of PTX and THM (or any other pair of drugs) can be developed and evaluated.

### Profile of Self-Expanding Metallic Stent:

Self-expanding metallic stents (SEMS) are used to treat obstructed lumen in the tracheo-bronchial region. The Boston Scientific ULTRAFLEX stent is the current industry standard for SEMS tracheo-bronchial stents. The uncovered ULTRAFLEX can be used as the support for the drug-eluting tracheo-bronchial stent. The ULTRAFLEX has been show to be effective in the treatment of obstructed lumen but shows problems similar to all SEMS, primarily tumor ingrowth and granulation tissue formation.

### Profile of Biodegradable Polymers:

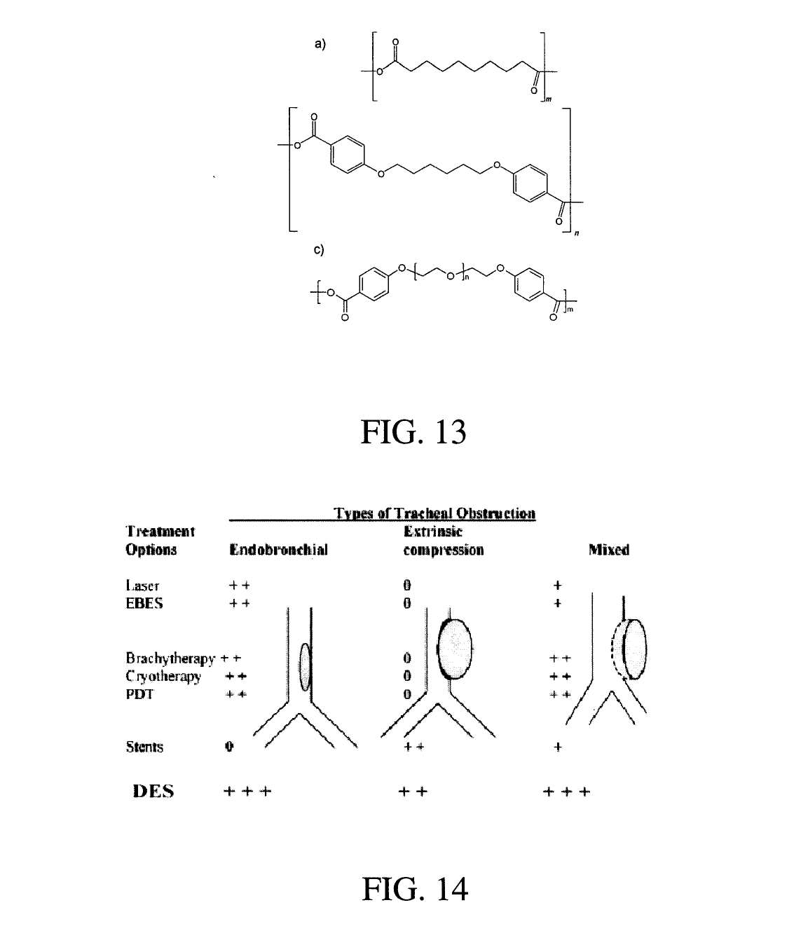

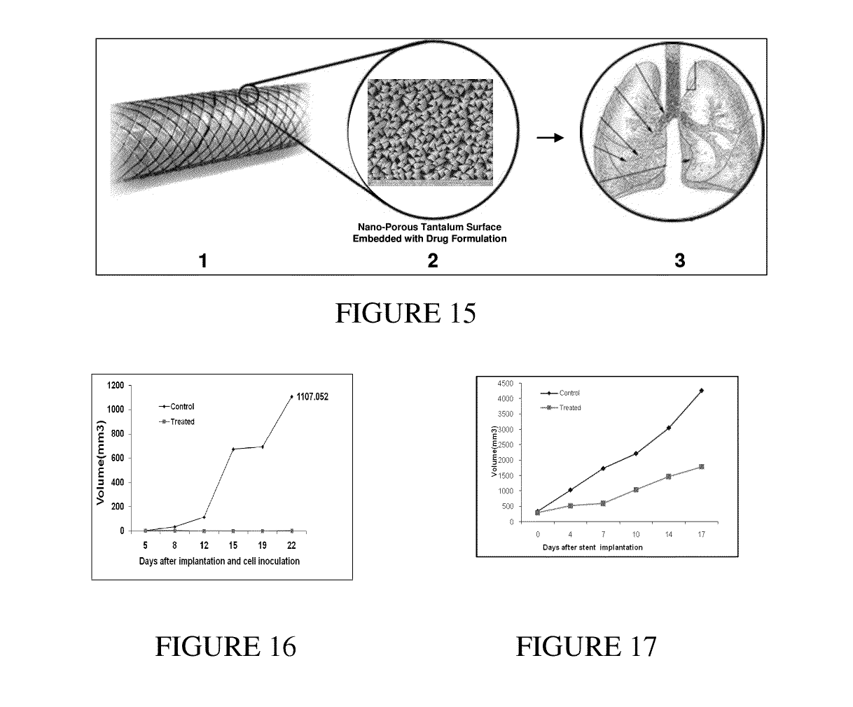

Surface-erodible polyanhydrides have been studied as potential drug delivery carriers for about two decades. Two polyanhydrides that can be used with the invention are disclosed in FIG. 13: co-monomers of sebacic acid (SA) (FIG. 13a) and of 1,6-bis(p-carboxyphenoxy) hexane) (CPH) (FIG. 13b). This class of water-insoluble polymer degrades into water-soluble monomers that can be absorbed by the body under conditions well known to those of skill in the art. By varying the ratio of the co-monomers, the degradation rate can be modified and/or adjusted from between about a few days to about a few months. In addition to the use of surface-erodible polyanhydride, poly CPH: SA copolymers used as a delivery vehicle a proprietary ampiphilic class of polyanhydride polymer can be used for evaluation as a delivery vehicle.

Polyanhydrides have been studied as potential drug delivery carriers for about two decades (Narasimhan and Kipper (2004) Chem. Engineer. 29: 169-218; Determan et al. (2004) J. of Control. Release 100: 97-109; Tamada and Langer (1992) J. Biomater. Sci. Polym. Ed. 3: 315-353; Ron, et al., (1993) Proc. Natl. Acad. Sci. 90: 4176-4180; Tabata et al., (1993) Pharm. Res. 10: 487-496). Polyanhydrides such as poly(carboxyphenoxy alkane-co-alkanoic acids), poly sebacic acid (SA) and 1,6-bis(p-carboxyphenoxy)hexane (CPH) (see FIGS. 413a and 413b) are a class of water-insoluble polymers that degrades (by backbone chain scission across the anhydride bond) into water-soluble monomers.

Narasimhan has designed a new class of ampiphilic polyanhydrides based on oligomeric ethylene glycol-containing anhydride monomers (for example, (1,8-bis(p-carboxyphenoxy)-3,6-dioxaoctane) (CPTEG) which are promising as novel drug carriers (Tones et al. (2006) J. Biomed. Mater. Res. 76: 102-110); and Vogel and Mallapragada (2005) J. Control Rel. 26: 721-728). These materials can be engineered to degrade much faster than hydrophobic polyanhydrides based on level of 1,6-bis(p-carboxyphenoxy)hexane) (CPH) in the polymer. The CPTEG-containing polyanhydrides degrade in a few days to a few weeks to a few months compared to SA and CPH containing polyanhydrides, which degrade over a few weeks to a few months. These polyanhydrides are copolymers based on poly(carboxyphenoxy alkane) and poly (CPTEG) (FIG. 13). The ampiphilic polyanhydride, poly(carboxyphenoxy alkane-co-CPTEG), degrade into water soluble monomers 1,6-bis-(carboxyphenoxy)hexane and oligomeric ethylene glycol. CPH monomers have been shown to be biocompatible in preclinical studies (Sampath and Brem (1998) Cancer Control Journal Vol. 3, Number 5 Supplemental; Leong et al. (1986) J. Biomed. Res. 20: 51-64; Harris (1992) In: Poly(ethylene glycol) Chemistry: Biotechnical and Biomed. Applications, Plenum Press, New York N.Y., pp 1-13; and Domb and Langer (1987) J. Polym. Sci., Polym. Chem. Ed. 25: 3373-3386) and oligomeric ethylene glycol is known for its biocompatibility and low toxicity (Harris (1992) supra).

### Pharmacology

Pharmaceutical compositions are those substances wherein the active ingredients are contained in an effective amount to achieve a desired and intended purpose. The determination of an effective dose is well within the capability of those skilled in the art.

For any compound, the therapeutically effective dose may be estimated initially either in cell culture assays or in animal models. The animal model is also used to achieve a desirable concentration range and route of administration. Such information may then be used to determine useful doses and routes for administration in humans. Pharmaceutically acceptable refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

A therapeutically effective dose refers to that amount of protein or inhibitor that ameliorates the symptoms or condition. Therapeutic efficacy and toxicity of such agents may be determined by standard pharmaceutical procedures in cell cultures or experimental animals, for example, ED₅₀ (the dose therapeutically effective in 50% of the population) and LD₅₀ (the dose lethal to 50% of the population). The dose ratio between toxic and therapeutic effects is the therapeutic index, and it may be expressed as the ratio, LD₅₀/ED₅₀. Pharmaceutical compositions that exhibit large therapeutic indexes are preferred. The data obtained from cell culture assays and animal studies are used in formulating a range of dosage for human use.

### Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

### Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Acute toxicity tests are based on a single administration of an agent to the subject to determine the symptomology or lethality of the agent. Mice and rats are most frequently used in these tests because their short reproductive cycle allows the production of the numbers of organisms needed to satisfy statistical requirements. Three experiments are conducted: (1) an initial dose-range-finding experiment, (2) an experiment to narrow the range of effective doses, and (3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, having a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

### Combinations of Components

The components of the invention, for example, the support, the polymer matrix, the drug, and/or the pharmaceutical formulation, can be combined in a variety of ways. FIGS. 1 through 10 illustrate examples of how the components may be combined in use. FIGS. 1, 3, 5, 7, and 9 illustrate the components forming part of a cylindrical or tubular structure. Corresponding FIGS. 2, 4, 6, 8, and 10 illustrate components combined together as layers, for example, on a flat substrate. FIGS. 5 and 6 illustrate a material that can be a mixture, in various ratios, of a support and a polymer matrix.

One of the key accomplishments of this work has been overcoming not only the stability issues related to ICG, a labile molecule with a short half life (t_{1/2}=<2 min) under physiological conditions and under acidic conditions (pH<4) but also contributing to the stability of HCFU at pH>4 especially physiological pH 7.4. ICG is not stable at acidic pH<4 and HCFU is not stable at pH>4. A suboptimal pH (4) for the dissomedia was chosen to preserve both and simultaneously quantitate them as they eluted. The ETDS product stabilized both ICG and HCFU for greater than 96 hours until they were eluted in the dissolution media. Highlights included stabilizing ICG and HCFU in the product, during the coating process, stabilizing them during the prolonged drug dissolution testing period and keeping them stabilized in vivo for a 2-4 week period as demonstrated in the theranositc whole body imaging study and in a PK study—following examples section—Examples XI to XVI). An optimal test method that retained the stability of all 3 analytes especially HCFU and ICG which are stable at different pH ranges was developed. The formulation and process stabilized the ICG and HCFU coated on the nanoporous product.

The technical feasibility of developing a multi-functional Esophageal Theranostic Delivery Stent (ETDS) product with a nanoporous theranostic coat, for esophageal cancer has been demonstrated.

Stabilization and release of combination therapeutics, Paclitaxel (PTX), Carmofur (HCFU) and the labile dye, Indocyanine Green (ICG) have been done at an engineered slow, medium and fast-release rate.

High-drug loads ranging from 50-100 mg have shown to be feasible.

Analytical and bioanalytical methods have been developed and qualified to test the performance of the product and analyze drug levels in biological samples.

Proof-of-Concept evidence of theranostic effectiveness in a mouse xenograft model has been obtained and an efficacious dose, 0.43 mg/Kg/day has been identified. This dose is less than 1/10^{th} of the systemic efficacious dose and is less toxic as seen from steady body weights. The efficacy without toxicity is likely related to sustained exposure of the drugs.

The fast release ETDS delivers efficacious drug over a 1 month-period. Correlating in vitro release profiles to the in vivo release rate, the medium and slow release ETDS are projected to deliver drugs over a 1-3 month period.

Compelling preclinical in-house data, identification of prototype ETDS formulations and an efficacious dose and formulations, support further development and a transition to SBIR Phase II. The immediate goal would be demonstrating POC safety in IDE-enabling large animal trials, manufacturing of GMP/GLP quality ETDS and human POC trials.

### REFERENCE NUMBERS IN FIGURES

1. Support
2. First Polymer Matrix
3. Drug
4. Second Polymer Matrix
5. Metal or Alloy
6. Pharmaceutical Formulation
7. Anchoring Fin
8. Marker
9. Lining
10. Drainage Aperture
11. Lumen of stent

The invention will be more readily understood by reference to the following examples, which are included merely for purposes of illustration of certain aspects and embodiments of the present invention and not as limitations.

### EXAMPLES

### Example I

### Synthesis and Characterization of Biodegradable Polyanhydrides

Melt polycondensation methods are used to synthesize polyanhydride copolymers based on the monomers, SA, CPH, and CTEG. The prepolymers are prepared by refluxing dicarboxylic acids with acetic anhydride and purified by recrystallization. The other dicarboxylic acid monomers, CPH and CPTEG are synthesized by previously described methods (Narasimhan, B., and M. J. Kipper 2004) supra; Tones et al. (2006) supra; and Vogel and Mallapragada (2005) supra) The EG containing diacids will be synthesized by end functionalizing halogenated tri-EG units with p-hydroxybenzoic acid (Narasimhan, B., and M. J. Kipper, 2004, supra; Determan et al. (2004) supra). The copolymer compositions are chosen so as to vary the degradation times from a few days to a few months. The chemical structure, thermal and mechanical properties, and the degradation rate of the polymers are characterized.

Preformulation screening studies: Optimal drug to polymer ratio is identified based upon physical and chemical solubility, stability and compatibility of the polymer and drug in organic solvents at different conditions simulating formulation and coating process.

### Formulation Trials and Coating Process Development:

When a stent is used as a support, loading of PTX, THM and PTX-THM is in the range of 0.05-50 mg/stent or greater; the range of controlled drug-release rates is from between 1% to 25% released in 24-48 hours and 100% released from 0.5-3 months; and the range of polymer biodegradation is from 1 to 3 months.

### Example II

### Synthesis and Characterization of Biodegradable Polymers

Polyanhydride copolymers based on SA, CPH, and CPTEG are synthesized by melt polycondensation at 180° C. under vacuum (<0.3 ton) from mixed acetylated prepolymers (Narasimhan, B., and M. J. Kipper. 2004) supra; Tones et al. (2006) supra; and Vogel and Mallapragada (2005) supra). The prepolymers are prepared by refluxing dicarboxylic acids with acetic anhydride and purified by recrystallization. SA is purchased from Sigma Aldrich (St. Louis, Mo.). The other dicarboxylic acid monomer, CPH and CPTEG are synthesized by previously described methods [1, (Torres et al. (2006) supra; and Vogel and Mallapragada (2005) supra)]. In addition to the homopolymers, copolymers of SA, CPH, and CPTEG are synthesized. The polymers are characterized by ¹H nuclear magnetic resonance (¹H NMR) and IR spectroscopy to verify the chemistry and purity, gel permeation chromatography (GPC) to determine the molecular weight, differential scanning calorimetry (DSC) to determine the thermal properties, and dynamic mechanical analysis (DMA) to determine the mechanical properties.

Preformulation screening: The solubility of PTX and THM in polymer solutions (0-50% w/v) in ethanol, acetone, dichoromethane, acetonitrile and dimethysulfoxide and dimethylacetamide is determined. The stability of solutions bracketing the highest and lowest ratio of drug to polymer ratios, where both the drug and the polymer are solubilized is evaluated at 5° C., 25° C./75% humidity and 40° C./60% humidity for 2 weeks.

### Example III

### Development of Analytical Methods

### Quantitation of Thalidomide (Bioanalytical and Analytical)

### Sample Preparation for Thalidomide (THM) Content/Stent

Extraction of THM from the polymer matrix on the stent is optimized after comparative extraction analysis with methanol and acetonitrile. The solvent with the highest extraction efficiency is selected for subsequent quantitation.

High Pressure liquid chromatographic method for quantitating THM formulation potency

A high performance liquid chromatography (HPLC) method for the determination of thalidomide in rat plasma is modified to quantitate thalidomide coated on the stent (Yang et al. (2005) J. Pharm. Biomed. Anal., 39: 299-304). The chromatographic method uses a reversed-phase Hypersil C18 column and mobile phase consisting of acetonitrile-10 mM ammonium acetate buffer (pH 5.50) (28:72, v/v), at a flow rate of 0.8 ml/min Thalidomide is monitored by ultraviolet detector absorption at 220 nm.

High Pressure liquid chromatographic method for quantitating THM in biological matrices

A high performance liquid chromatography (HPLC) method for the determination of thalidomide in rat plasma is modified to quantitate thalidomide in the mice lung tissue and plasma. THM levels at different times points after dosing are measured as a pilot tissue distribution study as part of the efficacy study to obtain pharmacokinetic and pharmacodynamic correlation. The chromatographic method involves a reversed-phase Hypersil C18 column and mobile phase consisted of acetonitrile-10 mM ammonium acetate buffer (pH 5.50) (28:72, v/v), at a flow rate of 0.8 ml/min

Analytical methodology for PTX and THM-PTX

Sample preparation for PTX content of the stent: Extraction of PTX from the polymer matrix on the stent is optimized after comparative extraction analysis using methanol and acetonitrile. The solvent having the greatest extraction efficiency is selected for subsequent quantitation as described below.

### HPLC Method for Quantitating PTX

The following reversed phase HPLC method is modified from the original (Alltech, Philadelphia, Pa.) to quantitate PTX in the stent and to obtain an in vitro release profile. The retention time of PTX using this method is eight minutes.

The column (53 mm×7 mm; ROCKET (Alltech No. 81174)) comprises ALTIMA Ph, 3 μm (Alltech); the mobile phase comprises A (water) and B (methanol: acetonitrile; 15:85); the gradient is 32% B to 50% B at eight minutes; run time is fifteen minutes at a flow rate of 2.5 ml/min The absorbance of the eluate is monitored at 227 nm.

In addition, a method to simultaneously assay THM and PTX is developed. The method combines key features of both methodologies. Initially quantitation is conducted by assaying samples using the following two methods.

### In Vitro Release Profile of PTX, THM or PTX-THM

This study differentiates slow, medium, and fast release of PTX, THM or PTX-THM formulations for quality control purposes in a dissolution media that is easily available and differentiates changes in release profile. This can be correlated to a 24 hr, 7 day, and 30 day in vivo release rate of the respective drug from a drug eluting stent implanted in the pig airway model.

The stent is placed in a dissolution bath containing 25 ml of dissolution media. The media comprises 0.1-1.0% Polysorbate-80 (a surfactant) in phosphate buffered saline. The percentage of surfactant is determined after some experimental method development. The dissolution media is stirred at 10 rpm per min at 25° C. Aliquots of media from the dissolution bath are sampled at 0, 6 hr, 24 hr, and 48 hr. Samples are assayed using the HPLC method to obtain a release rate profile of PTX on the stent.

### Ex Vivo Release Profile of Drugs

Preserved central airway of pigs are used to study the tissue distribution of an implanted PTX, THM or PTX-THM stent at 0, 2, 6, and 24 hours at 37 C/75% relative humidity incubator. The drug content remaining on the stent and tissue distribution is determined Standard sample extraction procedures are utilized, including tissue homogenization, protein precipitation with acetonitrile and quantitation using HPLC or LC-MS method. The purpose of this study is to correlate data obtained from in vivo tissue distribution studies and to use it as a rapid screening method for formulation optimization in conjunction with an in vitro release method.

### In Vitro Polymer Biodegradation Rates

Tablets of 100 mg of poly(SA), poly(CPH), poly(CPTEG), poly(CPH:SA), poly(CPTEG:CPH) copolymers (the compositions include 20:80 and 50:50 CPH:SA and 20:80 CPH:CPTEG) are melt compressed for 2 min in a Carver Press (Wabash, Ind.) at a pressure of 600 psi and at a temperature just above the melting point of the polymer. Past experience indicates that range of degradation times for these copolymers ranges from ˜1 week to ˜6 months. Then the tablets are placed into 25 ml of phosphate buffer (0.1M, pH 7.4) in an incubator operating at 37° C. and 100 rpm. The buffer is replaced daily. At different time intervals, duplicate samples of tablets are taken out of the buffer for further analysis. The mass loss of the tablets is determined by gravimetric analysis, while the molecular weight loss is monitored by GPC. The surface morphology of the tablets is also monitored using scanning electron microscopy.

### Coating Integrity

The coating integrity of the stent system is evaluated using a microscope and confirmed with a scanning electron microscope to detect cracks and other physical irregularities.

### Formulation and Coating Process Development

FIG. 12 illustrates an exemplary generic formulation and coating process that can be followed during the formulation trials. Generically prepared laser cut nitinol stents with similar dimensions as the Ultraflex stent are coated during the formulation trials process. Once the formulation and process is identified the Ultraflex bare metal stents from Boston Scientific is used as a representative FDA approved self-expanding tracheo-bronchial stent platform.

### Pre-Coating Stent Preparation

The bare metal stents are cleaned sequentially with organic solvents such as acetone, methanol, isopropanol and finally distilled water. These are then dried in an oven at 200° C. for an hour to remove any residual solvents.

### Preparation of Polymeric Drug Formulation

Results from the preformulation screening studies guide the preparation and storage of these formulations. A concentrated solution of the drug or drug combination is prepared in either Dimethysulfoxide, Dimethylacetamide, or ethanol. An aliquot is added to the polymer solution (1-25% w/v) in a suitable organic solvent such as acetone, acetonitrile, or dichloromethane to obtain a drug concentration in the range of 0.1 to 10% w/v.

### Coating of the Polymeric Drug(s) Formulation on the Stent Platform

The polymeric Paclitaxel formulation is coated using either the dip coating process or a spray coating process. Reproducibility, ease of use, efficiency and drug loading determines the process chosen. A laboratory spray coater and dryer is used to coat and dry the drug-polymer formulation on the stent platform.

The stent can be pre-coated or post coated with either an adhering polymer layer or a top polymer coat. Parameters that are varied to engineer drug release profiles are: different drug:polymer ratios, heating and drying temperatures and rate, moisture control, top polymer coat and layered coating with and without drug

### Analytical Testing and Selection of Three Prototype Formulations

Drug eluting stent samples from the formulation and coating trials are evaluated for coating integrity, drug(s) content and in vitro and ex-vivo release rate profiles of the drug. Three prototype formulations bracketing a low, medium and high drug dose and release rate are selected for testing in preclinical efficacy. The bracketed formulation targets a drug load in the range of 0.05-50 mg/stent or higher, a release rate ranging from 1% to 25% released in 24-48 hours. The formulations providing with the highest drug or drug combination dose are then identified.

### Example IV

### Efficacy of Antitumor Activity of Compositions in an Orthotopic Human Lung Cancer H460-GFP Model

### Animals:

Twenty NCr nu/nu male mice, 5-6 weeks old, are used in the assay. Additional mice may be added to the protocol in appropriate numbers to compensate for dead mice right after Surgical orthotopic implantation (SOI). The tumor to be tested is the human lung cancer cell line H460-GFP. Each cage containing experimental animals is clearly marked with a unique way for its group with up to 5 mice per cage. Each mouse has an ear-mark representing the unique marking. All groups are sorted by random selection. Treatment is initiated three days after implantation of the cell line. The test agent is selected from the compositions recited above and is pre-formulated with vehicle. The test agent comprises a polymer and a drug. Animals are anesthetized and trachea is exposed. Less then 20 μl of the test agent (test) or vehicle alone (control) is injected into trachea using a syringe with a 27 G needle. The procedure is summarized in Table 1.

TABLE 1

| Group number | Agent | Dose | Schedule | Route | n |
|---|---|---|---|---|---|
| A | Vehicle | <20 μl | 0.5 h to 5 d | intratracheal | 10 |
| B | Test agent | <20 μl | 0.5 h to 5 d | intratracheal | 10 |

The animals are monitored using GFP imaging twice weekly to check primary tumor and metastasis starting with whenever tumor GFP is captured. Body weights are measured once weekly. The study endpoint is assessed at either approximate four weeks after SOI or when three mice in the control group die, whichever come first and regardless of treatment duration. Animals are examined daily for mortality or signs of morbidity. Morbid animals, especially if death appears imminent, are humanely sacrificed and frozen. All animals including dead animals during the study are checked with open GFP imaging for primary tumor and metastasis at necropsy. Primary tumors are excised and weighed at necropsy. Statistical analysis (Student's t-test; ANOVA) are performed on all animal data.

### Pharmacokinetic Profile

Exposure of drug or drugs is evaluated in the plasma and in the lung tissue as part of the efficacy study. Exposure levels in the plasma and lung tissue at different time points after administration are measured.

Test agents having antitumor activity are then selected for use with the invention.

### Example V

### Pharmacokinetic Studies

Several experiments to address certain fundamental reviewer questions/recommendations related to drug tissue-distribution after stent implantation were conducted. The same experiments would pave the path for preclinical studies and predict the basis of the efficacy of the drug eluting tracheo-bronchial stents. A screening formulation of thalidomide (THX) and polymer was used for the studies and the rat was chosen a preclinical model. The experiments are detailed in the following sections. The summary results are tabulated in Table 2 below:

High levels of THX were found in lung tissue and low levels in plasma after intra-tracheal delivery of THX-polymer formulation at a 12 mg/kg dose (100 μl dosed 3 times) in rats. Data indicated good distribution/diffusion into the lung tissue and vasculature.

High levels of THX were found in tracheo-bronchial and lung tissue after an in situ intra-tracheal infusion (0.5 hr) delivery of THX-polymer formulation at 8 mg/kg dose. (200 μl dose volume) in experimental rats.

TABLE 2
THX levels in plasma, lung, tracheal tissue
after intratracheal in vivo and in situ delivery

| | Dose | | Tissue Levels | | |
|---|---|---|---|---|---|
| | | Volume | Trachea | Lung | Plasma |
| Study | (mg/kg) | (μl) | (μg/100 mg) | (μg/100 mg) | (ng/ml) |

Rat PK study (sampling post 3rd dose)

| | | | | | |
|---|---|---|---|---|---|
| 20 mins | 3 × 4 | 100 | — | 21.80 | 0.19 |
| 40 mins | 3 × 4 | 100 | — | 16.81 | 0.44 |

Rat in situ study

| | | | | | |
|---|---|---|---|---|---|
| Rat 1 | 8 | 200 | 211.21 | 0.58 | — |
| Rat 2 | 8 | 200 | 290.36 | 0.75 | — |

### 1) Tracheo-Bronchial and Lung Tissue Levels of THX

A pilot pharmacokinetic (PK) study was performed to estimate tracheo-bronchial and lung tissue levels after intra-tracheal application of THX-polymer formulation.

### Method:

A 10 mg/ml THX-polymer formulation was delivered (to anesthetized rats) at a dose of 4 mg/Kg at a volume of 100 μl dosed 3 times in 4 rats at 30 min intervals. The animals were anesthetized while dosing. Two of the rats died after the second dose. Blood, tracheo-bronchial and lung tissue was sampled at 20 and 40 min past the third dose. Samples were frozen until processed for LC-MS analysis. The extraction procedure and LC-MS method are described as a separate experiment. Sample extraction was conducted at AraVasc Inc. and the processed samples were shipped to Alta Analytical Laboratory (El Dorado Hills, Calif.) for LC-MS quantitation. Only the plasma and lung tissue were analyzed by LC-MS.

### Results:

THX levels at 20 and 40 mins was 21.80 and 16.81 μg/100 mg of lung tissue respectively THX levels at 20 and 40 mins was 0.19 and 0.44 μg/ml of plasma respectively

### 2) In Situ Intra-Tracheal Infusion

A pilot study of in situ intra-tracheal infusion was performed to estimate tracheal and lung tissue levels after in situ intra-tracheal infusion of THX-polymer formulation.

### Method:

Two rats (immediately after termination) were intra-tracheally infused over 30 mins with a THX-polymer formulation. The dose was 8 mg/kg and volume was 200 μl. After infusion these were wrapped for 2 hours in a warm heating pad. Tracheo-bronchial and lung tissue were harvested after wiping the lumen area of the tracheo-bronchial lumen with a tissue and spatula (to take off any adsorbed drug) at the end of 2 hours. Samples were frozen at −80 C until processed and analyzed by LC-MS. Sample extraction was conducted at AraVasc Inc. The processed samples were analyzed at Alta Analytical laboratories by LC-MS.

### Results:

The levels of THX in the 2 rats were 0.58 and 0.75 μg/100 mg of lung tissue

The levels of THX in the 2 rats were 211.21 and 290.36 μg/100 mg of tracheal tissue

### 3) Tracheal Stent Implantation in Rats

### Method:

A 13 gauge plastic feeding tube is the maximum diameter that will allow passage thru the glottis. Rats were anesthetized using isoflurane at 5% and placed on a slant board and held in place via the upper incisors. A 13 Gauge plastic feeding tube was pre-cut to 2 cm in length. The glottis is viewed from mouth by transilluminating the neck with a fiber-optic light. Using a 18 gauge tube as a stylet, the stent was threaded down the trachea. To keep the tube from sliding down, the trachea was exposed by blunt dissection and a 4-0 silk suture was tied around it. The neck incision was closed using 2 staples. Another rat had the same procedure without tying a suture. Both rats showed difficulty in respiration after the procedure but were breathing better the following morning. The rat with the suture died after 2 days and the rat without the suture has survived for over 3 weeks. Stents were successful implanted and retained for over 3 weeks in the rat model.

### 4) Development of Screening LC-MS Bio-Analytical Method

Method: A preliminary screening LCMS method was developed for quantitating THX in tissues and plasma. The quantitation method used positive ion mode MRM scan at protonated molecular ion of 259.0 and the product ion at 186.4 ion of 259. The method conditions and parameters are listed in table below. The equipment used was a SCIEX PE 3000. The method is summarized in Table 3.

TABLE 3
LC-MS method for the quantitation of THX in tissues and plasma.

| Parameters | Description | |
|---|---|---|
| Column | ACE 5 Phenyl, 100 × 2.1 mm | |
| | (Advanced Chromatography Technologies) | |
| Mobile Phase | A: 0.1% Formic acid B: 0.1% Formic acid in Acetonitrile | |
| | Time | Mobile Phase A:B |
| Gradient | 0 time: | 80:20 (A:B) |
| | 0.5 min: | 20:80 |
| | 2.0 min: | 20:80 |
| | 3.5 min: | 80:20 |

| | |
|---|---|
| Flow rate | 0.5 ml/min |
| Detection | Multiple Reactions Monitoring Mode: TurboIonSpray in |
| | positive ion monitoring SCIEX PE 3000 |
| | Positive MRM at protonated 259.0 and the product ion at |
| | 186.4 |

### Tissue and Plasma Extraction:

The tissue was ground (glass-glass homogenizer) and extracted with 70:30 0.1% formic acid in acetonitrile and 0.1% formic acid in water at a 1:3 ratio of tissue to solvent. Plasma is extracted with the same solvent as the tissue at a 1:2 ratio of plasma to solvent. The supernatant extract was subject to LCMS analysis.

Results: A screening LC-MS method was developed for the quantitation of THX in the concentration range of 50-30,000 ng/ml

### Example VI

### Preclinical Formulation

### Method:

THX was dissolved in dimethylsulfoxide (1%); cosolvents-solubilizers polyethylene-glycol-300 (PEG-300) (20% v/v), polysorbate-80 (2.5%), Phospholipids (5%), polyanhydride CPH:SA (20:80) polymer (10%) dissolved in ethanol were mixed and vortexed for homogeneity. The formulation was ‘QSed’ (made to volume) with PBS. A viscous but syringeable suspension formulation at 10 mg/ml THX strength was obtained and used for POC PK studies.

Other exemplary modes of making and using the invention are disclosed in US provisional patent application from which the instant application claims priority, US60/745,834, which is herein incorporated by reference in its entirety.

### Example VII

### HPLC Quantitation

a. Reverse phase HPLC method for the simultaneous quantitation of PTX, THX, FLX and ICG was modified to accommodate analysis of HCFU (Carmofur) and (CPX) Capecitabine. These data-driven modifications to the method were required as the project progressed and a strategic decision was made to include HCFU instead of 5FU as one of the ETDS drug components. Table 4 lists the salient features of the method.

TABLE 4
HPLC assay Method for Analysis of FLX, THX, PTX, HCFU and
ICG and Validation Summary

| Attributes | Description |
|---|---|
| Column | C18, 5u 250 mm × 4.6 mm |
| Flow Rate | 1.2 ml/min |
| Mobile | A: 5 mM KH₂PO₄: ACN (90:10) B: 5 mM KH₂PO₄: ACN |
| Phase | (40:60) |
| Gradient | 22 min gradient |
| Detection | 230 nm PTX and THX; 780 nm ICG; 254 nm FLX |
| Retention | FLX, THX, ICG, HCFU, CPC, PTX: 3.1, 6, 15.2, 15.9, 10.7, |
| Time | 17.4 mins ± 10%, |
| Linearity | 0.8-100 μg/mL, PTX, THX; |
| LOQ | 0.8 μg/mL, PTX, THX; 0.6 ug/ml HCFU, 0.2 μg/mL, ICG |
| LOD | 0.4 μg/mL, PTX, THX; 0.2 ug/ml HCFU, 0.05 μg/mL, ICG |
| Specificity | Method is specific to FLX, PTX, THX, HCFU and ICG |
| Stability | Standard and samples stable for >48 hours at room |
| | temperature |
| Recovery | >95% of nominal concetration |

### Example VIII

### Drug Release Profile A

Data-driven modifications to the drug-release profile method were made to accommodate HCFU. Table 5 lists the salient features of the modified method. The quantitation method is similar to the one used for assay. Data-driven modifications are made for the different ETDS product strengths. This includes temperature of the bath, sampling periods, duration of drug-release. Based on the results 1-2 hour sampling interval for the first 12 hours and 24-hour sampling over a 3 to 7-day duration at room temperature has been used for the drug-release studies.

TABLE 5
Drug-Dye-Release Method for Analysis of FLX, THX, PT HCFU and
ICG and Validation Summary

| Attributes | Description |
|---|---|
| Chromatography | Same as in Table 32 |
| Release Media | 0.5% Solutol, 25 mM Phosphate buffer pH 4 |
| Linearity | 0.8-100 μg/mL, FLX, PTX, THX; 5.0-100ug for |
| | HCFU r² = 0.999 |
| LOQ | 0.8 μg/mL, FLX, PTX, THX; 0.6 ug/ml HCFU, |
| | 0.2 μg/mL, ICG |
| LOD | 0.4 μg/mL, FLX, PTX, THX; 0.05 μg/mL, 0.2 ug/ml |
| | HCFU, ICG |
| Specificity | Method is specific to FLX, HCFU, CPC PTX, THX |
| | and ICG |
| Recovery | >95% of nominal concentration (HCFU had to be |
| | analyzed within 2 hrs-optimal stability at pH3) |
| Stability | Standard and samples stable for >48 h at room |
| | temperature (RT) |

### Example IX

### Drug Release Profile B

Formulation trials of prototype ETDS with 5-fluorouracil (5FU) and THX or PTX and attempts to slow down the release of 5FU were unsuccessful. Polymers such as PVP (polyvinyl pyrrolidine) and polyanhydride (CPH: SA) at different drug: polymer levels such as 1:1, 1:3 and 1:5 were tested. Table 6 shows the results of dissolution profile of the 5FU released from a prototype stent.

TABLE 6
Drug-release profile of 5FU

| | % Recovery of FLX (5FU) | | |
|---|---|---|---|
| | in drug release media | | |
| Drug | 2 hr | 8 hr | 24 hr |
| 5FU:THX | >80% | >80% | >80% |
| 5FU:THX:PVP (1:1:1) | >80% | >80% | >80% |
| 5FU:THX:PVP (1:1:5) | >80% | >80% | >80% |
| 5FU:THX:PVP (1:1:5) + | >60-80% | >60-80% | >60-80% |
| overcoat with 5 PVP | | | |
| 5FU:THX:CPH:SA (1:1:5) + | >80% | >80% | >80% |
| overcoat with 5 PVP | | | |

*THX was used as marker since its release profile is well characterized

### Example X

### Evaluation of HCFU and CPX

Formulation trials of prototype ETDS with alternative drug HCFU and CPX were successfully coated on ETDS with an increased ETDS residence time. The polyanhydride polymer CPH: SA slowed down release of HCFU and CPX. HCFU has been chosen as the analogue of choice while CPX is being evaluated as a backup to HCFU. Drug release profile of HCFU along with THX or PTX was evaluated with and without polymer (CPH: SA).

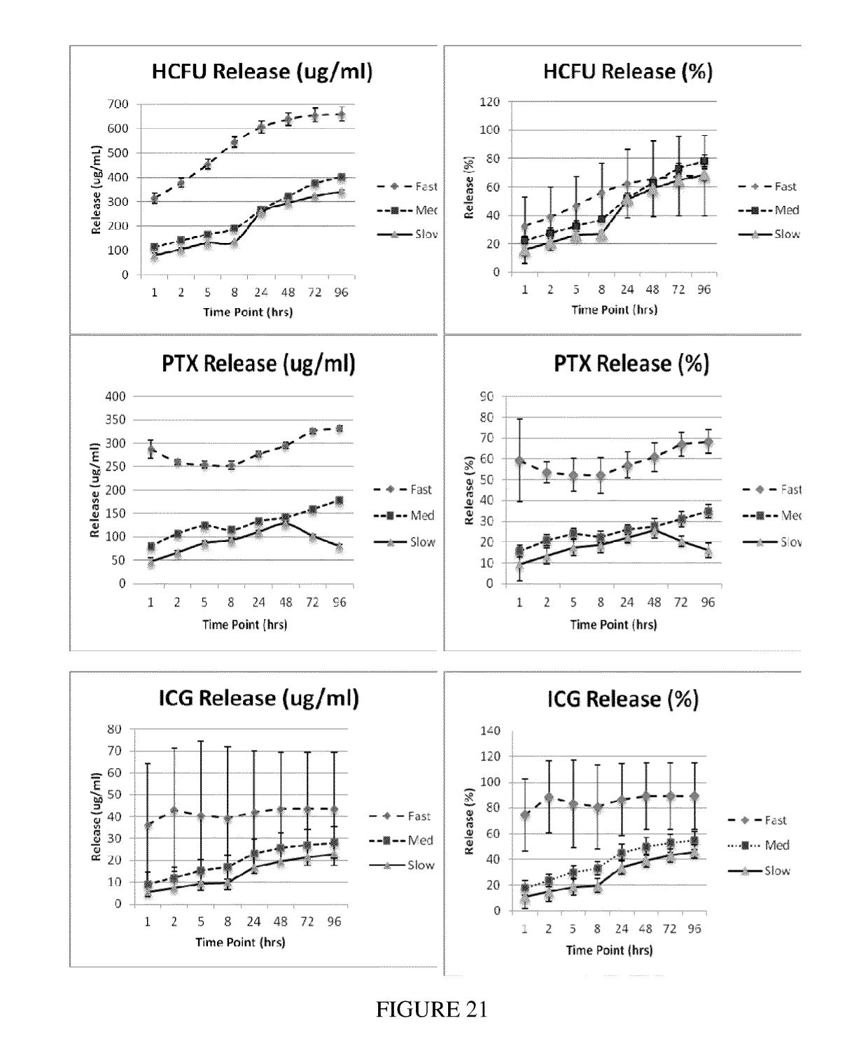

Definitive ETDS trials HCFU-PTX-ICG: Formulation trials were focused on engineering release rate of drug-dye combination HCFU-PTX and ICG. Results indicate that both HCFU and ICG are stabilized by the nanoporous stent and are released from the stent for over 72 hours without the polymer. HCFU is unstable in the dissolution media at pH>3 while ICG is unstable at pH<3. A suboptimal but workable pH 4 was chosen to evaluate the release profile. The polymer substantially slowed drug-dye release rates. A representative drug-release profile of fast, medium and slow release rates are shown in FIG. 21. The following are the highlights of the trials:

### Formulations:

The concentration of ICG was 1/10^{th} that of HCFU and PTX. Concentration of PTX and HCFU ranged from 1-20 mg/ml and the Drug: polymer ratio ranged from 1:0-1:5.

### Drug Load:

1-10 mg PTX, HCFU/ETDS unit; 50 mg HCFU, PTX load per ETDS with a total load of 100 mg was demonstrated. Batch analysis of representative ETDS shown in Table 8.

### Correlations:

The HPLC assay/ETDS values correlated within 100±20% with the weight of the theranostic coat/ETDS; strength of the actives can be projected from a simple, non-destructive coat-weight/ETDS.

### Release Profile:

Fast, medium and slow drug-release formulations were identified for ICG, PTX and HCFU (FIG. 21 compares fast, medium and slow release profiles for HCFU, PTX and ICG, respectively)

### Process:

Semi-automated coating process and equipment identified. This includes an atomized spray coating process with intermittent drying on a rotating mandrel.

TABLE 7
Solubility Results for FLX, PTX, THX, ICG, HCFU, CPX and Polymer (CPH:SA)

| | Solubility (mg/mL) | | | | | |
|---|---|---|---|---|---|---|
| | | | | | Dissolution | |
| | | | | | Media | |
| Analyte | Acetonitrile | DMF | DMSO | THF | Media | Water |
| PTX | 44.3 | 154.2 | 27.2 | 41.7 | 0.05-0.06 | >20 |
| THX | 0.79 | 75.5 | 56.0 | 3.34 | 3.34 | 1.5 |
| ICG | 0.03 | 46.2 | 62.2 | <0.5 | >5 | >5 |
| CPH:SA | 0.26 | 55.83 | 11.4 | 11.96 | <0.1 | <0.1 |
| FLX | 1.81 | 45.8 | 59 | 42.1 | 42.1 | >20 |
| HCFU | >0.4 | >50 | >50 | >50 | 0.05-0.06 | 0.02-0.03 |
| CPX | >0.4 | >50 | >50 | >50 | >10 | >10 |

HCFU, CFX Solubility - Monitored by visual clarity; solubility of all analytes were >20 mg/ml in 1:3 DMF:THF and was used as the spray media.

TABLE 8
6. Batch Analysis of Fast release ETDS

| Assay | Test Results | | |
|---|---|---|---|
| FR ETDS | ICG | HCFU | PTX |
| Assay by HPLC | 0.59 ± 0.07 | 5.1 ± 0.5 | 4.0 ± 0.3 |
| (mg/ETDS (n = 3) mg | | | |
| Assay by Weight | 0.53 ± 0.02 | 5.3 ± 0.8 | 5.2 ± 0.7 |
| (mg/ETDS (n = 3) mg | | | |
| Assay by HPLC | — | 68.5 ± 3.5 | 53.5 ± 2.1 |
| (mg/ETDS (n = 3) mg | | | |
| Assay by Weight | — | 56.7 ± 2.4 | 49.2 ± 1.5 |
| (mg/ETDS (n = 3) mg | | | |

### Example XI

### Demonstration of Proof-of-Concept Theranostic Activity in a Mouse Tumor Xenograft Model and Identify the Most Efficacious Drug-Dye Combination

The approach was to implant the theranostic coated preclinical stent that would provide an efficacious dose for tumor growth inhibition. Tumor visualization was conducted using two modes, near infra-red fluorescence imaging (ICG) (750-900 nm) along with GFP fluorescent tag (395-509 nm). As the project progressed, several efficacy studies were required due to change of drug combinations. The first one identified the more efficacious drug combination PTX-FLX and was compared to THX-PTX. Due to technical limitations of coating FLX on the ETDS, a lipophilic analogue HCFU was selected to replace FLX. The second study confirmed efficacy of the new combination. A third in vivo study was conducted to demonstrate theranostic attributes of ICG for tumor visualization.

Due to the time constraints and cost considerations the near infra-red fluorescence imaging system was not available at the same time as the IVIS imaging system that monitored the GFP-tag excitation wavelength at 395 nm, and an emission wavelength set at 509 am. To overcome this the study was conducted as two proof-of-concept studies. The first included implantation of the to visualize the distribution of ICC in the mouse tumor with high sensitivity and selectivity. The other included POC efficacy study showing tumor growth inhibitor by the preclinical stent ETDS monitored using fluorescent GFP-tag.

### Results and Discussion

### POC Study 1—In Vivo Visualization of the Tumor by ICG Theranostic Coat:

Methodology testing including visualization of the theranostic stents implanted in tumor-bearing mice, using whole body imaging system has been completed. Preclinical ETDS coated with 5 μg ICG were implanted subcutaneously in tumor-bearing mice (N=5). The ETDS was ejected in 3 of the mice between 2-24 hr post implantation. The ETDS was retained in the other two for two weeks and was explanted at necropsy. The study was designed to evaluate the distribution of ICG from the implant into the tumor.

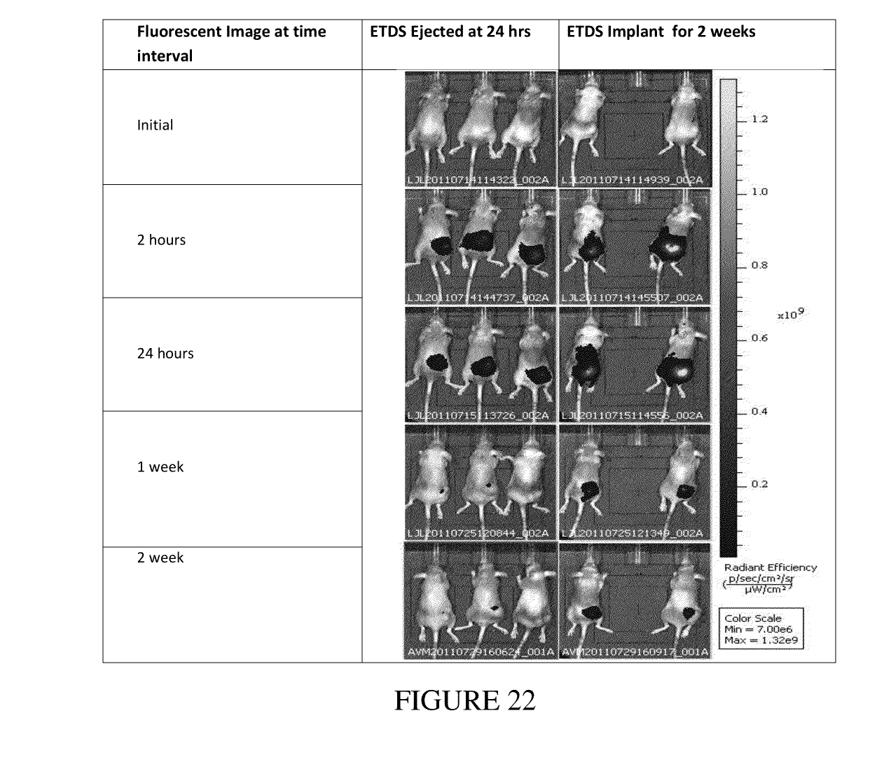

Whole body near infra-red images were taken at 2 hr, 24 hr, 1 and 2 weeks post implantation with an Olympus OV100 imaging system. Fluorescence images of the animals were acquired using the filter setting pre-set for Indocyanine Green with a background wavelength at 665-695 nm, an excitation wavelength at 710-760 nm, and an emission wavelength set at 810-875 nm. The tumor size was 10 mm×10 mm. Dramatic tumor visualization results were observed (FIG. 22). The theranostic coat selectively lit up the tumor, enabling tumor visualization with high specificity and sensitivity two weeks post implantation. The high in-vivo protein binding of ICG (about 95%), limits it largely to intravascular compartment and forms the basis of this application.

### Example XII

### Efficacy Study with Drug Combinations

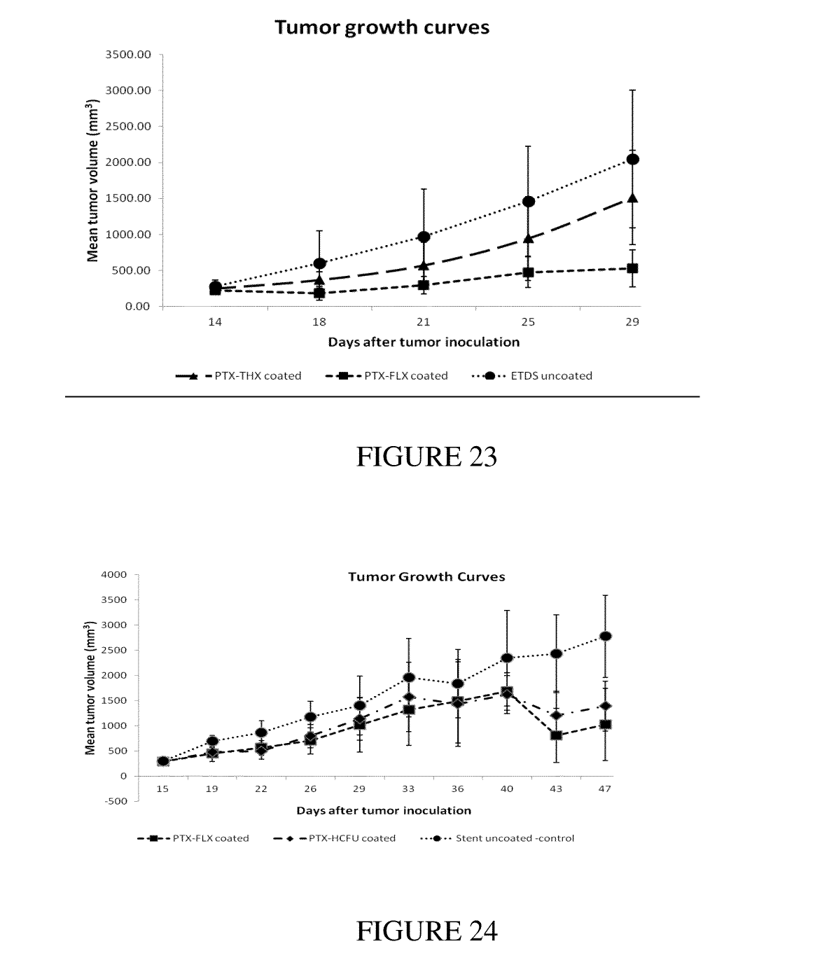

An efficacy study was conducted with the fast-release formulations with both drug combinations PTX-FLX and PTX-THX. The fast release mini-ETDS ensured the highest dose delivery. Coated or uncoated fast release preclinical ETDS were implanted in contact with tumor (300 mm³), 14 days after inoculation of human esophageal-gastric cancer line NUGC4-GFP into nude mice. Tumor growth inhibition was seen 14 days post-implantation (FIG. 23). The total drug dose was estimated to be 125 ug±25 each of FLX-PTX or THX and PTX respectively. The projected in vivo dose/day based on an assumption of constant release after the initial ‘burst-effect’, is about 0.4 mg/kg/day. Similar body weight between the groups suggested that ETDS had no obvious systemic toxicity.

### Example XIII

### ETDS Efficacy in a Subcutaneous NUGC-GFP Cancer Cell Line Xenograft Model at Less than 1/10^{th} the Systemic Dose (PTX-FLX Compared with PTX-HCFU)

A repeat efficacy study to verify efficacy of the alternative lipophilic drug combination was conducted with the fast-release formulations with both drug combinations PTX-FLX and PTX-HCFU.

The fast release mini-ETDS ensured the highest dose delivery. Coated or uncoated fast release preclinical ETDS were implanted in contact with tumor (300 mm³), 14 days after inoculation of human esophageal-gastric cancer line NUGC4-GFP into nude mice. Tumor growth inhibition was evaluated over 47 day period post-implantation (FIG. 24). The total drug dose was estimated to be 115±20 ug each of FLX-PTX or 115±20 ug each of HCFU and PTX respectively. The projected in vivo dose/day based on an assumption of constant release after the initial ‘burst-effect’, is about 0.45 mg/kg/day. Similar body weight between the groups suggested that ETDS had no obvious systemic toxicity.

The three studies together have demonstrated proof-of concept theranostic efficacy showing both selective and sensitive visualization of the tumor by the fast-release formulation and tumor growth inhibition. Both drug combinations PTX-FLX and with PTX-HCFU exhibit activity. The ICG coated ETDS demonstrated tumor visualization by near infra red fluorescence whole body imaging. The third study comparing PTX-HCFU and PTX-FLX was carried on for a longer duration (47 days) and animals were terminated when the tumor size in animals grew to >2800 mm³ As a result, although not enough animals were present on the study termination day to conduct a statistical analysis, results indicate efficacious trend with both PTX-FLX and PTX-HCFU. It was exciting to see that some animals with grown tumors saw a complete reduction in size.

The projected dose released by the fast-release formulation of PTX-HCFU and PTX-FLX assuming constant release (after the initial burst effect) is projected to be 0.43 mg/kg/day. This dose is < 1/10^{th the systemic dose with either HCFU or PTX. In vivo drug distribution in tumor bearing and naïve mice are discussed in next section. This is clearly an unexpectedly superior result.}

### Example XIV

### Drug Distribution Studies in Tumor-Bearing and Naïve Mice to Establish In Vitro In Vivo Correlations of ETDS Formulations

A drug-dye distribution study was conducted in naïve mice. Plasma, skin tissue at implant site and skin tissue at contralateral site was sampled at defined intervals ranging from 2 hours-28 days. Similarly, plasma and tumor tissue was sampled at efficacy study termination at either 28 or ???? days post implantation.

Drug-dye exposure levels in each of these biological samples was quantitated by LC-MS to obtain correlations between efficacious levels of exposure and release profile in naïve mice.

### Example XV

### Drug Distribution 28-Day PK Study in Mice

An in vivo 28-day PK and drug distribution studies was conducted to understand drug distribution and the release profile/duration of drug-released from the different ETDS.

Nine mice were subcutaneously implanted with the same ETDS as implanted in the efficacy study. Blood was sampled from the mice on day 4 (non-terminal bleeds), 7, 14, and 21 days. Stents were explanted and skin tissue at the implantation site and contra-lateral site was excised on day 7, day 14, and day 28 upon euthanasia at each of the time points. Three animals were terminated at each of these time points. Drug distribution in the plasma and skin tissue was analyzed by LC-MS. The preclinical stents were explanted and assayed for residual drug. Results of the drug distribution analysis are outlined in Table 9.

TABLE 9
Drug Distribution in the naïve mice implanted with ETDS formulations

| | | | | | | | Day 0 ETDS or | | |
|---|---|---|---|---|---|---|---|---|---|
| | Plasma | | | Tissue Conc. (ng/g) | | | Explanted ETDS | | |
| | Conc. (ng/mL) | | | site of implant | | | Conc. (μg) | | |
| | ICG | THX | PTX | ICG | THX | PTX | ICG | THX | PTX |
| Day0-FR-A | — | — | — | — | — | — | 2.5 | 76.33 | 79.75 |
| Day0-FR-B | — | — | — | — | — | — | 3.5 | 64.91 | 69.39 |
| Day0-FR-C | — | — | — | — | — | — | 5.8 | 65.23 | 68.23 |
| Day4-FR-1 | 3.89 | 0.95 | BLOQ | 15.3 | 387 | 18000 | — | — | — |
| Day4-FR-2 | 2.39 | 3.26 | BLOQ | 32.4 | 338 | 6270 | — | — | — |
| Day4-FR-3 | 3.76 | 0.00 | BLOQ | 20.9 | 6410 | 6990 | — | — | — |
| Day7-FR-4 | 3.23 | 5.42 | BLOQ | 13.2 | 22.5 | 22000 | — | — | — |
| Day7-FR-5 | 0 | 7.27 | BLOQ | 12.4 | 66.5 | 10300 | — | — | — |
| Day7-FR-6 | 2.26 | 9.27 | BLOQ | 35.9 | 18.4 | 47000 | — | — | — |
| Day14-FR-7 | 2.23 | — | BLOQ | 12.2 | 30.9 | 16500 | — | 0.21 | 12.30 |
| Day14-FR-8 | 0 | 2.26 | BLOQ | 13.1 | 15.9 | 10400 | — | 0.31 | 14.82 |
| Day14-FR-9 | 0 | 7.55 | BLOQ | 14 | 16.8 | 21600 | — | 0.10 | 15.13 |
| Day28-FR-10 | 2.39 | 9.03 | BLOQ | 12.1 | 20.2 | 18900 | — | — | — |
| Day28-FR-11 | 2.24 | 13.7 | BLOQ | 11.8 | 34.0 | 12800 | — | — | — |
| Day28-FR-12 | 2.26 | 7.79 | BLOQ | 11.9 | 15.8 | 10600 | — | — | — |

Additional data is being collected and will be presented in the final reported.
LLOQ = 5 ng/mL (Plasma); LLOQ in subcutaneous tissue = 25 (ICG), 10(PTX), 5(HCFU) ng/m;

TABLE 10
Drug Distribution in the tumor-bearing mice and naïve implanted with
ETDS formulations
Drug Levels-HCFU and PTX in tumor bearing and naïve mice

| | *HCFU | | | PTX | | |
|---|---|---|---|---|---|---|
| Drug Exposure (n = 8) | ng/ml | | SD | ng/gm | | SD |
| Efficacy Study | | | | | | |
| Plasma (day -28-47) (n = 8) | 58.97 | | 54.2 | 1.53 | | 0.3 |
| Tumor Tissue (day -28-47) | 54.1 | | 51.9 | 9,668 | | 1,208 |
| Drug distribution | | | | | | |
| study -Naïve mice | | | | | | |
| Plasma (day -28) n = 3 | 48.2 | | 23.1 | BLOQ | | BLOQ |
| Subcutaneous Tissue | 43.3 | | 39.5 | 12,118 | | 4300 |
| (day -28) n = 3 | | | | | | |
| Explanted ETDS (n = 3) | 2.1 | ug | 0.5 | 15.4 | ug | 5.1 |
| (Residual - efficacy) | | | | | | |

*HCFU has stability issues during processing: values are variable and may be underestimated; ETDS 125 ± 25 ug total drug (1:1)

There is a gradient distribution of drugs from the implants in the in vivo setting. High levels of 5FU were seen in plasma and at the implanted site. ICG remained confined to the site of implantation. PTX, the more lipophilic drug remains more localized at the implantation site. ICG also remained localized at the implantation site suggesting preferentially distribution at the local tumor site. Evidence of this has been demonstrated in tumor-bearing mice in the theranostic whole body imaging study with ICG-ETDS. From the drug distribution study conducted in the naïve mice implanted with different combinations of drugs, PTX-THX-ICG or PTX-HCFU-ICG the drugs and the labile dye ICG are released for the duration tested (4 weeks). Correlating the results from the efficacy study (FIG. 22, 22), drug distribution (Table 9) in the naïve mice to the tumor-bearing mice on terminal day (ranging from D-28 to 47) (Table 10 steady date distribution) in tumor-bearing mice, it appears that the fast release ETDS delivers efficacious drug loads. The efficacious drug dose is 0.43 mg/kg/day.

Table 9 lists the drug exposure (THX, ICG and PTX) in plasma and skin tissue in naïve mice implanted with mini-ETDS. Data shows a gradient diffusion of drug from the implant site. Table 10 compares the exposure level of HCFU and PTX-implant in naïve and tumor-bearing mice. The plasma and tumor tissue exposure at steady state (termination dataday—28-47) correspond to an average of 59 ng/ml and 54 ng/gm HCFU respectively. Average levels of PTX detected in plasma are 1.5 ng/ml. High levels of PTX (9,668 ng/gm) were localized in tumor tissue. The skin tissue and plasma exposure in naïve mice at a similar dose on day 28 are comparable. Based on these results, we could potentially use pharmacokinetics in naïve mice to identify dose vs exposure relationship. Finally, the fast release mini ETDS releases drug for at least 28 days in the in vivo setting. The prototype ETDS fast release is projected to release drugs in vivo for 30 days. The medium and slow release ETDS are projected to release over a 1-3 month period.

The LCMS analysis was conducted using validated bioanalytical methods discussed below except for HCFU. HCFU has limited stability at physiological pH and validation related to recovery was not completed. Samples were processed and analyzed immediately. Values may be underestimated.

### Example XVI

### Bio-Analytical High Pressure Liquid Chromatography-Mass Spectrometry (LC-MS) Method

Table 11 outlines the method validation results for the analysis of THX, PTX and ICG in plasma/skin tissue samples.

TABLE 11
Bioanalytical Method Summary

I. Instrumental:

| | |
|---|---|
| a. | API-4000 Mass Spectrometer. ESI positive, MRM Scan |
| b. | Shimadzu HPLC/CTC Auto-sampler with Waters C8 column (2.1 × 50 mm, 3.5 μm) |
| c. | Mobile Phase A: 0.1% Formic acid, 5 mM NH4Ac in water |
| d. | Mobile Phase B: 0.1% Formic acid in Acetonitrile |
| e. | 10 μL of sample was injected |

II. Sample Preparation

| a. | Standard and QC Samples: | |
|---|---|---|
| | i. | 1 mg/mL stock solution was prepared in DMSO/ACN(1/1). |
| | ii. | Make standard working solutions in 50% ACN with the stock solution. The concentrations of |
| | | the working solutions were 20000, 10000, 5000, 2000, 1000, 500, 200, 100 and 50 ng/mL. |
| | iii. | Spiked 10 μL of the working solutions into 90 μL blank mouse plasma or control tissue |
| | | samples homogenized and vortex them |
| | iv. | Add 300 μL of internal standard solution (Verapamil, 20 ng/mL in 100% ACN), vortex and |
| | | centrifuge |
| | v. | Transfer the supernatant into a HPLC injection plate. |
| | vi. | The standard samples were at 5, 10, 20, 50, 100, 200, 500, 1000 and 2000 ng/mL. The QC |
| | | samples were at 5 or 10 (LQC), 50 (MQC) and 500 (HQC) ng/mL. |
| | vii. | ICG, THX and PTX were combined in a set of Standard and QC samples. |

| | |
|---|---|
| b. | In life samples: |
| | i. 1 mL of water was added into tissue samples |
| | ii. Homogenized the tissue samples. |
| | iii. Added 300 μL of internal standard solution to the 100 μL of plasma or homogenized tissue |
| | samples, vortex, and centrifuged |
| | iv. Transferred the supernatant into a 96-well injection plate |

### Validation Summary and Conclusions

The results of the method validation are listed in Tables 12, 13, and 14, and summarized below:

1. The LLOQ of the three test compounds in mouse plasma is 5 ng/mL with <20% of % RE and % CV.
2. The linear range of the calibration standards for the three compounds is 5-2000 ng/mL with good linear regression (R>0.9936)
3. The intraday accuracy and precision of LLOQ, LQC, MQC and HQC samples met the acceptance criteria (% RE and % CV<±15% for QCs or 20% for LLOQ)
4. The sample extraction recovery of the three compounds in mouse plasma is above 87% at three test concentration levels.
5. There is no interference in plasma to the three analytes. The method is selective.
6. There is no carryover in this method. The method is qualified to use as a non-GLP bioanalysis study.

TABLE 12
Back-Calculated Concentrations of Calibration Standard Samples

| Sample | Nominal Conc. | ICG | | | | THX | | | | PTX | | | |
|---|---|---|---|---|---|---|---|---|---|---|---|---|---|
| ID | (ng/mL) | 1 | 2 | mean | % RE | 1 | 2 | mean | % RE | 1 | 2 | mean | % RE |
| 1 | 0 | 0 | 0 | 0 | 0 | 0 | 0 | 0 | 0 | 0 | 0 | 0 | 0 |
| 2 | 5 | 5.47 | 5.28 | 5.38 | 7.5 | 3.99 | 6.11 | 5.1 | 1 | 4.95 | 4.5 | 4.7 | −5.3 |
| 3 | 10 | 8.53 | 8.78 | 8.66 | −13.5 | 9.37 | 5.88 | 7.6 | −23.8 | 13.9 | 11.3 | 12.6 | 26 |
| 5 | 50 | 55.8 | 53.5 | 54.7 | 9.3 | 53.2 | 46.8 | 50 | 0 | 45.8 | 46.7 | 46.3 | −7.5 |
| 6 | 100 | 100 | 104 | 102 | 2 | 104 | 91.5 | 97.8 | −2.25 | 97.2 | 85.2 | 91.2 | −8.8 |
| 7 | 200 | 225 | 208 | 217 | 8.25 | 229 | 177 | 203 | 1.5 | 227 | 175 | 201 | 0.5 |
| 8 | 500 | 489 | 466 | 478 | −4.5 | 501 | 470 | 486 | −2.9 | 539 | 470 | 505 | 0.9 |
| 9 | 1000 | 986 | 1070 | 1028 | 2.8 | 1030 | 898 | 964 | −3.6 | 958 | 900 | 929 | −7.1 |
| 10 | 2000 | 1980 | 1870 | 1925 | −3.75 | 2300 | 1970 | 2135 | 6.75 | 2380 | 1920 | 2150 | 7.5 |

R (ICG) = 0.9936,
R (THX) = 0.9978,
R (PTX) = 0.9946

TABLE 13
Intraday LLOQ, Accuracy and Precision

| | Nominal Concentration (ng/mL) | | | | | | | | | | | |
|---|---|---|---|---|---|---|---|---|---|---|---|---|
| Sample | ICG | | | | THX | | | | PTX | | | |
| ID | 10 | 50 | 500 | 5(LLOQ) | 10 | 50 | 500 | 5(LLOQ) | 10 | 50 | 500 | 5(LLOQ) |
| 1 | 8.6 | 46.5 | 467.0 | 4.3 | 9.5 | 50.4 | 483.0 | 4.0 | 10.2 | 46.7 | 418.0 | 9.2 |
| 2 | 8.3 | 44.3 | 474.0 | 3.8 | 8.3 | 49.5 | 526.0 | 5.3 | 9.3 | 45.3 | 418.0 | 4.5 |
| 3 | 8.7 | 47.2 | 426.0 | 5.0 | 9.8 | 48.2 | 484.0 | 5.4 | 7.2 | 48.2 | 428.0 | 4.9 |
| 4 | 8.9 | 46.6 | 506.0 | 3.8 | 8.0 | 49.9 | 524.0 | 6.1 | 10.2 | 43.4 | 460.0 | 6.3 |
| 5 | 8.1 | 44.6 | 474.0 | 4.9 | 7.9 | 50.7 | 499.0 | 4.1 | 11.3 | 50.6 | 432.0 | 5.9 |
| 6 | 9.5 | 50.9 | 391.0 | 4.3 | 7.3 | 53.9 | 485.0 | 5.3 | 10.1 | 46.0 | 416.0 | 4.2 |
| mean | 8.7 | 46.7 | 456.0 | 4.3 | 8.5 | 50.4 | 500.0 | 5.0 | 9.7 | 46.7 | 429.0 | 5.8 |
| SD | 0.5 | 2.4 | 41.0 | 0.5 | 1.0 | 1.9 | 20.1 | 0.8 | 1.4 | 2.5 | 16.6 | 1.9 |
| % RE | −4.2 | −10.4 | −8.7 | −13.5 | −4.8 | 1.4 | 0.5 | 0.4 | −0.9 | −10.3 | −14.3 | 15.9 |
| % CV | 5.6 | 5.1 | 9.0 | 12.6 | 11.6 | 3.8 | 4.0 | 16.1 | 14.4 | 5.3 | 3.9 | 32.1 |

TABLE 14
Recovery of Analytes in the Sample Purification Process

| | | ICG | | | THX | | | PTX | | |
|---|---|---|---|---|---|---|---|---|---|---|
| | Spiked | Detected | | | Detected | | | Detected | | |
| Sample | Conc. | Conc. (ng/mL) | | Recovery | Conc. (ng/mL) | | Recovery | Conc. (ng/mL) | | Recovery |
| ID | (ng/mL) | Before | After | (%) | Before | After | (%) | Before | After | (%) |
| 1 | 5 | 3.75 | 5.08 | | 6.05 | 4.43 | | 6.27 | 6.34 | |
| 2 | | 4.88 | 4.81 | | 4.05 | 4.13 | | 5.85 | 5.34 | |
| 3 | | 4.28 | | | 5.27 | | | 4.15 | | |
| mean | | 4.3 | 4.95 | 87 | 5.12 | 4.28 | 120 | 5.42 | 5.84 | 92.9 |
| 1 | 50 | 46.6 | 47.8 | | 49.9 | 47 | | 43.4 | 44 | |
| 2 | | 44.6 | 44.5 | | 50.7 | 48.3 | | 50.6 | 44.3 | |
| 3 | | 50.9 | | | 53.9 | | | 46 | | |
| mean | | 47.4 | 46 | 102.6 | 51.5 | 47.7 | 108 | 46.7 | 44.2 | 106 |
| 1 | 500 | 506 | 576 | | 524 | 529 | | 460 | 426 | |
| 2 | | 474 | 413 | | 499 | 469 | | 432 | 367 | |
| 3 | | 391 | | | 485 | | | 416 | | |

| | | | | | | | | | |
|---|---|---|---|---|---|---|---|---|---|
| mean | 457 | 495 | 92.4 | 503 | 499 | 101 | 436 | 397 | 110 |

While particular embodiments of the present invention have been described, it will be obvious to those skilled in the art that changes and modifications can be made without departing from this invention in its broader aspects and, therefore, the appended claims are to encompass within their scope all such changes and modifications as fall within the true spirit and scope of this invention.

### REFERENCES

1. FDA Approves New Combination Therapy for Lung Cancer. FDA News. U.S. FDA; 2006, www.FDA.gov.
2. Genentech. A significant survival benefit: comments from leading oncologists. Avastin. www.avastin.com 2009.
3. Ramsey S D et.al. Does economics matter when treating advanced NSCLC. The Oncologist. 2002; 7:179-80.
4. Kolata, G., & Pollack, A. Costly cancer drug offers hope, but also a dilemma. Health. New York Times; 2009.
5. T. G. Roberts, Jr., et al. Choosing chemotherapy for lung cancer based on cost: Not yet. The Oncologist. 2002; 7(3):177-8.
6. B. Pettiford & R. J. Landreneau. Endobronchial Stents and Bronchial Sparing Surgery in the Management of Lung Cancer. Revista Del Instituto Nacional de Enfermedades Respiratorias Ismael Cosio Villegas. 2007; 20(1): 33-41.
7. M. E. Lund, R. Garland, & A. Ernst. Airway Stenting. Chest. 2007; 131(2):579-587.
8. Cavaliere S et al. Laser Bronchoscopy. Mathur P N et.al (eds): Interventional. Bronchoscopy. Prog Respir Res. Karger. 2000; 30:108-119.
9. M. Noppen, et al. Interventional. Bronchoscopy: 5-year experience at Vrije Universiteit Brussel (AZ-VUB). Acta Clinica Belgica. 1997; 52(6):371-380.
10. Venuta F., et al. Nd:YAG lasr resction LC invading the airway-bridge to surgery &palliative treatment. Ann Thorac Surg. 2002; 74:995-8.
11. Bolliger Soler M., et al. Combination endobronchial and conventional therapy possibilities in inoperable central lung tumors. Schweiz Med. Wochenschr. 1995; 125:1052-1059.
12. Stohr S. & Bolliger C T. Stents in the management of malignant airway obstruction. Monaldi Arch Chest Dis. 1999; 264-8.
13. Brichet A., et al. Multidisciplinary approach to management of postintubation tracheal stenoses. Eur Respir J. 1999; 13:888-93.
14. Mughal M M, et al. Short-term deployment of self-expanding metallic stents facilitates healing of bronchial dehiscence. Am J Respir Crit Care Med. 2005; 172:768-771.
15. Dineen K M, Jantz M A, & Silvestri G A. Tracheobronchial stents. J Bronchol. 2002; 9:127-137.
16. Freitag L, et al. Clinical evaluation of bifurcated dynamic airway stent:—5-year experience—135 patients. Thor Card Surg. 1997; 45:6-12.
17. Seymour C W, et al. Transbronchial needle injection: a systematic review of a new diagnostic and therapeutic paradigm. Respiration. 2006; 73:78-89.
18. Ward R F et al. Mitomycin-C trtmnt of tracheal cicatrix aftr trachl recnstructn. Int J. Ped. Otolrhinolaryngol. 1998; 44:221-6.
19. Ksimoff, M. Management of Obstructing Airway Lesions in Patients with Lung Cancer.
20. Simoff, M. J. Endobronchial Management of Advanced Lung Cancer. Cancer Control. 2001; 8(4):337-343.
21. Asimakopoulos, G., et al. Cryosurgery—Malignant Endbrnchial Tumors: Analyis of outcome. Chest. 2005; 127:2007-2014.
22. Ernst, A. et al. Central Airway Obstruction. Am J Respirat Critical Care Med. 2004; 169:1278-1297.
23. Wood, D E, et al. Airway stenting for malignant and benign tracheobronchial stenosis. Ann Thorac Surg. 2003; 76:167.
24. K. Moghissi. Role of brnchscopic photodynamic therapy in LC management. Curr Opin Pulm Med. 2004; 10(4):256-60.
25. Celikoglu S. I., et al. Bronchoscopic intratumoral chemotherapy of lung cancer. Lung Cancer. 2008; 61:1-12.
26. Gagnadoux F, et al. Aerosolized chemotherapy. J Aerosol Med Pulm Drug Deliv. 2008; 21(1):61-70.
27. C. W. Seymour, et al. Transbronchial Needle Injection: A Systematic Review of a New Diagnostic and Therapeutic Paradigm. Respiration. 2006; 73:78-89.
28. MCC Medical Center Cologne. “Loco-regional chemotherapy and chemothrombolisation.” Oncology; 2009.
29. USFDA. “PRECISE Trial. Study of Gliadel Wafer in patients with recurrent Glioblastoma.” www.fda.gov
30. Qing-Shan Pei and Ji-Yong Liu. Radiology 2008; 247:574-581
31. Tamada, J. & R. Langer. The dev. of polyanhydrides for drug delivery apps. J. Biomater. Sci. Polym. Ed. 1992; 3(4):315-53.
32. Narasimhan B et al. Undrstanding phase behavior of polyanhydride blends using scattering, microscopy, and molecular simulations. Polymer. 2004; 45(1)0:3329-3340.
33. McCann, D. L., et al. Charzn. chem. struct. & morphlg of eroding polyanhydrides-1H NMR. Polymer. 1999; 40:2151-62.
34. ICH Expert Working Group. ICH Guidelines Validation of Analytical Procedures: Q2 (R1). http://www.ich.org.
35. www.fda.gov. Guidance for Industry Bioanalytical Method Validation. FDA, CDER, CVM. 2001. BP.
36. Vearick, S B, et al. Dev. & testing of Nitinol tracheal stent. J Biomed Mat Res B Appl Biomat. 2007; 83(1):216-21.
37. S. Leroy, et al. A New, Self Expanding, Removable, Covered Metal Tracheobronchial Stent: Results of an Animal Trial. J. Bronchol. 2006; 13(3):111-113.
38. Xavier R G, et al. Development of a modified. Dumon stent for tracheal applications: an experimental study in dogs. J. Bras. Pneumol. 2008; 34(1):21-6.
39. Marquette C H, et al. Experimental model of tracheobronchial stenoses: a useful tool for evaluating airway stents. Ann Thorac Surg. 1995; 60:651-656.
40. Verkindre C, et al. Morphological changes induced by extnsve endbrnchial elctrcautry. Eur Resp J. 1999; 14:796-9.
41. DiMassi et al. The price of innovation.—new estimates of drug development costs. J Health Econ. 2003; 22:151-85.
42. Eleftherios P. Mamounas. Tailoring Loco-Regional Therapy with Neoadjuvant Chemotherapy: Another Step in the Right Direction. Annals of Surgical Oncology. 2004; 11.10:888-891.
43. 49. R. S. Kerbel, G. Klement, K. I. Pritchard and B. Kamen. Continuous low-dose antiangiogenic/metronomic chemotherapy: from the research laboratory into the oncology clinic. Annals of Oncology. 2002; 13:12-15, 2002.
44. Kerbel R S, Kamen B A. The anti-angiogenic basis of metronomic chemotherapy. Nat Rev Cancer. 2004; 4:423-436.
45. Elstad N L, and Fowers K D (2009) OncoGel (ReGel/paclitaxel)—Clinical applications for a novel paclitaxel delivery system. Adv Drug Delivery Rev. 2009 May 5.
46. Mayoral W, Fleischer D, Salcedo J, et al. (2000) Nonmalignant obstruction is a common problem with metal stents in the treatment of esophageal cancer. Gastrointest Endosc. 51:556-9.
47. Melchert M and List A. (2007) The thalidomide saga. Int. J. Biochem & Cell Biol 39:1489-1499.
48. Franks M E, Macpherson G R, Figg W D. (2004) Thalidomide. Lancet 363:1802-11 Eleutherakis-Papaiakovou V, Bamias A,
49. Desmettre et al. Major Review. Survey of Ophthamology, vol 45, no. 1, 2000. Fluorescence Properties and Metabolic Features of Indocyanine Green (ICG) as Related to Angiography. T.
50. Saxena et al. Journal of Photochemistry and Photobiology B: Biology Volume 74, Issue 1, 19 Mar. 2004, Pages 29-38. Enhanced photo-stability, thermal-stability and aqueous-stability of indocyanine green in polymeric nanoparticulate systems
51. Romila M. et al. Preparation and characterization of a polymeric (PLGA) nanoparticulate drug delivery system with simultaneous incorporation of chemotherapeutic and thermo-optical agents. Colloids and Surfaces B: Biointeifaces, In Press, Corrected Proof, Available online 4 Sep. 2009
52. Sophie Boddington et al., Contrast Media & Molecular Imaging. Volume 3 Issue 5, Pages 191-197 Improved fluorescence of indocyanine green in vitro and in vivo after simple cooling Procedures.
53. Cesar et al. The use of indocyanine green in the measurement of hepatic blood flow and as a test of hepatic function. J. Clin. Sci. 1961; 21: 43-57.

I claim:

1. A drug delivery system, the drug delivery system comprising a support, a first polymer matrix, a fluorescent dye, and at least one drug.

2. The drug delivery system of claim 1, wherein the first polymer matrix comprises a first material that is substantially susceptible to degradation by a composition having biological enzyme activity.

3. The drug delivery system of claim 1, wherein the first polymer matrix comprises a second material that is substantially resistant to degradation by a composition having biological enzyme activity.

4. The drug delivery system of claim 1, wherein the first polymer matrix comprises one first material and one second material, wherein the first material is substantially susceptible to degradation by a composition having biological enzyme activity and the second material is substantially resistant to degradation by a composition having biological enzyme activity.

5. The drug delivery system of claim 1, wherein the support is selected from the group consisting of a stent, a balloon, and a second polymer matrix.

6. The drug delivery system of claim 5 wherein the stent is selected from the group consisting of a tubular structure, a metallic self-expanding stent, a balloon expandable metallic stent, a self-expanding stent, and a stent-graft.

7. The drug delivery system of claim 5 wherein the second polymer matrix comprises a first material that is substantially susceptible to degradation by a composition having biological enzyme activity.

8. The drug delivery system of claim 5 wherein the second polymer matrix comprises a second material that is substantially resistant to degradation by a composition having biological enzyme activity.

9. The drug delivery system of claim 5 wherein the second polymer matrix comprises one first material and one second material, wherein the first material is substantially susceptible to degradation by a composition having biological enzyme activity and the second material is substantially resistant to degradation by a composition having biological enzyme activity.

10. The drug delivery system of claim 1 wherein the first polymer matrix comprises a composition selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof.

11. The drug delivery system of claim 1 wherein the first polymer matrix comprises a copolymer together with monomers of a hydrophilic polymer selected from the group consisting of polyvinylpyrrolidone, polyvinylalcohol, polyhydroxyethylmethacrylate, polyacrylamide, polymethacrylamide, and polyethyleneglycol.

12. The drug delivery system of claim 5 wherein the second polymer matrix comprises a composition selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof.

13. The drug delivery system of claim 5 wherein the second polymer matrix comprises a copolymer together with monomers of a hydrophilic polymer selected from the group consisting of polyvinylpyrrolidone, polyvinylalcohol, polyhydroxyethylmethacrylate, polyacrylamide, polymethacrylamide, and polyethyleneglycol.

14. The drug delivery system of claim 1 wherein the fluorescent dye is indocyanine green

15. The drug delivery system of claim 1 further comprising a pharmaceutical formulation.

16. The drug delivery system of claim 15 wherein the pharmaceutical formulation comprises the drug and a suitable pharmaceutical carrier.

17. The drug delivery system of claim 1 wherein the drug is selected from the group consisting of thalidomide, docetaxel, etoposide, irinotecan, paclitaxel, teniposide, topotecan, vinblastine, vincristine, vindesine, busulfan, improsulfan, piposulfan, aziridines, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, temozolomide, aclacinomycinsa actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin, denopterin, edatrexate, methotrexate, piritrexim, pteropterin, TOMUDEX, trimetrexate, cladribine, fludarabine, 6-mercaptopurine, thiamiprine, thioguanine, ancitabine, azacitidine, 6-azauridine, carmofur, cytarabine, doxifluridine, emitefur, enocitabune, floxuridine, fluorouracil, gemcitabine, tegafur, L-asparaginase, interferon-α, interferon-β, interferon-γ, interleukin-2, lentinan, propagermanium, PSK, roquinimex, sizofican, ubenimex, carboplatin, cisplatin, miboplatin, oxaliplatin, aceglarone, amsacrine, bisantrene, defosfamide, demecolcine, diaziquone, eflomithine, elliptinium acetate, etoglucid, fenretinide, gallium nitrate, hydroxyurea, lonidamine, miltefosine, mitoguazone, mitoxantrone, mopidamol, nitracine, pentostain, phenamet, podophyllinic acid 2-ethyl-hydrazide, procabazine, razoxane, sobuzoxane, spirogermanium, tenuzonic acid, triaziquone, 2,2′,2″trichlorotriethylamine, urethan, calusterone, dromostanolone, epitiostanol, mepitiostane, testolacone, aminoglutethimide, mitotane, trilostane, bicalutamide, flutamide, nilutamide, droloxifene, tamoxifen, toremifene, aminoglutethimide, anastrozole, fadrozole, formestane, letrozole, fosfestrol, hexestrol, polyestradiol phosphate, buserelin, goserelin, leuprolide, triptorelin, chlormadinone acetate, medroxyprogesterone, megestrol acetate, melengestrol, porfimer sodium, batimastar, folinic acid, salicylates, salsalate, mesalamine, diflunisal, choline magnesium trisalicylate, diclofenac, diflunisal, etodolac, fenoprofen, flurbiprofen, ibuprofen, indomethacin, mefenamic acid, nabumetone, naproxen, piroxicam, phenylbutazone, ketoprofen, S-ketoprofen, ketorolac tromethamine, sulindac, tolmetin, beclomethasone, betamethasone, cortisone, dexamethasone, fluocinolone, flunisolide, fluticasone proprionate, fluorinated-corticoids, triamcinolone-diacetate, hydrorcortisone, clobetasol, prednisolone, methylprednisolone, prednisone, finasteride, adenocorticosteroids, cyclosporin, rapamycin, everolimus, sutinib maleate, gefitinib, and erlotinib.

18. The drug delivery system of claim 17 wherein the drug is selected from the group consisting of paclitaxel, thalidomide, neomycin, 5-fluorouracil, irinotecan, sutinib maleate, gefitinib, erlotinib, and clobetasol.

19. The drug delivery system of claim 5 wherein the stent comprises a metal selected from the group consisting of nickel-titanium alloy, chromel, stainless steel, copper, gold, platinum, silver, and titanium.

20. The drug delivery system of claim 5 wherein the stent comprises a material selected from the group consisting of conductive epoxy, conductive polymers, barium sulfate, titanium oxide, silicone, polyurethane, polyethylene, acrylonitrile butadiene styrene, polycarbonate, polypropylene, styrene, polyamide, polyimide, PEEK, PEBAX, polyester, PVC, fluoropolymers, and co-polymers thereof.

21. A method for treating cancer in a subject, the method comprising the steps of: (i) providing a first polymer matrix, the first polymer matrix comprising at least one pair of drugs in combination and a fluorescent dye, wherein the first polymer matrix is in a phase suitable for placing in a body passageway and wherein the first polymer matrix comprises a compound that allows the drug to be delivered from the first polymer matrix; (ii) introducing the first polymer matrix into the body passageway proximal to the cancer in the subject; (iii) allowing the drug to be delivered from the first polymer matrix to a vicinity adjacent to the cancer, the drug thereby effecting biological activity upon the cancer, the method resulting in treating the cancer.

22. The method of claim 21 wherein the body passageway is selected from the group consisting of coronary artery, carotid artery, aorta, pulmonary artery, vein, capillary, trachea, bronchus, bronchioles, oesaphagus, bile duct, fallopian tubes, urethra, colon, bladder, pancreatic passageway, nasal passageways, male reproductive tract, female reproductive tract, small intestine, large intestine, cranial sinus, and brain sinus.

23. The method of claim 21 wherein the first polymer matrix comprises a polymer selected from the group consisting of bio-degradable polymers, non-bio-degradable polymers, and combinations thereof.

24. The method of claim 21 wherein the phase of the first polymer matrix is selected from the group consisting of a liquid, a gel, a solid, and combinations thereof.

25. The method of claim 21 wherein the first polymer matrix comprises a polymer selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof.

26. The method of claim 21 wherein the drug is selected from the group consisting of thalidomide, docetaxel, etoposide, irinotecan, paclitaxel, teniposide, topotecan, vinblastine, vincristine, vindesine, busulfan, improsulfan, piposulfan, aziridines, benzodepa, carboquone, meturedepa, uredepa, altretamine, triethylenemelamine, triethylenephosphoramide, triethylenethiophosphoramide, chlorambucil, chloraphazine, cyclophosphamide, estramustine, ifosfamide, mechlorethamine, mechlorethamine oxide hydrochloride, melphalan, novembichin, perfosfamide, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, temozolomide, aclacinomycinsa actinomycin anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin, denopterin, edatrexate, methotrexate, piritrexim, pteropterin, TOMUDEX, trimetrexate, cladribine, fludarabine, 6-mercaptopurine, thiamiprine, thioguanine, ancitabine, azacitidine, 6-azauridine, carmofur, cytarabine, doxifluridine, emitefur, enocitabune, floxuridine, fluorouracil, gemcitabine, tegafur, L-asparaginase, interferon-α, interferon-β, interferon-γ, interleukin-2, lentinan, propagermanium, PSK, roquinimex, sizofican, ubenimex, carboplatin, cisplatin, miboplatin, oxaliplatin, aceglarone, amsacrine, bisantrene, defosfamide, demecolcine, diaziquone, eflomithine, elliptinium acetate, etoglucid, fenretinide, gallium nitrate, hydroxyurea, lonidamine, miltefosine, mitoguazone, mitoxantrone, mopidamol, nitracine, pentostain, phenamet, podophyllinic acid 2-ethyl-hydrazide, procabazine, razoxane, sobuzoxane, spirogermanium, tenuzonic acid, triaziquone, 2,2′,2″trichlorotriethylamine, urethan, calusterone, dromostanolone, epitiostanol, mepitiostane, testolacone, aminoglutethimide, mitotane, trilostane, bicalutamide, flutamide, nilutamide, droloxifene, tamoxifen, toremifene, aminoglutethimide, anastrozole, fadrozole, formestane, letrozole, fosfestrol, hexestrol, polyestradiol phosphate, buserelin, goserelin, leuprolide, triptorelin, chlormadinone acetate, medroxyprogesterone, megestrol acetate, melengestrol, porfimer sodium, batimastar, folinic acid, salicylates, salsalate, mesalamine, diflunisal, choline magnesium trisalicylate, diclofenac, diflunisal, etodolac, fenoprofen, flurbiprofen, ibuprofen, indomethacin, mefenamic acid, nabumetone, naproxen, piroxicam, phenylbutazone, ketoprofen, S-ketoprofen, ketorolac tromethamine, sulindac, tolmetin, beclomethasone, betamethasone, cortisone, dexamethasone, fluocinolone, flunisolide, fluticasone proprionate, fluorinated-corticoids, triamcinolone-diacetate, hydrorcortisone, clobetasol, prednisolone, methylprednisolone, prednisone, finasteride, adenocorticosteroids, cyclosporin, rapamycin, everolimus, sutinib maleate, gefitinib, and erlotinib.

27. The method of claim 26 wherein the drug is selected from the group consisting of paclitaxel, thalidomide, neomycin, and clobetasol.

28. The method of claim 21 wherein the first polymer matrix is introduced into the body passageway in combination with a support.

29. The method of claim 28 wherein the support is selected from the group consisting of a stent, a balloon, and a second polymer matrix.

30. The method of claim 29 wherein the stent is selected from the group consisting of a tubular structure, a metallic self-expanding stent, a balloon expandable metallic stent, a self-expanding stent, and a stent-graft.

31. The method of claim 29 wherein the stent comprises a metal selected from the group consisting of nickel-titanium alloy, chromel, stainless steel, copper, gold, platinum, silver, and titanium.

32. The method of claim 29 wherein the second polymer matrix comprises a polymer selected from the group consisting of partially esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, and polyprotains, and completed esterified polymers of acrylic acid, methacrylic acid, polyphosphazenes, polycarbonates, polylactic acid, polyglycolic acid, lactic acid, glycolic acid, polyhydroxybutyric acid, polyorthoesters, polyanhydrides, polysiloxanes, polycaprolactone, polysaccharides, polyprotains, and copolymers thereof.

33. The method of claim 21 wherein the cancer is selected from the group consisting of a tumor, vascular smooth muscle, endothelium, extracellular matrix, platelet aggregate, a thrombus, fibrin matrix, epidermal tissue, and neurological tissue.

34. The method of claim 21 wherein the cancer is selected from the group consisting of oral-pharyngeal carcinoma (adenocarcinoma), esophageal carcinoma (squamous cell, adenocarcinoma, lymphoma, melanoma), gastric carcinoma (adenocarcinoma, linitis plastica, lymphoma, leiomyosarcoma), small bowel tumors (adenomas, leiomyomas, lipomas, adenocarcinomas, lymphomas, carcinoid tumors), colon cancer (adenocarcinoma) and anorectal cancer), pancreatic carcinoma (ductal adenocarcinoma, islet cell tumors, cystadenocarcinoma), cholangiocarcinoma and hepatocellular carcinoma), and carcinoma of the lung and/or tracheal/bronchial passageways (small cell lung cancer, non-small cell lung cancer).

35. A drug delivery system for use in the treatment of or prevention of an obstruction in a body passageway, comprising the drug delivery system of claim 1.

36. The drug delivery system of claim 1, further comprising biological activity, wherein the biological activity is anti-tumor activity.

37. The drug delivery system of claim 36, wherein the anti-tumor activity is directed to a neoplastic disease, wherein the neoplastic disease is a gastrointestinal disease.

38. The drug delivery system of claim 37, wherein the gastrointestinal disease is selected from the group consisting of oral-pharyngeal carcinoma (adenocarcinoma), esophageal carcinoma (squamous cell, adenocarcinoma, lymphoma, melanoma), gastric carcinoma (adenocarcinoma, linitis plastica, lymphoma, leiomyosarcoma), small bowel tumors (adenomas, leiomyomas, lipomas, adenocarcinomas, lymphomas, carcinoid tumors), colon cancer (adenocarcinoma) and anorectal cancer), pancreatic carcinoma (ductal adenocarcinoma, islet cell tumors, cystadenocarcinoma), cholangiocarcinoma and hepatocellular carcinoma), and carcinoma of the lung and/or tracheal/bronchial passageways (small cell lung cancer, non-small cell lung cancer).

39. The drug delivery system of claim 1 wherein the support comprises an optional marker, the marker selected from the group consisting of a radiopaque composition, a radiopaque dye, a radio-opaque material, a magnet, an echogenic material, an ion source, and a radio-isotope.

40. The method of claim 21, wherein the cancer is selected from the group consisting of oral-pharyngeal carcinoma (adenocarcinoma), esophageal carcinoma (squamous cell, adenocarcinoma, lymphoma, melanoma), gastric carcinoma (adenocarcinoma, linitis plastica, lymphoma, leiomyosarcoma), small bowel tumors (adenomas, leiomyomas, lipomas, adenocarcinomas, lymphomas, carcinoid tumors), colon cancer (adenocarcinoma) and anorectal cancer), pancreatic carcinoma (ductal adenocarcinoma, islet cell tumors, cystadenocarcinoma), cholangiocarcinoma and hepatocellular carcinoma), and carcinoma of the lung and/or tracheal/bronchial passageways (small cell lung cancer, non-small cell lung cancer).

41. The drug delivery system of claim 1 further comprising at least one pair of drugs in combination, wherein the first polymer matrix comprises a first material that is substantially susceptible to degradation by a composition having biological enzyme activity, wherein a first drug of the pair comprises cytotoxic activity, wherein a second drug of the pair comprises non-cytotoxic activity, wherein the second drug comprises biological activity selected from the group consisting of targeting compound activity, immunomodulatory activity, anti-angiogenic activity, and anti-inflammatory activity, and wherein the pair of drugs in combination are drugs with complimentary mechanism of actions.

42. The drug delivery system of claim 41, wherein the pair of drugs in combination are thalidominde and paclitaxel, wherein the first polymer matrix comprises a polyanhydride, wherein the fluorescent dye is indocyanine green, and wherein the support comprises NITINOL.

43. The drug delivery system of claim 41, wherein the pair of drugs in combination are 5-fluorouracil and paclitaxel, wherein the first polymer matrix comprises a polyanhydride, wherein the fluorescent dye is indocyanine green, and wherein the support comprises NITINOL.

44. The method of claim 21, wherein the pair of drugs in combination are thalidominde and paclitaxel, wherein the first polymer matrix comprises a polyanhydride, wherein the fluorescent dye is indocyanine green, and wherein the support comprises NITINOL.

45. The method of claim 21, wherein the pair of drugs in combination are 5-fluorouracil and paclitaxel, wherein the first polymer matrix comprises a polyanhydride, wherein the fluorescent dye is indocyanine green, and wherein the support comprises NITINOL.

46. The drug delivery system of claim 35, wherein the pair of drugs in combination are thalidominde and paclitaxel, wherein the first polymer matrix comprises a polyanhydride, wherein the fluorescent dye is indocyanine green, and wherein the support comprises NITINOL.

47. The drug delivery system of claim 35, wherein the pair of drugs in combination are 5-fluorouracil and paclitaxel, wherein the first polymer matrix comprises a polyanhydride, wherein the fluorescent dye is indocyanine green, and wherein the support comprises NITINOL.
